(19) United States
(12) Patent Application Publication
(10) Pub. No.: US 2022/0280450 A1
(43) Pub. Date: Sep. 8, 2022

(54) PREVENTION AND TREATMENT OF CORONAVIRUS AND RELATED RESPIRATORY INFECTIONS

(71) Applicant: PhilERA New Zealand Ltd., Auckland (NZ)

(72) Inventors: Joseph FORTUNAK, Silver Spring, MD (US); Garth COOPER, Auckland (NZ); Margaret COOPER, Auckland (NZ)

(21) Appl. No.: 17/687,444

(22) Filed: Mar. 4, 2022

Related U.S. Application Data
(60) Provisional application No. 63/157,545, filed on Mar. 5, 2021.

(51) Int. Cl. A61K 31/132; A61K 33/24; A61K 31/198; A61K 45/06; A61K 31/4706; A61K 31/635; A61K 31/519; A61K 31/513; A61K 31/427; A61K 31/675; A61K 31/53; A61K 38/21

(52) U.S. Cl. CPC A61K 31/132; A61K 33/24; A61K 31/198; A61K 45/06; A61K 31/4706; A61K 31/635; A61K 31/519; A61K 31/513; A61K 31/427; A61K 31/675; A61K 31/53; A61K 38/215

(57) ABSTRACT

The present disclosure relates to compositions comprising copper-depriving compounds, including copper chelators, useful for the prophylaxis and treatment of SARS-CoV-2, SARS-CoV-2 variants and mutations, and other coronavirus infections.

### FIELD

The inventions relate generally to coronaviruses and coronavirus infections, and to compounds that deprive coronaviruses of copper, including copper chelators and copper transporter antagonists.

### INCORPORATION BY REFERENCE

All U.S. patents, U.S. patent application publications, foreign patents, foreign and PCT published applications, articles and other documents, references and publications noted herein, and all those listed as References Cited in any patent or patents that issue herefrom, are hereby incorporated by reference in their entirety. The information incorporated is as much a part of this application as if all the text and other content is repeated in the application and will be treated as part of the text and content of this application as filed.

### BACKGROUND

The following includes information that may be useful in understanding the present inventions. It is not an admission that any of the information is prior art, or relevant, to the presently described or claimed inventions, or that any publication or document that is specifically or implicitly referenced is prior art or a reference that may be used in evaluating patentability of the described or claimed inventions.

Though much of the world is hearing about them for the first time, coronaviruses are a large family of related viruses responsible for respiratory infections in vertebrates such as livestock, birds, bats and rodents. Coronaviruses are typically zoonotic, meaning they can be transmitted between species-often when individuals are in close proximity-allowing transmission via droplets produced through coughing or sneezing. In humans, illnesses can range from common cold-like symptoms to more severe diseases such as the Middle East Respiratory Syndrome (MERS-CoV), first reported in 2012 and Severe Acute Respiratory Syndrome (SARS-CoV) in 2003. A novel coronavirus, now known as SARS-CoV-2 has emerged as a major global threat to human health in 2020 and is responsible for the infectious disease COVID-19.

Coronaviruses are a group of related viruses that cause diseases in humans and animals, most of which circulate among such animals as pigs, camels, bats, deer, minks and cats. Sometimes those viruses jump to humans—called a spillover event—and can cause disease. In humans, coronaviruses cause respiratory tract infections that are typically mild. Four of the seven known coronaviruses that sicken people cause only mild to moderate disease. Three can cause more serious, even fatal, disease. SARS coronavirus (SARS-CoV) emerged in November 2002 and caused severe acute respiratory syndrome (SARS). That virus disappeared by 2004. Middle East respiratory syndrome (MERS) was caused by the MERS coronavirus (MERS-CoV). Transmitted from an animal reservoir in camels, MERS is identified in September 2012 and continues to cause sporadic and localized outbreaks.

The third novel coronavirus to emerge in this century is called SARS-CoV-2. SARS-CoV-2 is a positive-sense and single-stranded RNA virus of zoonotic origin belonging to Betacoronavirus lineage B. It causes coronavirus disease 2019 (COVID-19), which is said to have emerged from China in December 2019 and was declared a global pandemic by the World Health Organization on Mar. 11, 2020. Coronavirus disease 2019 infections caused by the severe acute respiratory syndrome coronavirus 2 (SARS-CoV-2) have spread globally since late 2019, resulting in the 2019-20 coronavirus pandemic. Preliminary research has yielded case fatality rate numbers between 1% and 3% for COVID-19 and the outbreak in 2019-2020 has reportedly, according to the Centers for Disease Control and Prevention (CDC), led to over 6.7 million confirmed infections in the US alone and over 199,000 deaths as of mid-September 2020, with deaths being mostly amongst the elderly and those with comorbidities.

It is believed that, in those with underlying health conditions or comorbidities, COVID-19 has an increasingly rapid and severe progression, leading to death in some cases. From what is known at the moment, patients with COVID-19 disease who have comorbidities, such as hypertension or diabetes mellitus, are more likely to develop a more severe course and progression of the disease. Furthermore, older patients, especially those 65 years old and above who have comorbidities and are infected, have an increased admission rate into the intensive care unit (ICU) and a higher risk of mortality from the COVID-19 disease.

In a recent meta-analysis study on COVID-19 comorbidities with a total of 1786 patients, the most common reported comorbidities are hypertension (15.8%), cardiovascular and cerebrovascular conditions (11.7%), and diabetes (9.4%). Less common comorbidities are coexisting infection with HIV and hepatitis B (1.5%), malignancy (1.5%), respiratory illnesses (1.4%), renal disorders (0.8%), and immunodeficiencies (0.01%). Sanyuaolu, et al., Comorbidity and its Impact on Patients with COVID-19 SN Compr Clin Med. 2020 Jun. 25:1-8.

In the past, therapeutic strategies for microbial pathogens primarily targeted pathogen genes and proteins. This strategy works well for anti-bacterial drugs in most of the cases. It has much less success in anti-virus therapy as viral genes have the intrinsic ability to mutate frequently and can become resistant to vaccines and drugs due to less error correction activity of their nucleotide polymerases in virus replication, as well as multiple sub-families with small differences in target genes. In other words, viruses can either mutate or become resistant to antiviral drugs. For example, although flu vaccines have been widely distributed, the CDC estimates the efficacy of flu vaccines against both influenza A and B viruses at only 40-60%.

In the US, three neuraminidase inhibitors (NAI) are recommended by the CDC: oseltamivir, zanamivir, and peramivir. Most of the recently circulating influenza viruses have been susceptible to the NAI antiviral medications, but recent virus isolates from patients show significant drug resistance, even in the same year of drug launch. There is another class of influenza antiviral drug (amantadine and rimantadine) that are not recommended for use in the US because about half of flu A viruses are resistant to these drugs and they are not effective against the influenza B virus. Also, these antiviral agents must generally be used within 48 hours of the onset of influenza symptoms to be effective. But most of the time, when severe symptoms occur, it already past the time and is at a late or very late stage. As a consequence of the current anti-virus strategies, although broad vaccines and drugs targeting virus proteins have been developed in the last decade, the influenza virus, for example, is still a highly life-threatening disease, as is the SARS-CoV-2 virus and other coronaviruses. The severity of the SARS-CoV-2 virus has created a high burden to the health of people throughout the world. However, the current pipeline of drug discovery still focuses mainly on viral proteins.

Some research states that copper itself can effectively help to prevent the spread of respiratory viruses, which are linked to SARS and MERS. Researchers reported that human coronavirus 229E, for example, can remain infectious on common surface materials for several days, but is rapidly destroyed on copper. S. L. Warnes, et al. Human coronavirus 229E remains infectious on common touch surface materials. mBio, November 2015 DOI: 10.1128/mBio.01697-15. Over the past few months, there has been a surge in the market for materials laced with copper—including face masks, bedsheets, and socks—with manufacturers touting the metal's germ-killing ability. Experts caution, however, that copper is not a proven remedy, including against the new coronavirus, SARS-CoV-2.

A number of copper-depriving compounds, including copper chelators, are generally recognized as safe and effective, and have been used therapeutically in, for example, the treatment of Wilson's Disease. Certain copper chelators have also been described for use in treating certain disorders, including cardiovascular, glucose and vascular disorders. Prior teachings relating to copper chelators are described in, for example, U.S. Pat. No. 10,543,178 (use of a succinic acid addition salt of triethylenetetramine to treat diabetic neuropathy), U.S. Pat. No. 9,993,443 (use of a succinic acid addition salt of triethylenetetramine to treat tissue damage associated with specific cardiac, glucose related and vascular disorders), U.S. Pat. No. 8,987,244 (use of various chelators, including trientine, 2,2,2 tetramine tetrahydrochloride and 2,3,2 tetramine tetrahydrochloride to lower copper (II) values in patients with tissue damage in myocardial tissue, kidney tissue, eye tissue, nerve tissue, and vascular tissue), U.S. Pat. No. 8,563,538 (use of 2,3,2 tetramine compositions in methods of treating heart failure in a non-diabetic human subject, including 2,3,2 tetramine hydrochloride salts, e.g., 2,3,2 tetramine tetrahydrochloride), U.S. Pat. No. 8,034,799 (methods of treating heart failure in a non-diabetic human subject with an agent capable of reducing copper levels, for example, copper (II), including copper chelators such as trientine, as well as 2,3,2 tetramine, D-penicillamine, N-acetylpenicillamine, trithimolybdate, and tetrathimolybdate), and U.S. Pat. No. 7,928,094 (use of triethylenetetramine dihydrochloride to treat one or more conditions associated with long-term complications of diabetes).

As of yet there are no vaccines or new antiviral drugs to prevent or treat human coronavirus infections. There exists a need to develop compositions useful in preventing and/or minimizing the risk of coronavirus infections, particularly SARS-CoV-2, which leads to COVID-19 disease. The present disclosure satisfies these needs and provides methods and compositions to deprive coronaviruses of copper.

### BRIEF SUMMARY

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this introduction, which is included for purposes of illustration only and not restriction.

In one aspect, a method of preventing or reducing the risk of infection or treating an infection in a subject caused by exposure to a coronavirus is provided, e.g., SARS-CoV-2, the method comprising administering to a subject, either before or after the exposure, a composition comprising or consisting essentially of or consisting of a compound that deprives a coronavirus of copper, including, e.g., a copper(I) and/or a copper(II) chelator, a copper binding agent, an agent that lowers total copper values in a subject, or an agent that lowers intracellular copper, for example, by knockdown or inhibition of host cell copper transporters, wherein the composition is formulated for oral, nasal or parenteral administration, including by administration to the nasal vestibule or passages of the subject, wherein the method results in reducing infectious coronavirus organisms and/or virus particles in the subject, preventing coronavirus infection or reducing the risk of coronavirus infection in the subject, or reducing or eliminating an existing coronavirus infection.

In another aspect, the invention comprises a method of preventing or reducing the risk of infection or treating an infection in a subject caused by exposure to a copper-requiring coronavirus, the method comprising or consisting essentially of or consisting of administering to the subject, either before or after the exposure, a composition comprising an agent effective to deprive a coronavirus of copper in an amount effective to reduce or cause defects in viral growth and replication, including agents that lower copper(1) content, copper(II) content, copper values content, or intracellular copper content in the subject, wherein the method results in reducing infectious coronavirus organisms and/or coronavirus virus particles and/or preventing coronavirus infection or reducing the risk of coronavirus infection in the subject, or reducing or eliminating an existing coronavirus infection.

In other embodiments of methods of the invention, administration of the copper-depriving compound is endotracheal, endosinusial, intrabronchial, intracavernous, intrasinal, intrapulmonary or transmucosal.

In one embodiment, the agent effective to lower the copper values content in a subject and deprive a copper-requiring coronavirus of copper is a copper chelating compound. In another embodiment, the agent effective to lower the copper values content in the subject comprises or consists essentially of or consists of an agent that binds or chelates copper(I). In another embodiment, the agent effective to lower the copper values content in the subject comprises or consists essentially of or consists of an agent that binds or chelates copper(II). In another embodiment, the agent effective to lower the copper values content in the subject comprises or consists essentially of or consists of an agent that binds or chelates both copper(I) and copper(II).

In one embodiment, the agent effective to lower the copper values content in a subject or otherwise to deprive a coronavirus of copper comprises or consists essentially of or consists of an agent that may be selected from the group consisting of D-penicillamine; N-acetylpenicillamine; triethylenetetramine (also called TETA, TECZA, trien, triene and trientine), and pharmaceutically acceptable salts thereof; trithiomolybdate, tetrathiomolybdate, ammonium tetrathiomolybdate, choline tetrathiomolybdate; bis-choline tetrathiomolybdate (thiomolybdate USAN, trade name Decuprate), 2,2,2 tetramine tetrahydrochloride; 2,3,2 tetramine tetrahydrochloride; ethylenediaminetetraacetic acid salts (EDTA, a non-preferred non-specific metal binder, administered with care to avoid toxicity); diethylenetriaminetetraacetic acid (DPTA, a non-preferred non-specific metal binder, administered with care to avoid toxicity that is due to chelation of essential metals, such as Zn and Mn); 5,7,7′12,14,14′hexaxmethyl-1,4,8,11 tetraazacyclotretradecane; 1,4,8,11 tetraazacyclotretradecane, including cyclam S, cylams, and copper-chelating cyclam derivatives, e.g., Bn-cyclam-EtOH, oxo-cyclam-EtOH and oxo-Bn-cyclam-EtOH, (HOCH₂CH₂CH₂)₂(PhCH₂)₂Cyclam and (HOCH₂CH₂CH₂)₂(4-CF₃ PhCH₂)₂Cyclam; 1,4,8,11-tetraazacyclotetradecane-1,4,8,11-tetraacetic acid; 1,4,8,11-tetraazabicyclo[6.6.2]hexadecane; 4,11-bis(N,N-diethyl-amidomethyl)-1,4,8,11-tetraazabicyclo[6.6.2]hexadecane; 4,11-bis(amidoethyl)-1,4,8,11-tetraazabicyclo[6.6.2]hexadecane; melatonin; cyclic 3-hydroxymelatonin (30HM); N(1)-acetyl-N(2)-formyl-5-methoxykynuramine (AFMK); N(1)-acetyl-5-methoxykynuramine (AMK); N,N′-diethyldithiocarbamate; bathocuproinedisulfonic acid; bathocuprinedisulfonate; trimetazidine; triethylene tetramine tetrahydrochloride; 2,3,2-tetraamine; 1,10-orthophenanthroline; 3,4-dihydroxybenzoic acid; 2,2′-bicinchinonic acid; diamsar; 3,4′,5, trihydroxystilbene (resveratrol); mercaptodextran; disulfiram (Antabuse); sarcophagine; DiAmSar; diethylene triamine pentaacetic acid; and calcium trisodium diethylenetriaminepentaacetate; neocuproine; bathocuproine; and carnosine.

In one embodiment, the agent to deprive a coronavirus of copper reduces total copper in the subject.

In another embodiment, the agent to deprive a coronavirus of copper reduces intracellular copper in the subject, particularly in coronavirus host cells in the subject.

In another embodiment, the agent to deprive a coronavirus of copper maintains total copper in the subject within the normal human serum or plasma range of about 0.8-1.2 milligrams/L, or about 10-25 micromoles/L. In another embodiment, the agent to deprive a coronavirus of copper maintains total copper in the subject within at least about 70% of the normal range of about 0.8-1.2 milligrams/L or about 10-25 micromoles/L, e.g., at least about 75%. In another embodiment, the agent to deprive a coronavirus of copper maintains total copper in the subject within about 75% to about 85%, or about 85% to about 95% the normal range of copper in human plasma or serum. In one aspect of the methods of the invention, the copper status of a subject provided an agent to deprive a coronavirus of copper is determined by evaluating copper in the urine of the subject.

In another embodiment, the agent preferentially binds Cu¹⁺. In another embodiment, the agent preferentially binds Cu²⁺. In one embodiment, the agent that preferentially binds Cu²⁺ is triethylenetetramine disuccinate. In another embodiment, the agent binds both Cu¹⁺ and Cu²⁺. In one embodiment, the agent that preferentially binds both Cu¹⁺ and Cu²⁺ is a penicillamine copper chelator, preferably D-penicillamine.

In one embodiment, the copper-depriving compound is a triethylenetetramine.

In another embodiment, the triethylenetetramine is a hydrochloride salt of triethylenetetramine. In one embodiment, the triethylenetetramine hydrochloride salt is triethylenetetramine dihydrochloride. In another embodiment, the triethylenetetramine hydrochloride salt is triethylenetetramine tetrahydrochloride. In another embodiment, the triethylenetetramine is a succinate salt of triethylenetetramine. In one embodiment, the triethylenetetramine succinate salt is triethylenetetramine disuccinate.

In one aspect of the invention, the method employs a pharmaceutical composition comprising substantially pure triethylenetetramine disuccinate. In another aspect the method employs a pharmaceutical composition comprising substantially pure triethylenetetramine disuccinate and a pharmaceutically acceptable excipient. In another aspect, the method employs a pharmaceutical composition comprising substantially pure triethylenetetramine dihydrochloride or tetrahydrochloride. In another aspect the method employs a pharmaceutical composition comprising substantially pure triethylenetetramine dihydrochloride or tetrahydrochloride and a pharmaceutically acceptable excipient.

In one aspect of the invention, the method employs a crystalline form of triethylenetetramine disuccinate or a hydrochloride salt of triethylenetetramine. In another aspect of the invention, the method employs triethylenetetramine disuccinate anhydrate or a hydrochloride salt of triethylenetetramine anhydrate.

In certain embodiments, the triethylenetetramine succinate salt is a triethylenetetramine disuccinate polymorph. In certain embodiments, the triethylenetetramine hydrochloride salt is a triethylenetetramine hydrochloride polymorph.

In one aspect, the invention comprises a method of treatment for the prevention or amelioration of coronavirus infection in a subject, e.g., SARS-CoV-2 infection, and COVID-19 disease, the method comprising administering to said subject a therapeutically effective amount of compound selected from the group consisting of a trientine, a succinic acid addition salt of triethylenetetramine, a hydrochloric acid addition salt of triethylenetetramine, and pharmaceutically acceptable salts of D-penicillamine, N-acetylpenicillamine, tetrathiomolybdate, ammonium tetrathiomolybdate, and choline tetrathiomolybdate.

In one aspect of the invention, the administration of the copper-depriving agent provides a prophylactic effect against viral infection for about 8 to about 24 hours. In one aspect, the administration provides a prophylactic effect for about a 24-hour period. In another aspect, the administration provides a prophylactic effect for about a 24-48 hour period. In still another aspect, the administration provides a prophylactic effect for about 48 to about 72 hours, or more.

In another aspect of the invention, the method comprises or consists essentially of administration of a nanoemulsion with a copper-depriving agent that persists in the nasal mucosa or skin for about 24 hours or more.

In another aspect of the invention, the method comprises or consists essentially of the use of a compound (a) which itself in vivo or (b) which has at least one metabolite in vivo that is (i) a copper chelator or (ii) otherwise reduces available copper values, for the production of a pharmaceutical composition or dosage unit able to reduce the level of copper in a mammal, or able to reduce the level of copper available to the coronavirus while maintaining copper levels with about 70 to about 100% of normal in the subject, thereby eliciting by a lowering of copper values in a mammalian patient and/or reducing the level of copper available to the coronavirus to prevent, reduce or treat a coronavirus infection, e.g., to prevent or treat a SARS-CoV-2 infection, and COVID-19 disease.

Copper-depriving compounds may be administered at dosages or a dosage to provide, if parenteral, at least about 120 mg/day in a human patient, and if oral, at least about 1200 mg/day in a human patient. Some oral doses of copper-depriving compounds may be administered at about 1200 to about 2400 mg/day. The total dosage may be given in single or divided dosage units (e.g., BID, TID, QID), and preferably maintain normal urine and/or plasma copper levels in a subject, or levels that do not fall below about 70% to 75% of normal. BID is presently preferred.

Other doses to treat human patients may range from about 10 mg to about 2000 mg/day of a virus copper-depriving compound. A typical dose may be about 100 mg to about 1500 mg/day of the compound. Other doses are from about 300 to about 2400 milligrams per day of the compound. Other doses include about 500 mg to about 1200 mg/day of the compound. Other doses are from about 600 to about 2400 milligrams per day of the compound. A dose may be administered once a day (QD), twice per day (BID), or more frequently, depending on the pharmacokinetic and pharmacodynamic properties, including absorption, distribution, metabolism, and excretion of the particular compound. In addition, toxicity factors may influence the dosage and administration regimen. When administered orally, the pill, capsule, or tablet may be ingested daily or less frequently for a specified period of time. The regimen may be repeated for a number of cycles of therapy.

In another aspect of the invention for treatment or prevention of coronavirus infections that require copper modulation an appropriate dosage level will generally be about 0.5 to about 50 mg or 100 mg per kg patient body weight per day which can be administered in single or multiple doses. Preferably, the dosage level will be about 1 to about 25 mg/kg per day; more preferably about 5 to about 10 mg/kg per day. A suitable dosage level may be about 0.5 to 25 mg/kg per day, about 1 to 10 mg/kg per day, or about 1 to 5 mg/kg per day. Within this range the dosage may be about 0.5 to about 1.0, 0.5 to 2.5 or 0.5 to 5 mg/kg per day. For oral administration, the compositions are preferably provided in the form of tablets containing about 100 to 1000 milligrams of the active ingredient, particularly about 100, 150, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, preferably once or twice per day.

Other exemplary doses include doses in the range of about 1 to 20 mg of active agent per kilogram of subject's body weight per day, preferably about 7 to about 18 mg/kg/day, or about 8 to 17 mg/kg/day, or about 10 to 15 mg/kg/day. The total dosage may be given in single or divided dosage units (e.g., preferably BID, but also TID or QID).

In any of the procedures described and/or claimed herein, the copper-depriving compound may be a copper chelator in which the dosage regimen to be given to the subject will not chelate copper and reduce it down to a depletion state or to an otherwise dangerously low level in the subject. In one embodiment, a copper-depriving compound, such as a chelator or copper transporter knockdown or other inhibitor, is administered at a dosage regimen less than that which would have the effect of decreasing the copper levels of that patient to abnormal, or less than about 70 to about 75% of normal. The administration is at a dosage regimen (whether dependent upon dosage unit(s) and/or frequency) that does not or will not reduce a patient of normal copper levels to a deficiency state.

Dosage forms useful herein include any appropriate dosage form known in the art to be suitable for pharmaceutical formulation of compounds suitable for administration to mammals particularly humans, particularly (although not solely) those suitable for stabilization in solutions, capsules or sprays comprising therapeutic compounds for administration to humans. The dosage forms of the invention thus include any appropriate dosage form now known or later discovered in the art to be suitable for pharmaceutical formulation of compounds suitable for administration to humans. One example is oral delivery forms of tablet, capsule, lozenge, or the like form, or any liquid form such as syrups, aqueous solutions, emulsion and the like, capable of protecting the compound from degradation prior to eliciting an effect, for example, in the alimentary canal if an oral dosage form. Examples of dosage forms for transdermal delivery include transdermal patches, transdermal bandages, and the like. Included within the topical dosage forms are any lotion, stick, spray, ointment, paste, cream, gel, etc., whether applied directly to the skin or via an intermediary such as a pad, patch or the like. Examples of dosage forms for suppository delivery include any solid or other dosage form to be inserted into a bodily orifice (particularly those inserted rectally, vaginally and urethrally). Examples of dosage units for transmucosal delivery include depositories, solutions for enemas, pessaries, tampons, creams, gels, pastes, foams, nebulized solutions, powders and similar formulations containing in addition to the active ingredients such carriers as are known in the art to be appropriate. Examples of dosage units for depot administration include pellets or small cylinders of active agent or solid forms wherein the active agent is entrapped in a matrix of biodegradable polymers, microemulsions, liposomes or is microencapsulated. Examples of implantable infusion devices include any solid form in which the active agent is encapsulated within or dispersed throughout a biodegradable polymer or synthetic, polymer such as silicone, silicone rubber, silastic or similar polymer. Alternatively, dosage forms for infusion devices may employ liposome delivery systems.

Depending on the subject's condition, the compounds of the present invention may be administered by oral, parenteral (for example, intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. Also useful are intraesophageal, intragastric, intraduodenal and intrajejunal administration via nasogastric, nasoduodenal and intrastomal routes, for example. The pharmaceutical composition and method of the present invention may further comprise other therapeutically active compounds as noted herein which are usually applied in the prophylaxis or treatment of the above-mentioned infections. As noted, in various embodiments of methods of the invention, administration of the copper-depriving compound is by endotracheal, endosinusial, intrabronchial, intracavernous, intrasinal, intrapulmonary or transmucosal administration.

Nasal or endosinusial or intrapulmonary administration, for example, may be accomplished using a variety of means, such as by use of a nanoemulsion comprising droplets having, for example, an average diameter less than about 1000 nm, and wherein the nanoemulsion comprises, consists essentially of, or consists of: (a) an aqueous phase; (b) an oil phase comprising at least one oil and optionally at least one organic solvent; and (c) at least one surfactant.

In some embodiments, following nasal administration, endosinusial administration, or administration to the lung, the compounds of the invention (in the form of, e.g., nanoemulsion droplets or liposomes), persist in the nasal or lung mucosa for about 24 hours or more.

In some embodiments, administration increases the chance of survival following exposure to a coronavirus. In some embodiments, administration reduces the colonization of coronavirus in the nose or on the skin. In some embodiments, administration reduces the risk of transmission of coronavirus. In some embodiments, survival is increased by about 10%, about 20%, about 30%, about 40%, about 50%, about 60%, about 70%, about 80%, about 90%, or about 100%.

In some embodiments, the coronavirus comprises, consists essentially of, or consists of human coronavirus 229E, human coronavirus OC43, SARS-CoV, HCoV-NL63, HKU1, MERS-CoV, or SARS-CoV-2. In some embodiments, the risk of infection to be prevented or reduced is by coronavirus disease 2019 (COVID-19). SARS-CoV-2 is a positive-sense and single-stranded RNA virus of zoonotic origin belonging to Betacoronavirus lineage B. In some embodiments, the coronavirus comprises, consists essentially of, or consists of a viral particle translated from a polynucleotide comprising a SARS-CoV-2 or any copper-requiring strain or variant or mutation thereof (including synonymous mutations and missense mutations, mutations not in genes for structural proteins of SARS-CoV-2 and those that do not result in changes in the amino acid sequences of SARS-CoV-2 structural proteins), including evolved genomic alterations and those showing genomic divergence across successive generations (see, e.g., Yellapu, N. K., et. al, Evolutionary Analysis of Severe Acute Respiratory Syndrome Coronavirus 2 (SARS-CoV-2) Reveals Genomic Divergence with Implications for Universal Vaccine Efficacy Vaccines 2020, 8(4), 591 (8 Oct. 2020); a fragment thereof coding for or included within a viable or infectious viral particle susceptible to copper deprivation; a polynucleotide comprising SARS-CoV-2 (GenBank accession number NC_0455122), or a copper-requiring strain or variant or mutation thereof, an infectious fragment thereof coding for or included within a viable or infectious viral particle susceptible to copper deprivation, or a copper-requiring infectious polynucleotide having at least 80% sequence identity to the polynucleotide comprising SARS-CoV-2, including, for example, 80% sequence identity to a polynucleotide according to GenBank accession number NC_0455122). A reference genome in FASTA format is provided for SARS-CoV-2, “Severe acute respiratory syndrome coronavirus 2 isolate Wuhan-Hu-1, complete genome,” having the accession ID of NC_045512.2. See, e.g., Wang, X., et al., Nosocomial outbreak of COVID-19 pneumonia in Wuhan, China. Eur Respir J. 2020 June; 55(6): 2000544 (whole-genome sequencing of 25 infected health care workers). SARS-CoV-2 sequence data are available from various sources, including deposits in the National Center for Biotechnology Information Sequence Read Archive, which are also incorporated herein by reference, as if fully set out herein.

In some embodiments of the invention, the coronavirus comprises, consists essentially of, or consists of coronavirus variants. In some embodiments, of the invention, the coronavirus comprises, consists essentially of, or consists of COVID-19 variants, including the so-called United Kingdom or UK variant (named B.1.1.7), the so-called South Africa variant, named B.1.351 (which emerged independently of B.1.1.7 but shares some mutations with B.1.1.7), the so-called Brazil variant, named P.1, and the so-called Southern California variant, named CAL. 20C.

In some embodiments, the method comprises administering a tablet or capsule to a subject. In other embodiments, administering comprises or consists essentially of or consists of administration of a nasal spray, medicated nasal swab, medicated wipe or aerosol comprising the composition to the subject's nasal vestibule or nasal passages. In some embodiments, the subject is exposed to or is anticipated to be exposed to an individual with one or more symptoms selected from the group consisting of fever, cough, shortness of breath, diarrhea, sneezing, runny nose, and sore throat.

In some embodiments, the subject is a healthcare worker, elderly person, frequent traveler, military personnel, caregiver, or a subject with a preexisting condition(s) that result(s) in increased risk of mortality with infection.

In some embodiments, the preexisting condition comprises age over 60-65 or 70 years or greater, diabetes (especially type 2 diabetes), heart disease or obesity. In other embodiments, the preexisting condition comprises people of any age with other underlying medical conditions are at increased risk for severe illness from SARS-CoV-2, including cancer, chronic kidney disease, COPD (chronic obstructive pulmonary disease), hypertension, obesity, immunocompromised state (weakened immune system) from any cause, including, for example, chemotherapy, Crohn's Disease, IBD, etc., serious heart diseases such as heart failure, coronary artery disease or cardiomyopathies, sickle cell disease, and anemia.

People with the following conditions may also be at an increased risk for severe illness from SARS-CoV-2: asthma (moderate to severe), cerebrovascular disease, cystic fibrosis, hypertension, neurologic conditions (such as dementia and Parkinson's and Alzheimer's), liver disease, HIV, use of corticosteroids or other immune-weakening medicines, pregnancy, pulmonary fibrosis (having damaged or scarred lung tissue), smoking, thalassemia.

A high frequency of sensitively of coronaviruses such as SARS, MERS, and including SARS-CoV-2 to copper chelators can be confirmed. It has also been determined as described and claimed herein that treatment with specific copper chelators and other agents that decrease copper values and, preferably, do not lead to depletion states of other transition metals (e.g., iron, zinc and manganese), or essential metals, will benefit a significant number and spectrum of the population, including for those diseases, disorders, and/or conditions described above.

A preferred pharmaceutical composition for use in the methods of the invention comprises or consists essentially of or consists of an agent substantially pure triethylenetetramine disuccinate. Another preferred composition is substantially pure triethylenetetramine disuccinate anhydrate. Another preferred composition is a composition that comprises or consists essentially of or consists of an agent a substantially pure triethylenetetramine disuccinate crystal having alternating layers of triethylenetetramine molecules and succinate molecules.

Without wishing to be bound by any particular theory or mechanism, copper values, particularly, e.g., copper(I) and/or copper(II), will be required for coronavirus survival and replication. Without wishing to be bound by any particular theory or mechanism, it is believed that reduction in available free copper or intracellular copper will interrupt the coronavirus lifecycle and/or its infectivity. This is irrespective of the infection status of the patient and is thus applicable whether the subject tests positive for a coronavirus, has been exposed to or is anticipated to be exposed to an individual with a coronavirus, e.g., SARS-CoV-2. The present invention provides for a desired reduction in available free copper values or intracellular copper of coronavirus host cells as a preventive and/or treatment approach.

Evaluation of therapy may be accomplished not only by viral testing, but by reference to available copper values in mammals (including human beings), those mammalian patients with a copper level that is “elevated” beyond that of the general population of such mammals can be identified. Reference herein to “elevated” in relation to the presence of copper values will include humans having at least about 10 mcg free copper/dL of serum when measured. A measurement of free copper equal to total plasma copper minus ceruloplasmin-bound copper can be made using various procedures. A preferred procedure is disclosed in the Merck & Co datasheet (www.Merck.com) for SYPRINE (trientine hydrochloride) capsules, a compound used for treatment of Wilson's Disease, in which a 24-hour urinary copper analysis is undertaken to determine free cooper in the serum by calculating the difference between quantitatively determined total copper and ceruloplasmin-copper. SYPRINE, also referred to as N,N′-bis (2-aminoethyl)-1,2-ethanediamine dihydrochloride, has the structural formula: NH₂(CH₂)₂NH(CH₂)₂NH(CH₂)₂NH₂.2HCl.

Copper chelating agents, copper sequestering agents, copper depriving agents, copper-removing agents, alone or together with other agents, including antivirals and anti-inflammatories, may be administered alone or in combination with one or more additional ingredients and may be formulated into pharmaceutical compositions including one or more pharmaceutically acceptable excipients, diluents and/or carriers.

In some embodiments, administering further comprises administration of one or more antiviral drugs. In some embodiments, administering further comprises administration of one or more antiviral drugs selected from the group consisting of chloroquine, hydroxychloroquine, darunavir, galidesivir, interferon beta, lopinavir, ritonavir, remdesivir, and triazavirin. Others are described herein. In one embodiment, the interferon is selected from the group consisting of interferon β-1b, interferon α-n1, interferon α-n3, pegylated interferon β-1b, pegylated interferon α-n1, pegylated interferon α-n3 and human leukocyte interferon α.

Other embodiments of the invention include an article of manufacture comprising a single dose capsule or tablet containing a single fixed dose of triethylenetetramine disuccinate, wherein the fixed dose is selected from the group consisting of about 350 mg, about 584 mg and about 701 mg of triethylenetetramine disuccinate. In some embodiments, the article of manufacture of claim further comprising a package insert instructing the user to administer the fixed dose to a patient with a coronavirus disease treatable with a copper chelator. In one embodiment of the article of manufacture, the coronavirus disease treatable with a copper chelator is characterized by excess copper. In another embodiment, the article of manufacture comprises or consists essentially of a number of fixed dose capsules equal to one or more daily doses of triethylenetetramine disuccinate, wherein the daily dose is selected from the group consisting of from about 1050 mg per day to about 2300 mg per day, about 1400 mg per day to about 3500 mg per day, about 2300 mg per day to about 2800 mg per day, and about 2800 mg per day to about 5600 mg per day of triethylenetetramine disuccinate and, optionally, wherein the wherein the fixed dose is selected from the group consisting of about 350 mg, about 400 mg, about 500 mg, about 584 mg about 600 mg and about 701 mg of triethylenetetramine disuccinate. In some embodiments of the article of manufacture, the triethylenetetramine disuccinate has a purity of at least about 95%, at least about 99%, or is pure. In some embodiments of the article of manufacture, the triethylenetetramine disuccinate is a crystalline form of triethylenetetramine disuccinate. In other embodiments of the article of manufacture, the triethylenetetramine disuccinate is a triethylenetetramine disuccinate anhydrate. In some embodiments of the article of manufacture, the triethylenetetramine disuccinate is a triethylenetetramine disuccinate polymorph.

In some embodiments of the article of manufacture, the fixed dose of triethylenetetramine disuccinate is about 350 mg, about 584 mg, about 600 mg, or about 700 mg. In some embodiments of the article of manufacture, the triethylenetetramine disuccinate is in the form of a capsule or tablet. In some embodiments of the article of manufacture, the triethylenetetramine disuccinate capsule or tablet is packaged in a blister pack, or a bottle. In some embodiments of the article of manufacture, the triethylenetetramine disuccinate capsule or tablet is formulated to provide for a delayed release. In some embodiments of the article of manufacture, the triethylenetetramine disuccinate capsule or tablet is formulated to provide for a sustained release. In other embodiments of the article of manufacture, the triethylenetetramine disuccinate capsule or tablet is formulated in combination with a pharmacokinetic enhancer (PKE) that provides for improved absorption of the triethylenetetramine disuccinate.

In methods of the invention for treating or managing a subject with (or suspected of having) a coronavirus disease treatable with a copper chelator, the method comprising administering to said subject a fixed dose of triethylenetetramine disuccinate, wherein the fixed dose ranging from about 350 to about 700 milligrams. In other embodiments of the methods, the fixed dose of triethylenetetramine disuccinate is about 350 mg, 400 mg, about 500 mg, about 600 mg or about 700 mg. In still other embodiments of the methods, fixed doses of triethylenetetramine disuccinate are administered to the subject in an amount ranging from about 1050 mg per day to about 2300 mg per day, about 1400 mg per day to about 3500 mg per day, about 2300 mg per day to about 2800 mg per day, about 2400 mg per day to about 3000 mg per day, and about 2800 mg per day to about 5600 mg per day. In some embodiments of the method, the subject is a human. In other embodiments, one or more symptoms or diagnostic markers of the coronavirus disease is/are reduced. In some embodiments of the method, using for example a fixed dose of triethylenetetramine disuccinate is in the form of a capsule or tablet for oral administration, the fixed dose of triethylenetetramine disuccinate lowers copper values content and/or reduces intracellular copper in the subject, the fixed dose of triethylenetetramine disuccinate reduces total copper, and/or the fixed dose of triethylenetetramine disuccinate reduces intracellular copper.

In another embodiment of the invention, the article of manufacture comprises or consists essentially of triethylenetetramine disuccinate and an inhibitor of N-acetylaminotransferase. In one embodiment, the inhibitor of N-acetylaminotransferase is an inhibitor of spermine/spermidine N-acetyltransferase (SSAT1). In another embodiment, the inhibitor of N-acetylaminotransferase is an inhibitor of spermine/spermidine N-acetyltransferase (SSAT2).

In another embodiment of the invention, the article of manufacture comprises or consists essentially of triethylenetetramine disuccinate and a promoter of polyamine membrane transport including bergamottin, maringenin, quercetin, other psoralens, piperine, or tetrahydro-piperine that act as enhancers of membrane permeability for increased absorption.

In another embodiment of the invention, fixed dose triethylenetetramine disuccinate capsule or tablet has a shelf-life term of at least about 12 months at room temperature. In one embodiment, the article of manufacture has a minimum purity of the triethylenetetramine disuccinate over said shelf-life term is least about 98.5% with no degradation product above about 0.5% and no new, unidentified impurities above about 0.1%. in another embodiment of the article of manufacture, the shelf-life term is about 12 months.

Both the foregoing summary and the following detailed description are exemplary and explanatory. They are intended to provide further details of the invention but are not to be construed as limiting. Other objects, advantages, and novel features will be apparent to those skilled in the art from the following detailed description of the invention.

### BRIEF DESCRIPTION OF DRAWINGS

FIG. 1 is a schematic diagram of the final PK/PD model used to describe TETA, MAT, and DAT plasma concentrations and urinary copper excretion versus time in Example 14. Symbols are defined in the List of Abbreviations and in Table 8.

### DETAILED DESCRIPTION

Copper is an essential trace nutrient in eukaryotic cells. While the essential role of copper in eukaryotic cellular physiology is known, it has not been recognized as important in the context of coronavirus infection, including in the replication of the coronaviruses that cause COVID-19 disease.

The treatment methods described and claimed herein suppress ssRNA viral replication within mammalian systems including humans; in particular in those humans/patients where there is evidence for viral replication, namely in those with one or more positive tests for the coronavirus, including in the replication of coronaviruses that cause COVID-19 disease. Positive tests can be for example those where viral proteins are detected by antibody tests, or those where the evidence is based on the polymerase chain reaction (PCR) test, particularly those tests performed by reverse transcriptase-PCR (RT-PCR test), or by any future method of testing.

Copper chelators are copper-depriving agents. Copper-depriving compounds include, for example, the copper chelator triethylenetetramine (TETA) and its salts. A preferred copper-depriving compound is triethylenetetramine disuccinate. We have shown in Example 1 that, when administered to experimental animals in vivo, triethylenetetramine disuccinate enters organs including the upper respiratory tract, the lungs, and the heart. These are sites of coronavirus replication and are the same organs that are attacked by coronavirus infection in humans, including the coronaviruses leading to COVID-19 disease. Example 1 describes a quantitative in vivo study on the tissue distribution of the copper-depriving compound triethylenetetramine disuccinate following oral administration to male albino and male pigmented rats. Significant tissue penetration was found throughout 42 different body tissues, including the heart, lung and nasal tissue in both species. In the male pigmented rat, maximum tissue concentrations of radioactivity were evenly distributed between one-hour and eight-hour time points. Highest levels of radioactivity were seen in the various tissues that included the lung at one hour post-dose, with penetration to the lung continuing for a full eight hours. This is significant for a drug used to prevent and treat a respiratory virus, like the coronavirus. Evaluation of the use of copper-depriving compounds in treating coronavirus infection, and the requirements for copper in coronavirus replication, is described in Examples 2-12. Novel dosing regimens for triethylenetetramine disuccinate are described herein, and in Examples 13 and 14. We demonstrated in the Example 15 study that triethylenetetramine disuccinate will have good absorption in humans (estimated at approximately 70%).

Optimal fixed dosing of triethylenetetramine disuccinate for the treatment of diseases has been discovered and is provided herein following the studies described in the below Examples, including the Example 1 in vivo distribution study and the human clinical study results described, interpreted and evaluated in Examples 13 and 14, which revealed unexpected findings including those on the copper chelation activity and breakdown of triethylenetetramine disuccinate into major metabolites, average plasma concentration, full body tissue distribution, tissue:blood ratios following oral administration, and triethylenetetramine disuccinate bioavailability, amongst other things.

Example 1 describes a quantitative in vivo study on the tissue distribution of the labelled copper-depriving compound triethylenetetramine disuccinate following oral administration to male albino and male pigmented rats. Significant tissue penetration was found throughout 42 different body tissues, including the brain, heart, lung and liver in both species. In the male pigmented rat, maximum tissue concentrations of radioactivity were evenly distributed between the 1 h and 8 h time points. Highest levels of radioactivity were seen in the various tissues that included the lung at 1 hr post-dose, with penetration to the lung continuing for a full 8 hours. At 24 h post-dose elimination was on-going in the male pigmented rat with approximately half of the measured tissues having levels of radioactivity below the limit of quantification. At 72 h post-dose, elimination of radioactivity in the male pigmented rat was almost complete with approximately 65% of tissues below the limit of quantification.

Example 13 describes human population pharmacokinetic and pharmacodynamic modeling of triethylenetetramine, its two major metabolites, and copper excretion after oral 2-way crossover administration of triethylenetetramine disuccinate and triethylenetetramine dihydrochloride in a clinical study to healthy adult volunteers, revealing, amongst other things, the bioavailability of triethylenetetramine disuccinate in humans. The population PK analysis encompassed samples from this study where each subject received triethylenetetramine disuccinate and triethylenetetramine dihydrochloride (Syprine®) in a double-blind, dose escalation, 2-way crossover design.

Example 14 describes further analyses of data obtained in the Example 13 study comparing triethylenetetramine disuccinate and triethylenetetramine dihydrochloride (Syprine®). The Example 13 study resulted in the discovery that administration of triethylenetetramine as the disuccinate salt results in lower exposure indices (Cₘₐₓ and AUC) of triethylenetetramine and its metabolites. The modeling in Example 14 compared the absorption kinetics and provided a more global assessment of relative bioavailability of the two salt forms in the context of the Example 2 study design. The Example 14 analysis applied a model-based population analysis to the data in order to obtain an integrated assessment of the pharmacokinetics of triethylenetetramine and its two major metabolites (monoacetylated (MAT) and diacetylated (DAT forms) and to further assess the pharmacodynamics of urinary excretion of copper, to consider potential covariates with the PK/PD parameters such as sex, age and dose, and in comparing the PK/PD of Syprine® and triethylenetetramine disuccinate from the Example 13 bioequivalency study, particularly in regard to bioavailability. Example 15 demonstrates that triethylenetetramine disuccinate will have good absorption in humans (estimated at approximately 70%).

Triethylenetetramine dihydrochloride is a copper chelator that was approved by the FDA for the second line treatment of Wilson's Disease. It is available in Europe in 300 mg capsules and in general two capsules are administered BID (1200 mg per day total) to treat Wilson's Disease. Triethylenetetramine dihydrochloride (Syprine®) is available in the United States in 250 mg capsules and in general two capsules are administered BID (1000 mg per day total) to treat Wilson's Disease. Systemic evaluation of Syprine® dose and/or interval between doses has not been done. However, on limited clinical experience, the recommended initial dose of Syprine® in the United States is 500-750 mg/day for pediatric patients and 750-1250 mg/day (up to 2000 mg/day) for adults given in divided doses two, three or four times daily.

Triethylenetetramine disuccinate is an alternative, superior salt form of triethylenetetramine, but its target dosing is unknown, and unknowable from the prior art. We have discovered that in order to duplicate the bioavailability of triethylenetetramine in 300 mg triethylenetetramine dihydrochloride, about 701 mg of triethylenetetramine disuccinate is required. In order to duplicate the bioavailability of triethylenetetramine in 250 mg triethylenetetramine dihydrochloride, we discovered that about 584 mg of triethylenetetramine disuccinate is required. In order to duplicate the bioavailability of triethylenetetramine in 250 mg triethylenetetramine tetrahydrochloride (which is bioequivalent to the dihydrochloride salt), we discovered that about 350 mg of triethylenetetramine disuccinate is required.

Thus, we not only discovered that fixed doses comprising or consisting essentially of about 701 mg of triethylenetetramine disuccinate and about 584 mg of triethylenetetramine disuccinate, and about 350 mg of triethylenetetramine disuccinate are optimal for dosing this salt form, but that per day doses of about 2336 mg and 2804 mg of triethylenetetramine disuccinate are optimal for treatment of Wilson's disease and other copper disorders, based on 1000 mg/day and 1200 mg/day dosing respectively.

Using the above per day triethylenetetramine dihydrochloride pediatric and adult dosing ranges of 500-750 mg for children and 750-1250 mg (and up to 2000 mg/day) for adults, the per day triethylenetetramine disuccinate dose ranges are from about 1168 mg to about 1752 mg for children and from about 1752 mg to about 2920 mg (and up to 4672 mg/day) for adults. Doses are increased if the clinical response not adequate or free serum copper are persistently >20 mcg/dL, and long-term maintenance doses are reassessed every 6-12 months.

Another approved daily dose of trientine dihydrochloride is 1200-2400 mg/day in 2-4 divided doses for adults, and a lower dose, typically 600-1500 mg/day, depending on age and body weight, for children, also typically given in divided doses. Based on the discoveries herein, the superior triethylenetetramine disuccinate salt would be dosed at about 2803 mg/day to about 5606 mg/day for adults, and about 1402 mg/day to about 3504 mg/day, depending on age and body weight, for children, all typically given in divided doses.

Cuprior® (triethylenetetramine tetrahydrochloride) is also indicated for the treatment of Wilson's disease in adults, adolescents and children ≥5 years intolerant to D-penicillamine therapy and is sold as 150 mg tablets. The approved and recommended Cuprior® dosing regimen for adults is between 450 mg and 975 mg (3 to 6½ tablets) per day in 2 to 4 divided doses. The triethylenetetramine disuccinate dosing regimen for adults would be between about 1051 mg and about 2278 mg per day (typically using a 350-350.4 mg fixed dose, which corresponds to the 150 mg triethylenetetramine tetrahydrochloride tablet). The starting dose in pediatrics is lower than for adults and depends on age and body weight. In general, the Cuprior® dose for children is usually between 225 mg and 600 mg per day (1½ to 4 tablets) in 2 to 4 divided doses. The triethylenetetramine disuccinate dosing regimen for children would be between about 525 mg and about 1400 mg per day.

We further discovered that other fixed doses of triethylenetetramine disuccinate for optimal dosing and bioavailability are about 350 mg, about 400 mg, about 500 mg, about 600 mg and about 700 mg of triethylenetetramine disuccinate, including fixed doses of about 350.4 mg, 584 mg and about 701 mg of triethylenetetramine disuccinate. Exemplary effective amounts are described herein, and include doses in the range of from about 2300 mg per day to about 2800 mg per day given as multiple fixed doses of triethylenetetramine disuccinate comprising or consisting essentially of about 350 mg, 400 mg, about 500 mg, about 600 mg and/or about 700 mg, for example. Other fixed doses of triethylenetetramine disuccinate are given to equal about 1050 mg/day to about 2300 mg/day, about 1400 mg/day to about 3500 mg/day, about 2400 mg/day to about 3000 mg/day, and about 2800 mg/day to about 5600 mg/day.

By way of example, four 350 mg triethylenetetramine disuccinate capsules given BID would equal 2800 mg per day (roughly equivalent to 2804 mg per day, which is the triethylenetetramine disuccinate dose that we discovered is bioequivalent to the 1200 mg per day triethylenetetramine dihydrochloride administered in Europe for treating Wilson's Disease). Three 400 mg triethylenetetramine disuccinate capsules, for example, given BID would equal 2400 mg per day (roughly equivalent to 2337 mg per day, which is the triethylenetetramine disuccinate dose that we discovered is bioequivalent to the 1000 mg per day triethylenetetramine dihydrochloride administered in the United States for treating Wilson's Disease).

Three 500 mg triethylenetetramine disuccinate fixed dose tablets/capsules, etc., for example, given BID would equal 3000 mg per day, roughly equivalent to the 2804 mg per day triethylenetetramine disuccinate bioequivalent dose we discovered. Four 600 mg triethylenetetramine disuccinate fixed dose tablets/capsules, etc., for example, given BID equals 2400 mg per day, which is roughly equivalent to the 2337 mg per day triethylenetetramine disuccinate bioequivalent dose we discovered. Two 700 mg triethylenetetramine disuccinate fixed dose tablets/capsules, etc., for example, given BID would equal 2800 mg per day, which is roughly equivalent to the 2804 mg per day triethylenetetramine disuccinate bioequivalent dose.

Other convenient fixed dose amounts of triethylenetetramine disuccinate can be calculated and manufactured to provide daily bioequivalent doses, such as about 2804 mg per day and about 2337 mg per day. For example, five 280 mg triethylenetetramine disuccinate doses given BID can be used to provide 2800 mg per day. Also, by way of example, four 290 mg triethylenetetramine disuccinate doses given BID can be used to provide 2320 mg per day.

Fixed doses of about 350 mg, about 584 mg and about 701 mg of triethylenetetramine disuccinate may also be given as two doses BID to equal per day doses of about 1400 mg, about 2336 mg and about 2804 mg of triethylenetetramine disuccinate, respectively. These and other fixed doses and total per day dose amounts described herein may be used to treat Wilson's disease and other copper disorders, including those described or referenced herein.

In general, the dosing is between about 2.336 and 2.337 mg of triethylenetetramine disuccinate for every milligram of triethylenetetramine dihydrochloride or triethylenetetramine tetrahydrochloride.

By way of example, we have also discovered that 2804 mg triethylenetetramine disuccinate per day, given as two 700 mg capsules administered twice daily, for example, would be expected to produce a significant cupruresis effect throughout the dosing interval with minimal side effects and negligible adverse effects on serum copper levels or other laboratory test parameters for treatment of heart disease, including, for example, heart disease in type 2 diabetic patients, in whom cardiomyopathy (e.g., elevated left ventricular mass) may also be treated with these dose amounts. Six months of treatment of elevated left ventricular mass with triethylenetetramine disuccinate dosed as described to provide about 2800 mg per day will cause elevated left ventricular mass to decline significantly toward normal.

We further discovered that fixed doses of triethylenetetramine disuccinate for optimal dosing and bioavailability given as multiple fixed doses of triethylenetetramine disuccinate comprising or consisting essentially of about 350 mg, 400 mg, about 500 mg, about 584, about 600 mg and/or about 700 or 701 mg will be useful for treating a copper-related disease, disorder or condition, as described herein.

In one aspect, the invention relates to newly discovered fixed dose amounts of triethylenetetramine disuccinate, formulations thereof, and their use for the treatment, prevention or amelioration of diseases, conditions and disorders treatable with copper chelators.

In certain embodiments, triethylenetetramine disuccinate is administered at an initial dose (or loading dose) followed by a maintenance dose, wherein the loading dose is about or at least 1.5 times greater, about or at least 2 times greater, about or at least 2.5 times greater, or about or at least 3 times greater than the maintenance dose. The maintenance dose may be, for example, about 350 mg, 400 mg, about 500 mg, about 584 mg, about 600 mg and/or about 700 or 701 mg, from 1-4 times per day. In one embodiment, the loading dose is administered once, twice, three, four, or five times before the first maintenance dose, and may be given once, twice, three times or four times a day.

Thus, by way of example, in one embodiment, for a 2337 mg per day triethylenetetramine disuccinate loading dose regimen, triethylenetetramine disuccinate is administered at a daily loading dose (which can be provided in one or several dosages throughout the day) of at least about 3505 mg (1.5×), at least about 4674 mg (2×), at least about 5842 mg (2.5×), or at least about 7001 mg (3×). In one embodiment, the triethylenetetramine disuccinate loading dose is administered in two doses a day, and optionally over 1, 2, 3, 4 or 5 or more days. Other triethylenetetramine disuccinate loading doses are calculated accordingly, based on triethylenetetramine disuccinate maintenance doses given daily or in other frequencies, such as, for example, 2804 or other maintenance doses given daily.

In one embodiment, the triethylenetetramine disuccinate fixed described herein doses are administered twice per day (BID) to provide the desired per day dosing. In another embodiment, the triethylenetetramine disuccinate fixed doses are administered three times per day (TID) to provide desired per day dosing. In a still further embodiment, the triethylenetetramine disuccinate fixed doses are administered four times per day (QID) to provide desired per day dosing.

Importantly, the crystalline anhydrous form of the triethylenetetramine disuccinate article of manufacture described herein has a shelf-life of at least about 12 months (and up to five years) at room temperature, without significant degradation of the triethylenetetramine disuccinate API and remains within impurity specifications for the triethylenetetramine disuccinate drug substance. In one embodiment, the term “without significant degradation” means that the purity of the triethylenetetramine disuccinate is at least about 98.5% with no degradation product above about 0.5% and no new, unidentified impurities above about 0.1% for at least about 12 months.

We have discovered that triethylenetetramine disuccinate 1200 mg/day, given as 600 mg twice daily, would be expected to produce a significant cupruresis effect throughout the dosing interval with minimal side effects and negligible adverse effects on serum copper levels or other laboratory test parameters. Based on Examples 13 and 14, we further discovered that fixed doses of triethylenetetramine disuccinate for optimal dosing and bioavailability are about 350 mg, 400 mg, about 500 mg, about 600 mg and about 700 mg of triethylenetetramine disuccinate. Exemplary effective amounts are described herein, and include doses in the range of from about 2400 mg per day to about 3000 mg per day given as multiple fixed doses of triethylenetetramine disuccinate comprising or consisting essentially of about 350 mg, 400 mg, about 500 mg, about 600 mg and/or about 700 mg. These are the preferred doses of triethylenetetramine disuccinate, and the preferred fixed doses used in accordance with the coronavirus treatment methods of the invention.

In one aspect, the invention relates to newly discovered fixed dose amounts of triethylenetetramine disuccinate, and formulations thereof. In another aspect, the invention relates to the use of these fixed dose triethylenetetramine disuccinate formulations for the treatment, prevention or amelioration of coronavirus diseases, disorders and conditions, including active infections.

Copper chelators, including triethylenetetramines such as triethylenetetramine disuccinate and triethylenetetramine dihydrochloride, D-penicillamine, and tetrathiomolybdate salts (for example ammonium tetrathiomolybdate and bis-choline tetrathiomolybdate) extract copper from the tissues of experimental animals including rats and dogs, thereby lowering the availability of copper to viruses infecting treated cells. Other known, unknown, or unrecognized agents that have copper-binding properties, whether or not by chelation, and whether or not normally used for this purpose, will also be useful in the methods described and claimed herein, as will copper transporter antagonists or other compounds that inhibit copper transport and use in viral host cells.

Some copper-depriving agents work by different mechanisms of action. For example, copper chelators like the triethylenetetramines act on differing ions compared with D-penicillamine. The former act as a copper(II) whereas the latter acts as a mixed copper-(I)/copper-(II) chelator. Copper (I) is essential to intracellular health.

The presence of triethylenetetramine disuccinate in the cells of treated rats provides substantive evidence that the chelator can prevent coronaviruses from obtaining sufficient copper to support replication and thereby act as an anti-viral agent for or in relevant tissues, such as the upper respiratory tract, the lungs, and the heart.

Some preferred copper-depriving compounds are triethylenetetramine disuccinate and triethylenetetramine dihydrochloride, which have been shown to extract copper from the body of normal humans in a dose-dependent manner. These drugs can thus act as anti-viral agents in humans for or in relevant tissues, including the upper respiratory tract, the lungs, and the heart.

Triethylenetetramine disuccinate taken p.o. in a capsular formulation, for example, is indicated in those with a positive test, with the aim of lessening the rate of viral replication and hence the severity of any attendant symptoms or signs, including those pertaining to long COVID. As noted, Example 1 using a radio-labeled triethylenetetramine disuccinate provides direct evidence for those organs that the drug accesses after oral administration in a relevant animal model.

When not given prophylactically, triethylenetetramine disuccinate, for example, taken p.o. in a capsular formulation (by way of one example of administration) is indicated for the treatment of those patients with asymptomatic or symptomatic viral disease either in the community or in hospital or other care settings in whom there is evidence of on-going viral replication, for example as indicated by one or more positive tests for the virus. Triethylenetetramine disuccinate, for example, may be taken 3×500 or 522 mg caps BID until 21 days after the last positive test for the virus. See Examples 13 and 14. Other doses are also appropriate, as described herein. If necessary, retesting should be performed until (or, if desired, beyond) a 21-day period has elapsed since the last positive test for the virus, or any other period as fits current thinking for coronaviruses. If viral positivity re-emerges after one or more negative tests, then a second course of a copper-depriving compound, triethylenetetramine disuccinate, for example, is indicated until it meets the criterion of 21-days has elapsed after the last positive test, or another desired period based on coronavirus knowledge.

Triethylenetetramine disuccinate, for example, taken p.o. at 3×500 or 522 mg caps BID, again by way of example in a capsular or other formulation should be taken prophylactically, as a preventative treatment, in those patients are shown to have been contacts of people who are known or thought to have been exposed to the virus to minimize the risk of establishment of the infectious syndrome.

Administration of copper-depriving compounds, including triethylenetetramine disuccinate, for example, is also indicated for patients being treated in the hospital for severe symptomatic disease of the upper respiratory tract or the lungs or any of the other organs that may be affected in long forms of the viral disease including but not limited to the brain, heart, kidneys, gut, immune system, or other organ systems, preferably those whose treatment does not involve general anesthesia and ventilation, as well as treatment of other post-viral syndromes.

Administration of copper-depriving compounds, including triethylenetetramine disuccinate, for example, via gastric lavage in water or other accepted aqueous solutions such as physiological saline for intra-gastric administration is indicated in those patients whose treatment involves general anesthesia and ventilation in a hospital setting at the equivalent of 3×522 mg of TES from suitably-labeled capsules is administered BID, or other doses as described herein. To enable this mode of treatment, for example, powder from triethylenetetramine disuccinate capsules or other forms of copper-depriving compounds, may be dissolved in water or other appropriate aqueous media, for example physiological saline, as is known to those skilled in the art.

Host cell copper transporters CTR1 and ATP7A are essential for host cells, and triethylenetetramine interacts with CTR1 and ATP7A in a manner consistent with a therapeutic effect on coronaviruses. Triethylenetetramine, in particular triethylenetetramine disuccinate, is a preferred copper chelator for the prevention and treatment of coronavirus infection because of its clean safety profile.

In another aspect, the fixed dose of triethylenetetramine disuccinate is used in combination with an inhibitor of N-acetylaminotransferase. In another aspect, the fixed dose of triethylenetetramine disuccinate is used in combination with an inhibitor of spermidine-spermine-N(1)-acetyltransferase (SSAT1 and/or SSAT2). In one preferred embodiment, the fixed dose of triethylenetetramine disuccinate is used in combination with an inhibitor of spermidine-spermine-N(1)-acetyltransferase-2 (SSAT2).

In another aspect, the article of manufacture comprises a number of capsules equal to a daily dose of triethylenetetramine disuccinate, wherein the daily dose is selected from the group consisting of from about 2400 mg per day to about 3000 mg per day of triethylenetetramine disuccinate.

In another aspect, the triethylenetetramine disuccinate in the article of manufacture of has a purity of at least about 95%. In a further aspect, the purity is at least about 99%.

In another aspect, the triethylenetetramine disuccinate in the article of manufacture is a triethylenetetramine disuccinate anhydrate.

In another aspect, the triethylenetetramine disuccinate in the article of manufacture is non-hygroscopic and possesses good stability under conditions of normal, room temperature storage. Importantly, the crystalline anhydrous form of the triethylenetetramine disuccinate article of manufacture described herein has a shelf-life of at least about 12 months (and up to five years) at room temperature, without significant degradation of the triethylenetetramine disuccinate API and remains within impurity specifications for the triethylenetetramine disuccinate drug substance. In one embodiment, the term “without significant degradation” means that the purity of the triethylenetetramine disuccinate is at least about 98.5% with no degradation product above about 0.5% and no new, unidentified impurities above about 0.1% for at least about 12 months.

In another aspect, the article of manufacture with a fixed dose of triethylenetetramine disuccinate is in the form of a capsule. In another aspect, the article of manufacture with a fixed dose of triethylenetetramine disuccinate is in the form of a tablet. In a further aspect, the capsule or tablet of triethylenetetramine disuccinate is formulated in a manner so as to provide delayed or sustained release, thereby resulting in a modified pharmacokinetic profile from a related immediate-release form.

In a still further aspect, the invention also comprises a method of managing or treating a subject with a disease treatable with a copper chelator, the method comprising administering triethylenetetramine disuccinate to said subject in an amount ranging from about 2400 mg per day to about 3000 mg per day of triethylenetetramine disuccinate. In one aspect of the method, the disease treatable with a copper chelator is characterized by excess copper. In another aspect, the triethylenetetramine disuccinate used in the methods is at least about 95% pure, at least about 99% pure, or 100% pure. In another aspect, the triethylenetetramine disuccinate used in the method is a crystalline form of triethylenetetramine disuccinate. In yet another aspect of this method, the triethylenetetramine disuccinate is a triethylenetetramine disuccinate anhydrate. In still another aspect of the method the triethylenetetramine disuccinate is in the form of a fixed dose tablet or capsule. In one preferred embodiment, the fixed dose of triethylenetetramine disuccinate is about 400 mg, about 500 mg, about 600 mg or about 700 mg. In another preferred embodiment of the method the subject is a human.

In another embodiment, three fixed dose tablets or capsules of the 400 mg fixed dose of triethylenetetramine disuccinate is given twice per day (2400 mg per day).

In another embodiment, three fixed dose tablets or capsules of the 500 mg fixed dose of triethylenetetramine disuccinate is given twice per day (3000 mg per day).

In another embodiment, the triethylenetetramine disuccinate fixed dose tablets or capsules are 350 mg.

In another embodiment, the total amount given per day is 2800 mg as four 350 mg tablets or capsules BID.

In another aspect, the fixed dose of triethylenetetramine disuccinate is used to lower or normalize copper(II) content in a subject. In one embodiment, the fixed dose of triethylenetetramine disuccinate reduces total copper in the subject. In another embodiment, the fixed dose of triethylenetetramine disuccinate is used to treat a subject for a disease, disorder or condition who would benefit from a copper(II) chelator.

In one preferred embodiment of methods of the invention, fixed dose of triethylenetetramine disuccinate is delivered orally.

In another embodiment, a fixed dose of triethylenetetramine disuccinate maintains total copper in the subject within the normal human serum or plasma range of about 0.8-1.2 milligrams/L, or about 10-25 micromoles/L. In another embodiment, the fixed dose of triethylenetetramine disuccinate maintains total copper in the subject within at least about 70% of the normal range of about 0.8-1.2 milligrams/L or about 10-25 micromoles/L, e.g., at least about 75%. In another embodiment, fixed dose of triethylenetetramine disuccinate maintains total copper in the subject within about 75% to about 85%, or about 85% to about 95% the normal range of copper in human plasma or serum. In one aspect of the methods of the invention, the copper status of a subject given a fixed dose of triethylenetetramine disuccinate is determined by evaluating copper in the urine of the subject.

In one aspect of the invention, the method employs a pharmaceutical composition comprising a fixed dose of substantially pure triethylenetetramine disuccinate. In another aspect the method employs a pharmaceutical composition comprising substantially pure triethylenetetramine disuccinate and a pharmaceutically acceptable excipient.

In one aspect of the invention, the method employs a fixed dose of a crystalline form of triethylenetetramine disuccinate.

In another aspect of the invention, the method employs a fixed dose of triethylenetetramine disuccinate anhydrate.

In certain embodiments, the fixed dose of triethylenetetramine succinate is a triethylenetetramine disuccinate polymorph.

A preferred pharmaceutical composition for use in the methods of the invention comprises or consists essentially of or consists of a fixed dose of substantially pure triethylenetetramine disuccinate. Another preferred composition is a fixed dose of substantially pure triethylenetetramine disuccinate anhydrate. Another preferred composition is a composition that comprises or consists essentially of or consists of a fixed dose of a substantially pure triethylenetetramine disuccinate crystal having alternating layers of triethylenetetramine molecules and succinate molecules.

In another aspect of the invention, the method maintains copper levels with about 70% to about 100% of normal in the subject, thereby eliciting by a lowering of copper values in a mammalian patient and/or reducing the level of copper.

The total dosage of triethylenetetramine disuccinate may be given in single or divided dosage units (e.g., BID, TID), and preferably maintain normal urine and/or plasma copper levels in a subject, or levels that do not fall below about 70% to 75% of normal. Fixed doses of triethylenetetramine disuccinate are typically administered BID.

In some embodiments, the method comprises or consists essentially of or consists of administering a tablet or capsule comprising a fixed dose of triethylenetetramine disuccinate to a subject. Preferably, the fixed dose of triethylenetetramine disuccinate is administered orally in the form of a capsule.

In any of the procedures described and/or claimed herein, the fixed triethylenetetramine disuccinate dosage regimen given to a subject will not reduce physiological levels of copper down to a depletion state or to an otherwise dangerously low level in the subject.

The invention also includes an article of manufacture, e.g., a kit of parts, comprising or consisting essentially of one or more of the fixed doses of triethylenetetramine disuccinate described herein, for example, oral fixed doses of triethylenetetramine disuccinate, and a printed set of instructions (e.g., a package insert) describing their use in therapy, for example in the treatment of heart failure, diabetic cardiomyopathy, left ventricular hypertrophy, Wilson's disease, cancer, etc. In one embodiment, the kit does not include a physical set of instructions, but refers to or describes their availability online, in the cloud, in a flash drive, or another storage mechanism. In one embodiment, the instructions recite that the triethylenetetramine disuccinate is to be administered to patients with Wilson's disease previously receiving triethylenetetramine dihydrochloride or DPA.

These discoveries provide a molecular mechanism of action for the treatment of coronavirus infection by copper chelator treatment, including the coronaviruses that lead to COVID-19 disease. These drugs may well also be efficacious in treating coronaviruses-evoked damage in organ systems other than the respiratory system and hear, including for example, the brain, kidneys, blood, bone marrow, and immune system, and also severe post-viral syndromes including late-onset manifestations of organ damage and post-viral fatigue syndromes. Members of other viral families, including single-stranded RNA viruses and share structural and functional similarities with coronaviruses, may also be susceptible to this treatment. Effective doses are described.

The invention includes a method of treating coronavirus disease in a subject caused by exposure to a copper-requiring coronavirus, the method comprising administering to the subject a composition comprising an effective amount of a copper-depriving agent, wherein one or more symptoms of the disease are reduced. In some embodiments, the coronavirus infection is caused by a SARS-CoV-2 coronavirus. In some embodiments, the coronavirus disease is COVID-19 disease. In some embodiments, the copper depriving agent lowers copper values content and/or reduces intracellular copper in the subject. In some embodiments, the copper depriving agent reduces total copper. In some embodiments, the copper depriving agent is a copper chelating compound. In some embodiments, the copper chelating compound preferentially binds Cu¹⁺. In some embodiments, the copper chelating compound preferentially binds Cu²⁺. In some embodiments, the copper chelating compound binds both Cu¹⁺ and Cu²⁺. In some embodiments, the copper depriving agent reduces intracellular copper. In some embodiments, the copper depriving agent inhibits copper transport into a copper-requiring coronavirus host cell. In some embodiments, the copper-requiring coronavirus is a SARS coronavirus (a SARS-CoV), a MERS coronavirus (a MERS-CoV), a COVID-19 coronavirus (a SARS-CoV-2), a human 229E coronavirus, a human OC43 coronavirus, a human HCoV-NL63 coronavirus or a human HKU1 coronavirus. In some embodiments, the copper-requiring coronavirus is an infectious SARS-CoV-2 virus. In some embodiments, the chelator is selected from the group consisting of triethylenetetramine, ammonium tetrathiomolybdate, D-penicillamine and N-acetylpenicillamine. In some embodiments, the copper-depriving compound is a triethylenetetramine. In some embodiments, the triethylenetetramine is a hydrochloric acid salt of triethylenetetramine. In some embodiments, the triethylenetetramine hydrochloric acid salt is triethylenetetramine dihydrochloride or triethylenetetramine tetrahydrochloride. In some embodiments, the triethylenetetramine is a succinic acid salt of triethylenetetramine. In some embodiments, the triethylenetetramine succinic acid salt is triethylenetetramine disuccinate. In some embodiments, the triethylenetetramine disuccinate is substantially pure. In some embodiments, the triethylenetetramine disuccinate is a crystalline form of triethylenetetramine disuccinate. In some embodiments, the triethylenetetramine disuccinate is triethylenetetramine disuccinate anhydrate. In some embodiments, the triethylenetetramine disuccinate is a triethylenetetramine disuccinate polymorph. In some embodiments, the composition comprising triethylenetetramine is formulated in a capsule for oral administration. In some embodiments, the composition is formulated for nasal, intrasinal, intrapulmonary and/or endosinusial administration. In some embodiments, the copper-depriving agent is a copper chelator and is administered in an amount ranging from about 600 to about 2400 milligrams per day. In some embodiments, the copper chelator is administered in an amount ranging from about 1200 to about 2400 milligrams per day. In some embodiments, the about 1200 milligrams per day of the agent effective to lower the copper values is administered in separate doses each equal to about 600 mg.

The invention also provides a method of preventing or treating coronavirus infection in a subject caused by exposure to a copper-requiring coronavirus, the method comprising administering to the subject, either before or after the exposure, a composition comprising an effective amount of a copper-depriving agent that lowers copper available to a coronavirus values by removing copper from or reducing intracellular copper in the subject, wherein the method results in reducing infectious coronavirus organisms and/or coronavirus particles and preventing infection or reducing the infection in the subject. The invention also provides a method of preventing or reducing coronavirus infection in a subject caused by exposure to a COVID-19 coronavirus, the method comprising administering to the subject, either before or after the exposure, a composition comprising an effective amount of a copper-depriving compound selected from the group consisting of triethylenetetramine, ammonium tetrathiomolybdate, D-penicillamine and N-acetylpenicillamine, wherein the method results in reducing infectious coronavirus organisms and/or coronavirus particles and preventing infection or reducing the infection in the subject. In some embodiments, the administration provides a prophylactic effect against viral infection for at least about 12 to 24 hours. In some embodiments, the administration provides a prophylactic effect for at least about 24 to 48 hours. In some embodiments, the administration provides a prophylactic effect for at least about 48 to 72 hours. In some embodiments, the administration: (a) increases the chance of survival following exposure to a copper-requiring coronavirus; and/or (b) reduces the colonization of a copper-requiring coronavirus in the nose or in the lung; and/or (c) reduces the risk of transmission of the copper-requiring coronavirus. In some embodiments, the survival of the subject is increased. In some embodiments: (a) the coronavirus comprises human coronavirus 229E, human coronavirus OC43, SARS-CoV, a HCoV-NL63, HKU1, MERS-CoV, or SARS-CoV-2; and/or (b) the risk of infection to be prevented or reduced is by coronavirus disease 2019 (COVID-19); and/or (c) the coronavirus comprises (i) a polynucleotide comprising SARS-CoV-2 (GenBank accession number NC_0455122), or (ii) a copper-requiring strain or mutation thereof, or (iii) an infectious fragment thereof coding for or included within a viable or infectious viral particle susceptible to copper deprivation, or (iv) a copper-requiring infectious polynucleotide having at least 80% sequence identity to the polynucleotide comprising SARS-CoV-2.

In some embodiments, the administering comprises administration of a nasal spray, medicated nasal swab, medicated wipe or aerosol comprising the composition to the subject's nasal vestibule or nasal passages.

In some embodiments, the subject is exposed to or is anticipated to be exposed to an individual with one or more symptoms selected from the group consisting of fever, cough, shortness of breath, diarrhea, sneezing, runny nose, and sore throat.

In some embodiments, the subject is a healthcare worker, elderly person, frequent traveler, military personnel, caregiver, within the BAME group, or a subject with a preexisting condition that results in increased risk of mortality with infection, and optionally wherein the preexisting condition comprises cancer, chronic kidney disease, chronic obstructive pulmonary disease, organ transplant, sickle cell disease, diabetes, type 2 diabetes, type 1 diabetes, hypertension, obesity, pulmonary fibrosis, heart disease or an immunocompromised state.

In some embodiments, the administering further comprises administration of one or more antiviral drugs. In some embodiments, the one or more antiviral drugs is/are selected from the group consisting of chloroquine, hydroxychloroquine, darunavir, galidesivir, an interferon, lopinavir, ritonavir, remdesivir, and triazavirin. In some embodiments, the interferon is selected from the group consisting of interferon β-1b, pegylated interferon β-1b, interferon α-n1, pegylated interferon α-n1, interferon α-n3, pegylated interferon α-n3 and human leukocyte interferon α.

In some embodiments, the composition comprising an effective amount of a copper-depriving agent further comprises a therapeutic agent, and optionally wherein the therapeutic agent is: (a) an antimicrobial agent; an antiviral agent; an antifungal agent; vitamin; homeopathic agent; anti-inflammatory agent; keratolytic agent; antipruritic agent; pain medicine; steroid; naloxone; and a combination thereof; and/or (b) selected from the group consisting of a penicillin, a cephalosporin, cycloserine, vancomycin, bacitracin, miconazole, ketoconazole, clotrimazole, polymyxin, colistimethate, nystatin, amphotericin B, chloramphenicol, a tetracycline, erythromycin, clindamycin, an aminoglycoside, a rifamycin, a quinolone, trimethoprim, a sulfonamide, zidovudine, gangcyclovir, vidarabine, acyclovir, poly(hexamethylene biguanide), terbinafine, and a combination thereof; (c) an anti-inflammatory agent; and/or (n) an anti-inflammatory agent which is a steroid or a non-steroidal anti-inflammatory drug; and/or (o) an anti-inflammatory agent which is a steroid selected from the group consisting of clobetasol, halobetasol, halcinonide, amcinonide, betamethasone, desoximetasone, diflucortolone, fluocinolone, fluocinonide, mometasone, clobetasone, desonide, hydrocortisone, prednicarbate, triamcinolone, and a pharmaceutically acceptable derivative thereof; and/or (p) an anti-inflammatory agent which is a non-steroidal anti-inflammatory drug selected from the group consisting of aceclofenac, aspirin, celecoxib, clonixin, dexibup6fen, dexketoprofen, diclofenac, diflunisal, droxicam, etodolac, etoricoxib, fenoprofen, flufenamic acid, flurbiprofen, ibuprofen, indomethacin, isoxicam, ketoprofen, ketorolac, licofelone, lornoxicam, loxoprofen, lumiracoxib, meclofenamic acid, mefenamic acid, meloxicam, nabumetone, naproxen, nimesulide, oxaprozin, parecoxib, phenylbutazone, piroxicam, rofecoxib, salsalate, sulindac, tenoxicam, tolfenamic acid, tolmetin, or valdecoxib.

In some embodiments, administration of the composition comprising an effective amount of a copper-depriving agent is once, twice, three times, or more than three times per day.

The invention also provides articles of manufacture for use in treating coronavirus disease comprising a single dose capsule or tablet containing a single fixed dose of triethylenetetramine disuccinate, wherein the fixed dose is selected from the group consisting of about 350 mg, about 584 mg and about 701 mg of triethylenetetramine disuccinate. In some embodiments, the article of manufacture comprising a number of fixed dose capsules equal to one or more daily doses of triethylenetetramine disuccinate, wherein the daily dose is selected from the group consisting of from about 1050 mg per day to about 2300 mg per day, about 1400 mg per day to about 3500 mg per day, about 2300 mg per day to about 2800 mg per day, and about 2800 mg per day to about 5600 mg per day of triethylenetetramine disuccinate and, optionally, wherein the wherein the fixed dose is selected from the group consisting of about 350 mg, about 400 mg, about 500 mg, about 584 mg about 600 mg and about 701 mg of triethylenetetramine disuccinate.

In some embodiments, the triethylenetetramine disuccinate in the article of manufacture is a crystalline form of triethylenetetramine disuccinate. In some embodiments, the triethylenetetramine disuccinate in the article of manufacture is a triethylenetetramine disuccinate anhydrate. In some embodiments of the article of manufacture, the fixed dose of triethylenetetramine disuccinate is about 600 mg. In some embodiments of the article of manufacture, the fixed dose of triethylenetetramine disuccinate is about 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg or 3600 mg.

In some embodiments of the article of manufacture, the fixed dose of triethylenetetramine disuccinate is in the form of a capsule or tablet. In some embodiments, the capsule or tablet is formulated to provide for a delayed release. In some embodiments, the capsule or tablet is formulated to provide for a sustained release. In some embodiments of the article of manufacture, the capsule or tablet is formulated in combination with a pharmacokinetic enhancer (PKE) that provides for improved absorption of the triethylenetetramine disuccinate.

The invention also provides for an article of manufacture comprising triethylenetetramine disuccinate and an inhibitor of N-acetylaminotransferase. In some embodiments, the inhibitor of N-acetylaminotransferase is an inhibitor of spermine/spermidine N-acetyltransferase (SSAT1). In some embodiments, the inhibitor of N-acetylaminotransferase is an inhibitor of spermine/spermidine N-acetyltransferase (SSAT2).

The invention also provides for an article of manufacture article of manufacture comprising triethylenetetramine disuccinate and a promoter of polyamine membrane transport including bergamottin, maringenin, quercetin, other psoralens, piperine, or tetrahydro-piperine that act as enhancers of membrane permeability for increased absorption.

In some embodiments, the fixed dose triethylenetetramine disuccinate capsule or tablet has a shelf-life term of at least about 12 months at room temperature. In some embodiments, the minimum purity of the triethylenetetramine disuccinate over said shelf-life term is least about 98.5% with no degradation product above about 0.5% and no new, unidentified impurities above about 0.1%. In some embodiments, the shelf-life term of the article of manufacture is about 12 months.

### Definitions

Copper(I) and copper(II) referred to herein are also known as copper⁺¹ and copper⁺², respectively, or as “cuprous” (the copper⁺¹ cation) and “cupric” (the copper⁺² cation), or as Cu⁺¹ and Cu⁺², respectively.

The term “comprising,” which is synonymous with “including,” “containing,” or “characterized by,” is inclusive or open-ended and does not exclude additional, unrecited elements or ingredients from the medicament (or steps, in the case of a method). The phrase “consisting of” excludes any element, step, or ingredient not specified in the medicament (or steps, in the case of a method). The phrase “consisting essentially of” refers to the specified materials and those that do not materially affect the basic and novel characteristics of the medicament (or steps, in the case of a method). The basic and novel characteristics of the inventions are described throughout the specification, and include the ability of compounds, compositions and methods of the invention to deprive a coronavirus of copper, and/or to block or modulate the lifecycle of a copper-requiring coronavirus, and/or to provide a clinically relevant change in a coronavirus disease state, symptom or infection, e.g., a COVID-19 disease state, symptom or infection. The basic and novel characteristics of other compositions and methods of the invention include the ability to reduce inflammation or combat viruses.

As used herein the terms “subjecting the patient” or “administering to” includes any active or passive mode of ensuring the in vivo presence of the active compound(s) or metabolite(s) irrespective of whether one or more dosage to the mammal, patient or person is involved. Preferably the mode of administration is nasal or oral. However, all other modes of administration (particularly parenteral, e.g., intravenous, intramuscular, etc.) are also contemplated.

As used herein, the term “subject” or the like, including “individual,” and “patient”, all of which may be used interchangeably herein, refers to any mammal, including humans. The preferred mammal herein is a human, including adults, children, including those with Wilson's Disease, heart failure, cardiomyopathy, diabetes or cancer, by way of example. In certain embodiments, the subject, individual or patient is a human. In some respects, the patient is in a group with a higher mortality rate risk from, e.g., COVID-19, such as BAME patients (Black, Asian and Minority Ethnic groups), elderly persons over 60-65, 70, 75, 80, 85, 90 or 95 years of age, etc. Other high-risk subjects include those described herein, including people with cancer, chronic kidney disease, chronic obstructive pulmonary disease, heart conditions, organ transplant, sickle cell disease, etc., and Type 2 diabetes, as well as people with Type 1 diabetes, those with immunocompromised state and those with pulmonary fibrosis.

As used herein, “mammal” has its usual meaning and includes primates (e.g., humans and nonhumans primates), experimental animals (e.g., rodents such as mice and rats), farm animals (such as cows, hogs, minks (who are serious coronavirus carriers), chickens, ducks, sheep and horses), and domestic animals (such as dogs and cats). The invention relates to the treatment of any mammal that is infected by, or at risk from being infected by, a copper-requiring or copper-dependent coronavirus. Humans are a preferred treatment subject. In one aspect of the invention, a copper-depriving agent is added to animal feed or water, and the invention includes animal feed or water with one or more copper-depriving agents.

The term “treating coronavirus disease” or the like, refers to preventing, slowing, reducing, decreasing, stopping and/or reversing coronavirus disease or infection, such as, for example, one or more symptoms thereof.

The term “treating coronavirus disease” or the like, also refers to preventing, slowing, reducing, decreasing, stopping and/or reversing long COVID. “Long Covid” (also “long-haul Covid”, “Chronic Covid”, “Chronic Covid Syndrome”, “CCS”) describes long-term sequelae of coronavirus disease 2019 (COVID-19) in which about 10 to 20 percent of people who have been diagnosed with COVID-19 report experiencing a range of symptoms lasting longer than a month, and 2.3 percent (1 in 44 people) report having symptoms which last longer than 12 weeks. These long-term symptoms include extreme fatigue, headache, dyspnoea (dyspnea, commonly referred to as simply “shortness of breath”), anosmia, muscle weakness, low grade fever, cognitive dysfunction (“brain fog”), hair loss and teeth loss.

“Treating coronavirus infection,” including SARS-CoV-2 infection, refers to preventing, slowing, reducing, decreasing, stopping and/or reversing the infection, including, for example, one or more symptoms thereof, including those described herein.

The term “preventing” means preventing in whole or in part or ameliorating or controlling. Thus, preventing a disease means preventing in whole or in part, or ameliorating or controlling the disease, e.g., COVID-19 disease.

As used herein, the terms “effective amount” or “therapeutically effective amount” refer to a sufficient amount of the agent to provide the desired biological result. That result can be reduction and/or alleviation of the signs, symptoms, or causes of coronavirus disease or infection, e.g., COVID-19 disease or infection. For example, an “effective amount” for therapeutic use is the amount of a compound that deprives a coronavirus of copper, or of a composition comprising that compound, that is useful or required to provide a clinically relevant change in a coronavirus disease state, symptom or infection, e.g., a COVID-19 disease state, symptom or infection. An appropriate “effective” amount in any individual case may be determined by those in the art using the information provided herein. Thus, the expression “effective amount” generally refers to the quantity for which the active substance has a therapeutically desired effect. Effective amounts or doses of the compounds of the embodiments may be ascertained by various methods, such as modeling, dose escalation, or clinical trials, taking into account various factors, e.g., the mode or route of administration or drug delivery, the pharmacokinetics of the copper-depriving agent or composition, the severity and course of the infection, the subject's health status, condition, and weight, and the judgment of the treating physician. Some exemplary effective amounts are described herein, and include, by way of example, doses in the range of about 1 to 20 mg of active agent per kilogram of subject's body weight per day, preferably about 7 to about 18 mg/kg/day, or about 8 to 17 mg/kg/day, or about 10 to 15 mg/kg/day. Other doses include doses up to about 34 mg/kg of a copper-depriving agent or composition. Other doses are provided elsewhere herein. In one embodiment, a copper-depriving compound may be administered at dosages or a dosage to provide, if parenteral, at least about 120 mg/day in a human patient, and if oral, at least about 1200 mg/day in a human patient. Some oral doses of copper-depriving compounds may be administered at about 600 to about 1200 to about 2400 mg/day. The total dosage may be given in single or divided dosage units (e.g., BID, TID or QID), and preferably maintain normal urine and/or plasma copper levels in a subject, or levels that do not fall below about 70% to 75% of normal. BID is currently preferred.

Thus, in one aspect, “effective amount” refers to an amount effective, at dosages and for periods of time necessary, to achieve the desired therapeutic or prophylactic result. For example, and not by way of limitation, an “effective amount” can refer to an amount of a copper-depriving compound or composition, including but not limited to those disclosed herein, that is able to treat the signs and/or symptoms of the coronavirus disease, disorder or condition, e.g., COVID-19 disease. In one embodiment, the effectiveness of the amount is evaluated by determining the response of the target virus and/or the amount copper in the urine or plasma in a subject to a copper-depriving compound or composition. Preferably, as noted, the effective amount maintains normal copper levels while interrupting the target coronavirus activity, and maintains a subject's copper levels within at least about 70% of normal, or within other levels described herein. And another aspect, “therapeutically effective amount” of a virus copper-depriving compound of the invention, may vary according to factors such as the disease state, age, sex, and weight of the individual, of course, and the ability of the copper-depriving compound to elicit a desired response in the individual. A therapeutically effective amount is preferably also one in which any toxic or detrimental effects of the copper-depriving compound may be outweighed by the therapeutically beneficial effects. A “therapeutically effective amount” is typically a predetermined amount of an agent that will or is calculated to achieve a desired response (see “effective amount”), for example, a therapeutic or preventative or ameliorating response, for example, a biological or medical response of a tissue, system, animal or human that is sought, for example, by a researcher, veterinarian, medical doctor, or other clinician.

As used herein, “prophylactically effective amount” refers to an amount effective, at dosages and for periods of time necessary, to achieve a desired prophylactic result. Typically, but not necessarily, since a prophylactic dose is used in subjects prior to or at an earlier stage of a coronavirus disease, infection or symptomology, the prophylactically effective amount may be less than the therapeutically effective amount.

Patients may be given prophylactically effective amounts in accordance with methods of the invention. Copper-depriving agents may also be used, for example, as a prophylactic treatment combined with (or in conjunction with the administration of) coronavirus vaccines, such as one or more of the vaccines for the COVID-19 virus, and/or variants thereof.

The invention also includes the use of copper-sequestering or copper-depriving agents to improve vaccine efficacy. In one embodiment, for example, the copper-depriving agent triethylenetetramine disuccinate is administered with or in conjunction with a coronavirus vaccine, e.g., a COVID-19 virus vaccine. This method is particularly valuable for high-risk groups.

In another aspect, the invention includes a composition of matter comprising or consisting essentially of a copper-depriving agent and coronavirus vaccine. In one embodiment, for example, the composition comprises or consists essentially of triethylenetetramine disuccinate and a COVID-19 virus vaccine. This composition is administered to high-risk groups.

By “pharmaceutically acceptable” it is meant, for example, a carrier, diluent or excipient that is compatible with the other ingredients of the formulation and generally safe for administration to a recipient thereof or that does not cause an undesired adverse physical reaction upon administration.

As used herein, the terms “treatment” or “treating” of the signs and/or symptoms of a coronavirus disease, disorder or condition, e.g., COVID-19 disease, disorder, and/or condition in a mammal, means, where the context allows, (i) preventing the condition or disease, that is, avoiding one or more clinical symptoms of the disease; (ii) inhibiting the condition or disease, that is, arresting the development or progression of one or more clinical symptoms, or the virus itself; and/or (iii) relieving the condition or disease, that is, causing the regression of one or more clinical symptoms. Thus, “treatment” (and grammatical variations thereof such as “treat” or “treating”) normally refers to clinical intervention in an attempt to alter the natural course of the individual, tissue or cell being treated, and can be performed either for prophylaxis or during the course of clinical pathology. Desirable effects of treatment include, but are not limited to, preventing occurrence or recurrence of a coronavirus disease, disorder or condition, or infection, alleviation of signs or symptoms, diminishment of any direct or indirect pathological consequences of the coronavirus disease, decreasing the rate of coronavirus disease progression, amelioration or palliation of the coronavirus disease state, and remission or improved prognosis. In some embodiments, the copper-depriving compounds, methods and compositions of the invention can be used to delay development of a coronavirus disease, disorder or condition, or infection, or to slow the progression of a coronavirus disease, disorder or condition, or infection. The term does not necessarily imply that a subject is treated until total recovery. Accordingly, “treatment” includes reducing, alleviating or ameliorating the symptoms or severity of a coronavirus disease, disorder or condition, or infection, or preventing or otherwise reducing the risk of developing a coronavirus disease, disorder or condition, or infection. It may also include maintaining or promoting a complete or partial state of remission of a coronavirus condition or infection.

As used herein “associated with” simply means both circumstances exist and should not be interpreted as meaning one necessarily is causally linked to the other.

The term “chelatable copper” includes copper in any of its chelatable forms including different oxidation states such as copper(I) and copper(II). Accordingly, the term “copper values” (for example, elemental, salts, etc.) means copper in any appropriate form in the body available for such chelation (for example, in extracellular tissue and possibly bound to cell exteriors and/or collagen as opposed to intracellular tissue) and/or capable of being reduced by other means. Certain methods and compositions of the invention may be used to bind chelatable copper, for example, chelatable copper (II) to deprive a coronavirus of copper while maintaining normal or near-normal copper values (e.g., within about 70-75% of normal, for example, or other copper values amount not detrimental to the subject).

The term “pharmaceutical formulation” refers to a preparation which is in such form as to permit the biological activity of an active ingredient contained therein to be effective, and which does not contain additional components that are unacceptably toxic to a subject to which the formulation would be administered. Pharmaceutical formulations of the invention comprise a copper-depriving agent, e.g., a copper chelator (alone or together with an antiviral and/or anti-inflammatory agent).

“Copper chelating agents” bind or modify copper, including those that selectively bind to or modify copper(I) or copper (II) values and are used to normalize blood and/or tissue copper levels and to prevent unwanted copper accumulation. Copper chelating agents include prodrugs thereof. Other agents that normalize copper values, and other agents that selectively bind to or modify copper (II), whether now known or later developed, are included within this definition.

A “copper sequestering agent” or “copper-depriving agent” is an agent that can bind to and/or suppress the ability of copper in any or all of its various forms, for example, as copper atoms or copper ions, to interact in any chemical or physical reactions that it could otherwise do, including a copper-dependent process in an organism such as a microbe, bacterium, or virus, including an RNA virus. Copper-depriving agents include chelators, agents that reduce total copper, agents that reduce copper values, agents that reduce the amount of intracellular copper available to a coronavirus, etc., including those described herein. Copper-depriving agents also include copper-modifying agents, i.e., agents used to deprive a virus of copper by modifying copper content in the body, including intracellular content, or by modifying copper availability. It is understood that copper is an essential intracellular nutrient, and thus the invention includes methods to reduce intracellular copper content while maintaining safe patient copper levels. Copper-depriving agents include copper-removing agents, i.e., agents that remove copper from the body and/or from inside cells.

As used herein, the term “subject” or the like, including “individual,” and “patient”, all of which may be used interchangeably herein, refers to any mammal susceptible to a coronavirus, e.g., a SARS-CoV-2 coronavirus, including humans. The preferred mammal herein is a human, including adults, children, and the particularly the elderly. In certain embodiments, the subject, individual or patient is a human.

Copper chelating agents, copper sequestering agents, copper depriving agents, alone or together with other agents, including antivirals and anti-inflammatories, may be administered alone or in combination with one or more additional ingredients and may be formulated into pharmaceutical compositions including one or more pharmaceutically acceptable excipients, diluents and/or carriers.

A “pharmaceutically acceptable carrier,” as used herein, refers to an ingredient in a pharmaceutical formulation, other than an active ingredient, which can be safely administered to a subject. A pharmaceutically acceptable carrier includes, but is not limited to, a buffer, excipient, stabilizer, or preservative. Pharmaceutically acceptable diluents, carriers and/or excipients include substances that are useful in preparing a pharmaceutical composition, may be co-administered with compounds described herein while allowing them to perform its intended functions, and are generally safe, non-toxic and neither biologically nor otherwise undesirable. Pharmaceutically acceptable diluents, carriers and/or excipients include those suitable for veterinary use as well as human pharmaceutical use. Suitable carriers and/or excipients will be readily appreciated by persons of ordinary skill in the art, having regard to the nature of compounds of the invention. However, by way of example, diluents, carriers and/or excipients include solutions, solvents, dispersion media, delay agents, polymeric and lipidic agents, microspheres, emulsions and the like. By way of further example, suitable liquid carriers, especially for injectable solutions, include water, aqueous saline solution, aqueous dextrose solution, and the like, with isotonic solutions being preferred for intravenous, intraspinal, and intracisternal administration and vehicles such as liposomes being also suitable for administration of the agents of the invention.

In certain embodiments, the invention provides a combination product comprising (a) a copper chelating agent(s), copper sequestering agent(s), copper depriving agent(s), for example a copper (II) chelator (e.g., a succinic acid addition salt of triethylenetetramine, such as triethylenetetramine disuccinate), and (b) one or more anti-inflammatory agents and/or other anti-viral agents, wherein the components (a) and (b) are adapted for administration simultaneously or sequentially. In a particular embodiment of the invention, a combination product in accordance with the invention is used in a manner such that at least one of the components is administered while the other component is still having an effect on the subject being treated.

The copper chelating agent(s), copper sequestering agent(s), copper depriving agent(s) and/or anti-inflammatory agents and/or other anti-viral agents may be contained in the same or one or more different containers and administered separately, or mixed together, in any combination, and administered concurrently. Preferably, both or all three of the copper depriving agent and/or anti-inflammatory agent and/or anti-viral agent are combined in a capsule for oral administration.

Such combination products may be manufactured in accordance with the methods and principles provided herein and those known in the art. Also provided is combination product used in a method as herein described.

For separate or common administration, the formulation may be prepared to provide for rapid or slow release; immediate, delayed, timed, or sustained release; or a combination thereof. Formulations may be in the form of liquids, solutions, suspensions, emulsions, elixirs, syrups, electuaries, drops (including but not limited to eye drops), tablets, granules, powders, lozenges, pastilles, capsules, gels, ointments, creams, lotions, oils, foams, sprays, mists, or aerosols. As an additional embodiment, the pharmaceutical formulation can be contained within, delivered by, or attached to a swab that is used to administer drug, for example, in the nose.

It is understood that the lungs are the organs most affected by COVID-19 because the virus accesses host cells via the enzyme angiotensin-converting enzyme 2 (ACE2), which is most abundant in the type II alveolar cells of the lungs. The virus can not only damage the lung, but also the heart, liver and kidney, which can explain some of the severe COVID-19 complications in people. Reviewed in Dong, M., et al. ACE2, TMPRSS2 distribution and extrapulmonary organ injury in patients with COVID-19 Biomedicine & Pharmacotherapy, Vol. 131, November 2020, 110678, where the authors describe that COVID-19 in a proportion of patients is accompanied by extrapulmonary symptoms including cardiac injury, kidney injury, liver injury, digestive tract injury, and neurological symptoms.

Copper depriving agents, including triethylenetetramine disuccinate, are not only taken up into the lungs, but into the kidney and liver, amongst many other tissues, including the nasal mucosa, where SARS-CoV-2 entry factors are highly expressed in nasal epithelial cells. See Example 1.

### Copper Depriving Agents

Agents that sequester, bind or chelate copper and/or otherwise deprive a copper-requiring coronavirus of copper, e.g., copper chelators, host cell copper transporter antagonists (for example, the CTR1 inhibitor, cimetidine, and steroids 4, 5 and 25 described in Kadioglu, O., et al., Molecular Docking Analysis of Steroid-based Copper Transporter 1 Inhibitors Anticancer Research 35: 6505-6508 (2015)), etc., that are useful in the invention are described herein, and include any therapeutically effective agent that sequesters copper, binds copper, chelates copper and/or otherwise deprives a copper-requiring coronavirus of copper, whether now known or later developed.

Preferred copper chelating agents are chelators of copper(I) and/or copper(II). Preferred copper(II) chelators are triethylenetetramine (trientine) and pharmaceutically acceptable salts thereof, including hydrochloride and succinate salts. Preferred triethylenetetramine salts are dihydrochloride and disuccinate salts. The disuccinate is salt is most preferred. The 350 mg, 400 mg, 500 mg, 600 mg and 700 mg fixed doses of triethylenetetramine disuccinate are most preferred as optimal doses and for daily dosing in amounts ranging, for example, from about 2400 mg to about 3000 mg.

Thus, in one aspect of the invention the copper depriving agent is triethylenetetramine or a pharmaceutically acceptable salt thereof, as noted, all of which can be used to deprive a coronavirus of copper. In another related aspect of the invention the copper depriving agent is cimetidine, a copper transport inhibitor that will reduce copper in viral host cells and inhibit coronavirus replication through copper deprivation.

In one embodiment, the agent effective to lower the copper values content in a subject or otherwise to deprive a coronavirus of copper comprises or consists essentially of or consists of an agent that may be selected from the group consisting of D-penicillamine; N-acetylpenicillamine; triethylenetetramine (also called TETA, TECZA, trien, triene and trientine), and pharmaceutically acceptable salts thereof; trithiomolybdate, tetrathiomolybdate, ammonium tetrathiomolybdate, choline tetrathiomolybdate; bis-choline tetrathiomolybdate (thiomolybdate USAN, trade name Decuprate), 2,2,2 tetramine tetrahydrochloride; 2,3,2 tetramine tetrahydrochloride; ethylenediaminetetraacetic acid salts (EDTA, a non-preferred non-specific metal binder, administered with care to avoid toxicity); diethylenetriaminetetraacetic acid (DPTA, a non-preferred non-specific metal binder, administered with care to avoid toxicity that is due to chelation of essential metals, such as Zn and Mn); 5,7,7′12,14,14′hexaxmethyl-1,4,8,11 tetraazacyclotretradecane; 1,4,8,11 tetraazacyclotretradecane, including cyclam S, cylams, and copper-chelating cyclam derivatives, e.g., Bn-cyclam-EtOH, oxo-cyclam-EtOH and oxo-Bn-cyclam-EtOH, (HOCH₂CH₂CH₂)₂(PhCH₂)₂Cyclam and (HOCH₂CH₂CH₂)₂(4-CF₃ PhCH₂)₂Cyclam; 1,4,8,11-tetraazacyclotetradecane-1,4,8,11-tetraacetic acid; 1,4,8,11-tetraazabicyclo[6.6.2]hexadecane; 4,11-bis(N,N-diethyl-amidomethyl)-1,4,8,11-tetraazabicyclo[6.6.2]hexadecane; 4,11-bis(amidoethyl)-1,4,8,11-tetraazabicyclo[6.6.2]hexadecane; melatonin; cyclic 3-hydroxymelatonin (30HM); N(1)-acetyl-N(2)-formyl-5-methoxykynuramine (AFMK); N(1)-acetyl-5-methoxykynuramine (AMK); N,N′-diethyldithiocarbamate; bathocuproinedisulfonic acid; bathocuprinedisulfonate; trimetazidine; triethylene tetramine tetrahydrochloride; 2,3,2-tetraamine; 1,10-orthophenanthroline; 3,4-dihydroxybenzoic acid; 2,2′-bicinchinonic acid; diamsar; 3,4′,5, trihydroxystilbene (resveratrol); mercaptodextran; disulfiram (Antabuse); sarcophagine; DiAmSar; diethylene triamine pentaacetic acid; and calcium trisodium diethylenetriaminepentaacetate; neocuproine; bathocuproine; and carnosine. Alternative names for trientine also include N,N′-Bis(2-aminoethyl)-1,2-ethanediamine; triethylenetetramine; 1,8-diamino-3,6-diazaoctane; 3,6-diazaoctane-1,8-diamine; and 1,4,7,10-tetraazadecane.

In another aspect of the invention the pharmaceutically acceptable salt is a polymorph of triethylenetetramine disuccinate that has a DSC extrapolated onset and peak melting temperatures of from between about 170° C. to about 190° C. In another aspect of the invention the pharmaceutically acceptable salt is a polymorph of triethylenetetramine disuccinate has a DSC extrapolated onset and peak melting temperature that are 180.05 and 179.91° C., respectively. In another aspect of the invention the pharmaceutically acceptable salt is a polymorph of triethylenetetramine disuccinate that has infrared peaks at wavenumbers at 3148, 1645, 1549, 1529, 1370, 1271, 1172, 1152, and 1033(±2 cm⁻¹). Triethylenetetramine disuccinate polymorphs are described in, for example, U.S. Pat. No. 8,067,641.

In another aspect of the invention the pharmaceutically acceptable salt is the Form I polymorph of triethylenetetramine dihydrochloride and is characterized by a DSC extrapolated onset and peak melting temperatures of between about 111° C. to 132° C. In another aspect of the invention the pharmaceutically acceptable salt is the Form I polymorph of triethylenetetramine dihydrochloride and is characterized by DSC extrapolated onset and peak melting temperatures that are 121.96 and 122.78° C., respectively. In another aspect of the invention the pharmaceutically acceptable salt is the Form I polymorph of triethylenetetramine dihydrochloride characterized by infrared peaks at wavenumbers 1043, 1116, 1300, 1328, 1557, 2833, 2895, 2902, and 3216(±2 cm⁻¹). See U.S. Pat. No. 8,067,641.

In another aspect of the invention the pharmaceutically acceptable salt is the Form II polymorph of triethylenetetramine dihydrochloride characterized by a DSC extrapolated onset and peak melting temperature of from between about 106° C. to about 126° C. In another aspect of the invention the pharmaceutically acceptable salt is the Form II polymorph of triethylenetetramine dihydrochloride characterized by a DSC extrapolated onset and peak melting temperatures that are 116.16 and 116.76° C., respectively. In another aspect of the invention the pharmaceutically acceptable salt is the Form II polymorph of triethylenetetramine dihydrochloride characterized by infrared peaks at wave numbers 1039, 1116, 1352, 1519, 2954, 2986, 3276, and 3298 (±2 cm⁻¹).

In another aspect of the invention the pharmaceutically acceptable salt is a polymorph of a triethylenetetramine disuccinate wherein the polymorph is a crystal having the structure defined by the co-ordinates of Table 3B found in U.S. Pat. No. 8,067,641. In another aspect of the invention the pharmaceutically acceptable salt is a polymorph of triethylenetetramine disuccinate wherein the polymorph is a crystal having the structure defined by the co-ordinates of Table 3C found in U.S. Pat. No. 8,067,641.

### Doses, Amounts and Concentrations

Copper-depriving compounds may be administered at dosages or a dosage to provide, if parenteral, at least about 120 mg/day in a human patient, and if oral, at least about 1200 mg/day in a human patient. Some oral doses of copper-depriving compounds may be administered at about 1200 to about 2400 mg/day. The total dosage may be given in single or divided dosage units (e.g., BID, TID, QID), and preferably maintain normal urine and/or plasma copper levels in a subject, or levels that do not fall below about 70% to 75% of normal. BID is presently preferred.

Other doses to treat human patients may range from about 10 mg to about 2000 mg/day of a virus copper-depriving compound. A typical dose may be about 100 mg to about 1500 mg/day of the compound. Other doses are from about 300 to about 2400 milligrams per day of the compound. Other doses include about 500 mg to about 1200 mg/day of the compound. Other doses are from about 600 to about 2400 milligrams per day of the compound. A dose may be administered once a day (QD), twice per day (BID), or more frequently, depending on the pharmacokinetic and pharmacodynamic properties, including absorption, distribution, metabolism, and excretion of the particular compound. In addition, toxicity factors may influence the dosage and administration regimen. When administered orally, the pill, capsule, or tablet may be ingested daily or less frequently for a specified period of time. The regimen may be repeated for a number of cycles of therapy.

In another aspect of the invention for treatment or prevention of coronavirus infections that require copper modulation an appropriate dosage level will generally be about 0.5 to about 50 mg or 100 mg per kg patient body weight per day which can be administered in single or multiple doses. Preferably, the dosage level will be about 1 to about 35 mg/kg per day; more preferably about 10 to about 35 mg/kg per day. A suitable dosage level may be about 0.5 to 25 mg/kg per day, about 1 to 10 mg/kg per day, or about 1 to 5 mg/kg per day. Within this range the dosage may be about 0.5 to about 1.0, 0.5 to 2.5 or 0.5 to 5 mg/kg per day. For oral administration, the compositions are preferably provided in the form of tablets containing about 100 to 1000 milligrams of the active ingredient, particularly about 100, 150, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, preferably once or twice per day.

Other exemplary doses include doses in the range of about 1 to 20 mg of active agent per kilogram of subject's body weight per day, preferably about 7 to about 18 mg/kg/day, or about 8 to 17 mg/kg/day, or about 10 to 15 mg/kg/day, up to about 35 mg/kg/day. The total dosage may be given in single or divided dosage units (e.g., preferably BID, but also TID or QID). The invention comprises administering the copper chelating agent to a mammal in an amount ranging from about 9 mg/kg to about 20 mg or 50 mg/kg per day. In another aspect of the invention the method comprises orally administering to a mammal a copper chelating agent in an amount ranging from about 1.2 to about 2.4 grams per day. Other doses and dose ranges are described below.

In one aspect of the invention the total daily dose administered ranges from 500 mg to 2500 mg of the succinic acid addition salt of triethylenetetramine.

In another aspect of the invention the composition comprises from 50 mg to 500 mg of the succinic acid addition salt of triethylenetetramine. In another aspect of the invention the composition comprises from 110 to 290 mg of the succinic acid addition salt of triethylenetetramine. In another aspect of the of the invention the composition comprises from 130 to 270 mg of the succinic acid addition salt of triethylenetetramine. In another aspect of the invention the composition comprises from 140 to 260 mg of the succinic acid addition salt of triethylenetetramine. In another aspect of the invention the composition comprises from 180 to 220 mg of the succinic acid addition salt of triethylenetetramine. In another aspect of the invention the composition comprises from 50 mg to 100 mg of the succinic acid addition salt of triethylenetetramine. In another aspect of the invention the composition comprises an amount of the succinic acid addition salt of triethylenetetramine selected from the group consisting of 50 mg, 110 mg, about 130 mg, 140 mg, 150 mg, 600 mg, 1200 mg, 2400 mg and 3000 mg. In another aspect of the invention the composition comprises an amount of the succinic acid addition salt of triethylenetetramine selected from the group consisting of 1.2 mg, 10 mg, 12 mg, 20 mg, 30 mg, and 40 mg.

We have discovered that triethylenetetramine disuccinate 1200 mg/day, given as 600 mg twice daily, would be expected to produce a significant cupruresis effect throughout the dosing interval with minimal side effects and negligible adverse effects on serum copper levels or other laboratory test parameters. We further discovered that fixed doses of triethylenetetramine disuccinate for optimal dosing and bioavailability are about 400 mg, about 500 mg, about 600 mg and about 700 mg of triethylenetetramine disuccinate. Exemplary effective amounts are described herein, and include doses in the range of from about 2400 mg per day to about 3000 mg per day given as multiple fixed doses of triethylenetetramine disuccinate comprising or consisting essentially of about 350 mg, 400 mg, about 500 mg, about 600 mg and/or about 700 mg.

In one aspect, fixed doses of triethylenetetramine disuccinate are about 400 mg, 500 mg, 600 mg and 700 mg. The fixed triethylenetetramine disuccinate doses may be used in methods of the invention to provide daily doses, including doses of from about 2400 mg to about 3000 mg. A fixed 350 mg dose of triethylenetetramine disuccinate is also provided. In another aspect of the invention, articles of manufacture comprising these fixed doses of triethylenetetramine disuccinate are provided. Capsules comprising these fixed doses of triethylenetetramine disuccinate are preferred.

### Manufacture

Copper-depriving agents, such as copper transporter antagonists and copper chelating agents, including for example triethylenetetramine dihydrochloride or triethylenetetramine disuccinate, suitable for use in the present invention can be purchased from commercial sources or can be prepared according to art known methods.

Two-component slow release preparations of a copper-depriving agent (e.g., a copper chelator agent) and an antiviral or anti-inflammatory agent in tablets or capsules are preferred, most preferably in capsules.

Copper chelator agents may be obtained from known manufacturing sources or synthesized using methods know in the art. Some copper chelators are manufactured using methods described in U.S. Pat. No. 9,556,123, which describes the synthesis of triethylenetetramines and useful intermediates in their production. U.S. Pat. No. 8,912,362 describes and claims isolated triethylenetetramine hydrochloride and dihydrochloride salts of varying purity, including 95% pure, 96% pure, 97% pure, 98% pure, 99% pure and 100% pure. It also claims isolated triethylenetetramine salts with a purity of greater than about 99% pure and less than 10 ppm of heavy metals.

U.S. Pat. No. 8,394,992 describes a useful process for preparing triethylenetetramine dihydrochloride, comprising: (a) reacting triethylenetetramine tetrahydrochloride with a base in a solvent to produce triethylenetetramine and chloride salt; (b) removing said chloride salt from solution (e.g., by precipitation or filtration); (c) reacting the triethylenetetramine with about 2 equivalents of concentrated hydrochloric acid to form triethylenetetramine dihydrochloride; and (d) adding an alcohol to the solution and precipitating triethylenetetramine dihydrochloride. Bases include sodium methoxide and sodium ethoxide. Solvents include ethanol, methanol and tert-butylmethylether. Alcohols include ethanol, methanol and isopropanol. Yields can be greater than 86% and up to 100%. The '992 patent also claims thermodynamic polymorphs of crystalline triethylenetetramine dihydrochloride.

U.S. Pat. No. 8,067,641 describes the preparation of polymorphs of triethylenetetramine disuccinate, including various Form I and Form II polymorphs, as well as pharmaceutical compositions with substantially pure polymorphs.

### Pharmaceutical Preparations

Also provided are pharmaceutical preparations. As used herein, pharmaceutical preparations mean compositions that include a copper-depriving agent (e.g., a copper chelator agent, for example, triethylenetetramine dihydrochloride or triethylenetetramine disuccinate), alone or together with an antiviral and/or an anti-inflammatory agent, present in a pharmaceutically acceptable vehicle. The term “pharmaceutically acceptable” has the meaning set forth above and includes those vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal.

The choice of excipient will be determined in part by the active ingredient, as well as by the particular method used to administer the composition. Accordingly, there is a wide variety of suitable formulations of the pharmaceutical composition of the present invention.

In one aspect, the present disclosure provides pharmaceutical preparation wherein the copper-depriving agent is a copper(II) chelator, e.g., triethylenetetramine dihydrochloride or triethylenetetramine disuccinate, alone or together with an antiviral and/or anti-inflammatory agent. The dosage form of the copper chelator agent in the methods of the present invention can be prepared by combining the copper chelator agent with one or more pharmaceutically acceptable diluents, carriers, adjuvants, and the like in a manner known to those skilled in the art of pharmaceutical formulation. The dosage form of the antiviral and/or anti-inflammatory agent employed in the methods of the present invention can be prepared by combining the antiviral and/or anti-inflammatory agent, with one or more pharmaceutically acceptable diluents, carriers, adjuvants, and the like in a manner known to those skilled in the art of pharmaceutical formulation. In some cases, and preferably, the dosage form of the copper-depriving agent and the dosage form of the antiviral and/or anti-inflammatory agent, are combined in a single composition, as noted.

Compositions may take the form of any standard known dosage form, including those mentioned above, and including tablets, pills, capsules, semisolids, powders, sustained release formulation, solutions, suspensions, elixirs, aerosols, liquids for injection, gels, creams, transdermal delivery devices (for example, a transdermal patch), inserts such as CNS inserts, or any other appropriate compositions. Persons of ordinary skill in the art to which the invention relates will appreciate the most appropriate dosage form having regard to the nature of the condition to be treated and the active agent to be used without any undue experimentation. Various doses and dose ranges are described herein. It should be appreciated that one or more of the copper-depriving agents and an antiviral and/or anti-inflammatory agent, for example, may be formulated into a single composition. In certain embodiments, preferred dosage forms include an injectable solution, a topical formulation, and an oral formulation.

In addition to standard diluents, carriers and/or excipients, a composition in accordance with the invention may be formulated with one or more additional constituents, or in such a manner, so as to enhance the activity or bioavailability of the copper-depriving agents (alone or together with an antiviral and/or anti-inflammatory agent), help protect the integrity or increase the half-life or shelf life thereof, enable slow release upon administration to a subject, or provide other desirable benefits, for example. For example, slow release vehicles include macromers, poly(ethylene glycol), hyaluronic acid, poly(vinylpyrrolidone), or a hydrogel. By way of further example, the compositions may also include preserving agents, solubilizing agents, stabilizing agents, wetting agents, emulsifying agents, sweetening agents, coloring agents, flavoring agents, coating agents, buffers and the like. Those of skill in the art to which the invention relates will readily identify further additives that may be desirable for a particular purpose.

Compounds of the invention may be administered by a sustained-release system. Suitable examples of sustained-release compositions include semi-permeable polymer matrices in the form of shaped articles, e.g., films, or microcapsules. Sustained-release matrices include polylactides (U.S. Pat. No. 3,773,919; EP 58,481), copolymers of L-glutamic acid and gamma-ethyl-L-glutamate, poly(2-hydroxyethyl methacrylate), ethylene vinyl acetate, or poly-D-(−)-3-hydroxybutyric acid (EP 133,988). Sustained-release compositions also include a liposomally entrapped compound. Liposomes containing copper chelating agents (alone or together with an antiviral and/or anti-inflammatory agent) may be prepared by known methods, including, for example, those described in: DE 3,218,121; EP 52,322; EP 36,676; EP 88,046; EP 143,949; EP 142,641; Japanese Pat. Appln. 83-118008; U.S. Pat. Nos. 4,485,045 and 4,544,545; and EP 102,324. Ordinarily, the liposomes are of the small (from or about 200 to 800 Angstroms) unilamellar type in which the lipid content is greater than about 30 mole percent cholesterol, the selected proportion being adjusted for the most efficacious therapy. Slow release delivery using PGLA nano- or microparticles, or in situ ion activated gelling systems may also be used, for example.

As noted, it is contemplated that a pharmaceutical composition in accordance with the invention may be formulated with additional active ingredients or agents which may be of therapeutic or other benefit to a subject in particular instances. Persons of ordinary skill in the art to which the invention relates will be able to identify suitable additional active ingredients having regard to the description of the invention herein and nature of the disorder to be treated.

Therapeutic formulations for use in the methods and preparation of the compositions of the present invention can be prepared by any methods well known in the art of pharmacy. See, for example, Gilman et al. (eds.) GOODMAN AND GILMAN'S: THE PHARMACOLOGICAL BASES OF THERAPEUTICS (8th ed.) Pergamon Press (1990); and Remington, THE SCIENCE OF PRACTICE AND PHARMACY, 20th Edition. (2001) Mack Publishing Co., Easton, Pa.; Avis et al. (eds.) (1993) PHARMACEUTICAL DOSAGE FORMS: PARENTERAL MEDICATIONS Dekker, N.Y.; Lieberman et al. (eds.) (1990) PHARMACEUTICAL DOSAGE FORMS: TABLETS Dekker, N.Y.; and Lieberman et al. (eds.) (1990) PHARMACEUTICAL DOSAGE FORMS: DISPERSE SYSTEMS Dekker, N.Y. Compositions may also be formulated in accordance with standard techniques as may be found in such standard references as Gennaro A R: Remington: The Science and Practice of Pharmacy, 20.sup.th ed., Lippincott, Williams & Wilkins, 2000, for example.

In some embodiments, nanoemulsion particles are used that have an average diameter of less than or equal to about 900 nm, less than or equal to about 800 nm, less than or equal to about 700 nm, less than or equal to about 600 nm, less than or equal to about 500 nm, less than or equal to about 400 nm, less than or equal to about 300 nm, less than or equal to about 200 nm, less than or equal to about 150 nm, less than or equal to about 100 nm, or less than or equal to about 50 nm. In some embodiments, nanoemulsion particles have an average diameter of about 400 nm.

Nanoemulsions have been used as topical antimicrobial formulations as well as vaccine adjuvants. Prior teachings related to nanoemulsions are described in, for example, U.S. Pat. Nos. 6,015,832; 6,506,803; 6,559,189; 6,635,676; and 7,314,624.

In some embodiments, the nanoemulsion further comprises at least one quaternary ammonium compound. In some embodiments, the nanoemulsion further comprises a surfactant. In some embodiments, the nanoemulsion further comprises a nonionic surfactant. In some embodiments, the nanoemulsion further comprises an organic solvent. In some embodiments, the nanoemulsion further comprises an antimicrobial. In some embodiments, the nanoemulsion further comprises an oil, which may be selected from the group consisting of soybean oil, mineral oil, avocado oil, squalene oil, olive oil, canola oil, corn oil, rapeseed oil, safflower oil, sunflower oil, fish oils, flavor oils, cinnamon bark, coconut oil, cottonseed oil, flaxseed oil, pine needle oil, silicon oil, essential oils, water insoluble vitamins, other plant oil, or a combination thereof. In some embodiments, the nonionic surfactant is: (a) a polysorbate, a poloxamer, or a combination thereof; and/or (b) selected from the group consisting of polysorbate 20, polysorbate 21, polysorbate 40, polysorbate 60, polysorbate 61, polysorbate 65, polysorbate 80, polysorbate 81, and polysorbate 85; and/or (c) selected from the group consisting of poloxamer 407, poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, Poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, poloxamer 105 Benzoate, and poloxamer 182 Dibenzoate; and/or (d) selected from the group consisting of an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis[imidazoyl carbonyl]), nonoxynol-9, Bis(polyethylene glycol bis[imidazoyl carbonyl]), Brij 35, Brij 56, Brij 72, Brij 76, Brij 92V, Brij 97, Brij 58P, Cremophor EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether. Igepal CA-630, Igepal CA-630, Methyl-6-O—(N-heptylcarbamoyl)-alpha-D-glucopyranoside, Nonaethylene glycol monododecyl ether, N—N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether. Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Span 20, Span 40, Span 60, Span 65, Span 80, Span 85, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tergitol, Tergitol, Type TMN-10, Tergitol, Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton X-114, Triton X-165, Triton X-305, Triton X-405, Triton X-45, Triton X-705-70, Tyloxapol, n-Undecyl beta-D-glucopyranoside, semi-synthetic derivatives thereof, and any combinations thereof; and/or (e) Generally Recognized as Safe (GRAS) by the US Food and Drug Administration.

In some embodiments, the quaternary ammonium compound is: (a) monographed by the US FDA as an antiseptic for topical use; (b) benzalkonium chloride (BZK); and/or (c) BZK present in a concentration of from about 0.05% to about 0.40%; and/or (d) BZK present in a concentration of from about 0.10% to about 0.20%; and/or (e) BZK present in a concentration of about 0.13%; and/or (f) cetylpyridimium chloride (CPC); and/or (g) CPC present in a concentration of from about 0.05% to about 0.40%; and/or (h) CPC present in a concentration of from about 0.15% to about 0.30%; and/or (i) CPC present in a concentration of about 0.20%; and/or (j) benzethonium chloride (BEC); and/or (k) BEC present in a concentration of from about 0.05% to about 1%; and/or (1) BEC present in a concentration of from about 0.10% to about 0.30%; and/or (m) BEC present in a concentration of about 0.20%; and/or (n) dioctadecyl dimethyl ammonium chloride (DODAC); and/or (o) DODAC present in a concentration of from about 0.05% to about 1%; and/or (p) DODAC present in a concentration of from about 0.10% to about 0.40%; and/or (q) DODAC present in a concentration of about 0.20%; and/or (r) octenidine dihydrochloride (OCT); and/or (s) OCT present in a concentration of from about 0.05% to about 1%; and/or (t) OCT present in a concentration of from about 0.10%, to about 0.400; and/or (u) OCT present in a concentration of about 0.20%.

In some embodiments, the composition used in the method further comprises a therapeutic agent, and optionally wherein the therapeutic agent is: (a) an antimicrobial agent; an antiviral agent; an antifungal agent; vitamin; homeopathic agent; anti-inflammatory agent; keratolytic agent; antipruritic agent; pain medicine; steroid; anti-acne drug; macromolecule; small, lipophilic, low-dose drug; naloxone; or an antigen; and/or (b) naloxone; and/or (c) is recognized as being suitable for transdermal, intranasal, mucosal, vaginal, or topical administration or application; and/or (d) has low oral bioavailability but is suitable for nasal administration when formulated into a nanoemulsion; and/or (e) is a lipophilic agent having poor water solubility; and/or (f) present within a nanoemulsion is formulated for intranasal administration, where the therapeutic agent when not present in a nanoemulsion is conventionally given via IV or IM due to the desire for fast onset of action or because of the difficulty in obtaining suitable bioavailability with other modes of administration; and/or (g) is a small, lipophilic, low-dose drug; and/or (h) is a macromolecule; and/or (i) selected from the group consisting of a penicillin, a cephalosporin, cycloserine, vancomycin, bacitracin, miconazole, ketoconazole, clotrimazole, polymyxin, colistimethate, nystatin, amphotericin B, chloramphenicol, a tetracycline, erythromycin, clindamycin, an aminoglycoside, a rifamycin, a quinolone, trimethoprim, a sulfonamide, zidovudine, ganciclovir, vidarabine, acyclovir, poly(hexamethylene biguanide), terbinafine, and a combination thereof; and/or (j) a homeopathic agent; and/or (k) a vitamin; and/or (l) an antigen; and/or (m) an anti-inflammatory agent; and/or (n) an anti-inflammatory agent which is a steroid or a non-steroidal anti-inflammatory drug; and/or (o) an anti-inflammatory agent which is a steroid which is selected from the group consisting of clobetasol, halobetasol, halcinonide, amcinonide, betamethasone, desoximetasone, diflucortolone, fluocinolone, fluocinonide, mometasone, clobetasone, desonide, hydrocortisone, prednicarbate, triamcinolone, and a pharmaceutically acceptable derivative thereof; and/or (p) an anti-inflammatory agent which is a non-steroidal anti-inflammatory drug selected from the group consisting of aceclofenac, aspirin, celecoxib, clonixin, dexibuprofen, dexketoprofen, diclofenac, diflunisal, droxicam, etodolac, etoricoxib, fenoprofen, flufenamic acid, flurbiprofen, ibuprofen, indomethacin, isoxicam, ketoprofen, ketorolac, licofelone, lornoxicam, loxoprofen, lumiracoxib, meclofenamic acid, mefenamic acid, meloxicam, nabumetone, naproxen, nimesulide, oxaprozin, parecoxib, phenylbutazone, piroxicam, rofecoxib, salsalate, sulindac, tenoxicam, tolfenamic acid, tolmetin, or valdecoxib.

In some embodiments, the composition has been: (a) autoclaved and composition retains its structural and/or chemical integrity following autoclaving; (b) formulated in nasal or inhalation dosage form; and/or (c) formulated into a dosage form selected from the group consisting of dry powder, nasal spray, aerosol, nasal swab; and/or (d) formulated liquid dosage form, solid dosage form, or semisolid dosage form; (e) formulated into a nasal or dermal swab impregnated or saturated with the copper-depriving agent.

Particular formulations of the invention are in a solid form, particularly tablets or capsules for oral administration.

Particular formulations of the invention are in a form for nasal administration, e.g., nanoemulsion. Other formulations of the invention are in the form of a transdermal patch.

### Articles of Manufacture/Kits

The invention also includes an article of manufacture, or “kit”, containing materials useful for treating the coronavirus diseases and infections described herein is provided. The kit comprises a container comprising a copper chelating agent(s), copper sequestering agent(s), copper depriving agent(s) and/or an anti-inflammatory agent (e.g., dexamethasone or another corticosteroid (prednisone, methylprednisolone)), preferably, carprofen or celecoxib, which inhibit a key enzyme in the replication and transcription of the virus responsible for COVID-19 (and/or another antiviral agent). The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, e.g., bottles, vials, syringes, blister pack, etc. The container may be formed from a variety of materials such as glass or plastic. The container may hold a copper chelating agent(s), copper sequestering agent(s), copper depriving agent(s) and/or an anti-inflammatory agent, e.g., carprofen, or a formulation thereof which is effective for treating the coronavirus condition and may have a sterile access port (e.g., the container may be an intravenous solution bag or a vial having a stopper pierceable by a hypodermic injection needle). The container may also be a package containing a composition in the form of a tablet or capsule, the latter being preferred, where the copper chelating agent(s), copper sequestering agent(s), copper depriving agent(s) and/or an anti-inflammatory agent are provided as separate compositions or together in combination in a single composition, e.g., in combined tablet or capsule, or nasal endotracheal, endosinusial, intrabronchial, intracavernous, intrasinal or transmucosal formulation. The label or package insert indicates that the composition(s) is/are used for treating a coronavirus condition or infection, such COVID-19 disease, or more of the other symptoms described herein.

### EXAMPLES

The inventions are related to and describe methods relating to discoveries surrounding copper and mechanisms leading to inhibition of coronavirus replication and transcription, e.g., COVID-19, by depriving a coronavirus of copper, in whole or in part. The beneficial effect of administration of copper-depriving compounds, e.g., chelating compounds, in the treatment of coronavirus infection is described.

The following examples are put forth so as to provide those of ordinary skill in the art with a complete disclosure and description of how to make and use the present invention, and are not intended to limit the scope of what the inventors regard as their invention, nor are they intended to represent that the experiments below are all or the only experiments.

Example 1 is an in vivo animal study on distribution of the copper-depriving compounds, in this case, the preferred copper chelator triethylenetetramine disuccinate. We have shown that, when administered to experimental animals, triethylenetetramine disuccinate enters organs including the upper respiratory tract, the lungs, and the heart. These are sites of coronavirus replication and are the same organs that are attacked by coronavirus infection in humans, including the coronaviruses leading to COVID-19 disease. Significant tissue penetration was found throughout 42 different body tissues, including the brain, heart, lung and liver, etc. in both species. In the male pigmented rat, maximum tissue concentrations of radioactivity were evenly distributed between the 1 h and 8 h time points. Highest levels of radioactivity were seen in the various tissues that included the lung at 1 hr post-dose, with penetration to the lung continuing for a full 8 hours. At 24 h post-dose elimination was on-going in the male pigmented rat with approximately half of the measured tissues having levels of radioactivity below the limit of quantification. At 72 h post-dose, elimination of radioactivity in the male pigmented rat was almost complete with approximately 65% of tissues below the limit of quantification.

Evaluation of the use of copper-depriving compounds in treating coronavirus infection, and the requirements for copper in coronavirus replication, is described in Examples 2-12.

Example 2-4 describe methods for preparing and isolating a coronavirus, e.g., the SARS-CoV-2 coronavirus that leads to COVID-19 disease.

Examples 5-9 describe the viral RNA quantification and minigenome assays and other methods that can be used to evaluate viral activity and the efficacy of copper-depriving compounds, which can then be passed to toxicology and eventually to a clinical trial for safety and tolerability in which a composition comprising a copper-depriving agent is studied in human volunteers.

Examples 10 and 11 describe microscopy methods for imaging.

Example 12 describes the use of copper-depriving compounds (and, optionally, other anti-viral agents) to attenuate coronaviruses, including SARS-CoV-2, in susceptible cells.

Novel dosing regimens for the copper-depriving compound triethylenetetramine disuccinate are described in Example 13 and 14, which describe human population pharmacokinetic and pharmacodynamic modeling of triethylenetetramine, its two major metabolites, and copper excretion after oral 2-way crossover administration of triethylenetetramine disuccinate and triethylenetetramine dihydrochloride to healthy adult volunteers in a human clinical study, and reveals the bioavailability of triethylenetetramine disuccinate.

The Example 15 study demonstrates that triethylenetetramine disuccinate will have good absorption in humans (estimated at approximately 70%).

The results demonstrate that the exposure to a copper-depriving agent, e.g., a copper chelator agent, may be done safely and can be expected lead to prevention and treatment of coronavirus disease, e.g., COVID-19 disease, by depriving a coronavirus and related copper-dependent respiratory viruses, e.g., the SARS-CoV-2 virus, of copper.

### Example 1

The Quantitative Tissue Distribution of Total Radioactivity in the Rat Following Single Oral Administration of [2-¹⁴C] PX811019

Study objectives: The objective of this in vivo study was to provide quantitative information on the tissue distribution of the copper-depriving compound triethylenetetramine disuccinate following oral administration to male albino and male pigmented rats. Whole body phosphor imaging (WBPI) was carried out on whole body sections taken from three albino male rats sacrificed at 1, 3, 8 and 24 h post-dose and from one pigmented rat at 1, 8, 24, 72, 168 and 336 h post-dose. Tissue radioactivity concentrations within individual sections were quantified using a phosphor imager system. Annotated images of the selected sections at each time point were produced using a supplementary software package designed for this purpose. Terminal blood samples were taken from all animals immediately prior to sacrifice and were analyzed for radioactivity. The study was conducted in compliance with Good Laboratory Practice (GLP).

Test substance: [2-¹⁴C] PX811019 (radiolabeled triethylenetetramine disuccinate), supplied by Selcia as a solid at a radiochemical purity of 99.6%. The authenticity and radiochemical purity were determined at Aptuit prior to use in this study, using high performance liquid chromatography (HPLC).

Analytical reagents: Liquid scintillant, Gold Star™, was obtained from Meridian (Epsom, Surrey, UK) and Ultima Gold™ and Permafluor®E+ were obtained from PerkinElmer LAS (UK) Ltd. The CO₂ absorbing solution Carbo Sorb®E was also obtained from PerkinElmer LAS (UK) Ltd. Unless otherwise stated, all other analytical reagents were of at least standard analytical laboratory reagent grade and were obtained principally from VWR International Ltd (Poole, Dorset, UK) and Sigma-Aldrich Company Ltd (Poole, Dorset, UK). De-ionised water was prepared in-house.

Animals: A sufficient number of animals
were obtained for use in the study:

| | | |
|---|---|---|
| Species: | Rat | Rat |
| Strain: | Sprague-Dawley | Lister Hooded |
| Sex: | Male | Male |

| | | | | |
|---|---|---|---|---|
| Age: | 6 | weeks | 6 | weeks |

| | | |
|---|---|---|
| Number of animals: | 12 | 6 |

| | | | | |
|---|---|---|---|---|
| Acclimatisation: | 7 | days | 7 | days |

| | | |
|---|---|---|
| Source: | Harlan UK Limited | Harlan UK Limited |

Animals were identified uniquely by tail marking with indelible ink and animal numbers were allocated arbitrarily. All studies are conducted in accordance with the Act Animals (Scientific Procedures) Act 1986, with UK Home Office Guidance on the implementation of the Act and with all applicable Codes of Practice for the care and housing of laboratory animals. The facility used was fully accredited by the Association for Assessment and Accreditation of Laboratory Animal Care (AAALAC). The health of the animals was assessed prior to the study, and all animals were healthy and deemed suitable for experimental use.

Study design: Each rat received a single oral administration of [2-¹⁴C] PX811019 at a target dose level of 10 mg/kg free-base. Quantitative whole-body phosphor imaging (QWBPI) was carried out on whole body sections taken from three albino male rats sacrificed at 1, 3, 8 and 24 h post-dose and from one pigmented rat at 1, 8, 24, 72, 168 and 336 h post-dose. Tissue radioactivity concentrations within individual sections were quantified and annotated, and representative images of the selected sections at each time point were produced. Terminal blood samples were taken from all animals immediately prior to sacrifice and analyzed for radioactivity.

Results

Radiochemical purity: Prior to use, the radiochemical purity of [2-¹⁴C] PX811019 was determined to be 97.7% with a single impurity of 0.9%. The mean radioactive concentration of the formulation was determined to be 22.2 μCi/g (0.82 MBq/g) at the time of dosing and the mean specific radioactivity of formulated [2-¹⁴C] PX811019 was determined to be 22.2 μCi/mg (0.82 MBq/mg).

Doses administered: The doses administered ranged between 9.96 and 10.2 mg/kg for PX811019. The radioactive dose ranged from 8.14 to 8.32 MBq/kg.

Animal observations and environmental control: No animal observations were made during the in-life phase that could be attributed to the administration of [2-¹⁴C] PX811019. During the in-life phase, the temperature and relative humidity in the room housing the animals ranged between 20° C. to 22° C. and 67% to 90%, respectively.

Tissue distribution of radioactivity following oral administration: Mean tissue concentrations of radioactivity in male albino rats following oral administration of [2-¹⁴C]PX811019 at a target dose level of 10 mg/kg free-base are presented in Table 1.

TABLE 1
Concentrations of radioactivity in organs and tissues at various
times following single oral administration of [2-¹⁴C]
PX811019 to male albino rats at a target dose level of 10 mg/kg
free-base (results expressed as ng equiv/g)

| Tissue/organ | 1 h | 3 h | 8 h | 24 h |
|---|---|---|---|---|
| LOQ (limit of | 126 | 110 | 118 | 126 |
| Adrenal cortex | 512 | 448 | 160 | blq |
| Adrenal medulla | 587 | 446 | 326 | 151 |
| Bone | 543 | 483 | 214 | blq |
| Bone marrow | 520 | 751 | 528 | 214 |
| Brain | blq | blq | blq | blq |
| Brown fat | 665 | 586 | 591 | blq |
| Caecum contents | 211281 | 437329 | 126104 | 3208 |
| Caecum wall | 22306 | 28979 | 10713 | 321 |
| Cardiac blood | 694 | 290 | 215 | blq |
| Cardiac muscle | 382 | 344 | 324 | blq |
| Epididymis | 388 | 253 | 218 | blq |
| Eye humour | blq | blq | blq | blq |
| Eye lens | blq | blq | blq | blq |
| Fur | 613 | 295 | 253 | blq |
| Harderian gland | 289 | 320 | 535 | 212 |
| Kidney cortex | 6323 | 6617 | 3783 | 1036 |
| Kidney medulla | 6760 | 4444 | 3733 | 1054 |
| Large intestine contents | blq | blq | 473267 | 9204 |
| Large intestine wall | 822 | 482 | 4964 | 605 |
| Liver | 3034 | 2231 | 1416 | 260 |
| Lung | 801 | 507 | 392 | blq |
| Nasal mucosa | blq | 332 | blq | blq |
| Pancreas | 563 | 795 | 643 | blq |
| Pineal body | 370 | 655 | 533 | 166 |
| Pituitary gland | 866 | 470 | 562 | 234 |
| Preputial gland | 411 | 519 | 380 | 201 |
| Prostate | 5361 | 6632 | 920 | blq |
| Seminal vesicles | 319 | 564 | 472 | 186 |
| Skeletal muscle | 224 | 234 | 190 | blq |
| Small intestine contents | 268536 | 96883 | 13207 | 685 |
| Small intestine wall | 14301 | 6739 | 4897 | 1144 |
| Spinal cord | blq | blq | blq | blq |
| Spleen | 520 | 573 | 547 | 231 |
| Stomach contents | 317629 | 188232 | 2601 | blq |
| Stomach wall | 2624 | 1353 | 841 | 215 |
| Submaxillary salivary | 652 | 1018 | 917 | blq |
| Testes | 196 | 159 | 184 | blq |
| Thymus | 453 | 796 | 845 | 438 |
| Thyroid gland | 433 | 671 | 683 | 287 |
| Urine | 29573 | 104341 | 15185 | 520 |
| White fat | 206 | blq | blq | blq |
| Whole blood # | 527 | 243 | 114 | 60.6 |

blq below limit of qualifications
#value obtained by sample combustion

Tissue concentrations of radioactivity in male pigmented rats following oral administration of [2-¹⁴C] PX811019 at a target dose level of 10 mg/kg free-base are presented in Table 2.

TABLE 2
Concentrations of radioactivity in organs and tissues at various times following
single oral administration of [2-¹⁴C] PX811019 to male pigmented rats
at a target dose level of 10 mg/kg free-base (results expressed as ng equiv/g)

| Tissue/organ | 1 h | 8 h (114M) | 24 h | 72 h | 168 h | 336 h (118M) |
|---|---|---|---|---|---|---|
| LOQ (limit of | 125 | 123 | 114 | 129 | 112 | 106 |
| Adrenal cortex | 238 | 342 | 222 | blq | blq | blq |
| Adrenal medulla | 292 | 440 | 178 | 147 | blq | blq |
| Bone | 152 | 253 | blq | blq | blq | blq |
| Bone marrow | 292 | 368 | 210 | blq | blq | blq |
| Brain | blq | blq | blq | blq | blq | blq |
| Brown fat | 285 | 255 | 136 | blq | blq | blq |
| Caecum contents | blq | 251695 | 5099 | 226 | blq | blq |
| Caecum wall | 452 | 11591 | 1428 | 381 | blq | blq |
| Cardiac blood | 410 | 151 | blq | blq | blq | blq |
| Cardiac muscle | 241 | 151 | blq | blq | blq | blq |
| Epididymis | 303 | 151 | blq | blq | blq | blq |
| Eye Choroid layer | 324 | 157 | 128 | 144 | blq | blq |
| Eye humour | blq | blq | blq | blq | blq | blq |
| Eye lens | blq | blq | blq | blq | blq | blq |
| Fur (non-pigmented) | 249 | 277 | blq | blq | blq | blq |
| Fur (pigmented) | 307 | 299 | blq | blq | blq | blq |
| Harderian gland | 143 | 590 | 163 | 130 | blq | blq |
| Kidney cortex | 4797 | 1293 | 565 | 277 | blq | blq |
| Kidney medulla | 2648 | 1236 | 727 | 233 | blq | blq |
| Large intestine contents | ns | 424462 | 9630 | 381 | blq | blq |
| Large intestine wall | 227 | 3563 | 1369 | 192 | blq | blq |
| Liver | 1299 | 615 | 242 | blq | blq | blq |
| Lung | 480 | 194 | blq | blq | blq | blq |
| Nasal mucosa | blq | 134 | blq | blq | blq | blq |
| Pancreas | 347 | 334 | 130 | 200 | blq | blq |
| Pineal body | 255 | blq | blq | 133 | blq | blq |
| Pituitary gland | 291 | 298 | 205 | 145 | blq | blq |
| Preputial gland | 185 | 419 | 136 | blq | blq | blq |
| Prostate | ns | 277 | blq | blq | blq | blq |
| Seminal vesicles | 295 | 193 | 127 | 162 | blq | blq |
| Skeletal muscle | blq | 173 | blq | blq | blq | blq |
| Small intestine contents | 448683 | 22920 | 1771 | 167 | blq | blq |
| Small intestine wall | 4639 | 2488 | 748 | 169 | blq | blq |
| Spinal cord | blq | blq | blq | blq | blq | blq |
| Spleen | 184 | 905 | 192 | 143 | blq | blq |
| Stomach contents | 268321 | 30411 | blq | blq | blq | blq |
| Stomach wall | 1292 | 2985 | 240 | 169 | blq | blq |
| Submaxillary salivary | 448 | 651 | 142 | blq | blq | blq |
| Testes | blq | blq | blq | blq | blq | blq |
| Thymus | 268 | 572 | 204 | 155 | blq | blq |
| Thyroid gland | 375 | 472 | 161 | 134 | blq | blq |
| Urine | 6174 | 790 | 654 | 527 | blq | blq |
| White fat | 136 | blq | blq | blq | blq | blq |
| Whole blood # | 332 | 79.9 | 61.0 | 52.0 | 31.7 | 25.0 |

blq below limit of quantification
# value obtained by sample combustion

Tissue:blood ratios in male albino and pigmented rats are presented in Table 3 and Table 4, respectively.

TABLE 3
Tissue: blood ratios at various times following single
oral administration of [2-¹⁴C] PX811019 to male
albino rats at a target dose level of 10 mg/kg free-base

| Tissue/organ | 1 h | 3 h | 8 h | 24 h * |
|---|---|---|---|---|
| Adrenal cortex | 0.74 | 1.54 | 0.74 | nc |
| Adrenal medulla | 0.85 | 1.54 | 1.52 | 2.49 |
| Bone | 0.78 | 1.67 | 1.00 | nc |
| Bone marrow | 0.75 | 2.59 | 2.46 | 3.53 |
| Brain | nc | nc | nc | nc |
| Brown fat | 0.96 | 2.02 | 2.75 | nc |
| Cardiac blood | 1.00 | 1.00 | 1.00 | nc |
| Cardiac muscle | 0.55 | 1.19 | 1.51 | nc |
| Epididymis | 0.56 | 0.87 | 1.01 | nc |
| Eye humour | nc | nc | nc | nc |
| Eye lens | nc | nc | nc | nc |
| Fur | 0.88 | 1.02 | 1.18 | nc |
| Harderian gland | 0.42 | 1.10 | 2.49 | 3.50 |
| Kidney cortex | 9.11 | 22.8 | 17.6 | 17.1 |
| Kidney medulla | 9.74 | 15.3 | 17.4 | 17.4 |
| Liver | 4.37 | 7.69 | 6.59 | 4.29 |
| Lung | 1.15 | 1.75 | 1.82 | nc |
| Nasal mucosa | nc | 1.14 | nc | nc |
| Pancreas | 0.81 | 2.74 | 2.99 | nc |
| Pineal body | 0.53 | 2.26 | 2.48 | 2.74 |
| Pituitary gland | 1.25 | 1.62 | 2.61 | 3.86 |
| Preputial gland | 0.59 | 1.79 | 1.77 | 3.32 |
| Prostate | 7.72 | 22.9 | 4.28 | nc |
| Seminal vesicles | 0.46 | 1.94 | 2.20 | 3.07 |
| Skeletal muscle | 0.32 | 0.81 | 0.88 | nc |
| Spinal cord | nc | nc | nc | nc |
| Spleen | 0.75 | 1.98 | 2.54 | 3.81 |
| Submaxillary salivary gland | 0.94 | 3.51 | 4.27 | nc |
| Testes | 0.28 | 0.55 | 0.86 | nc |
| Thymus | 0.65 | 2.74 | 3.93 | 7.23 |
| Thyroid gland | 0.62 | 2.31 | 3.18 | 4.74 |
| White fat | 0.30 | nc | nc | nc |

Tissue: blood ratios calculated using cardiac blood values
nc not calculable
* calculated using whole blood value

TABLE 4
Tissue: blood ratios at various times following single
oral administration of [2-¹⁴C] PX811019 to male
pigmented rats at a target dose level of 10 mg/kg free-base

| Tissue/organ | 1 h | 8 h | 24 h * | 72 h * | 168 h | 336 h |
|---|---|---|---|---|---|---|
| Adrenal cortex | 0.58 | 2.26 | 3.64 | nc | nc | nc |
| Adrenal medulla | 0.71 | 2.91 | 2.92 | 2.83 | nc | nc |
| Bone | 0.37 | 1.68 | nc | nc | nc | nc |
| Bone marrow | 0.71 | 2.44 | 3.44 | nc | nc | nc |
| Brain | nc | nc | nc | nc | nc | nc |
| Brown fat | 0.70 | 1.69 | 2.23 | nc | nc | nc |
| Cardiac blood | 1.00 | 1.00 | nc | nc | nc | nc |
| Cardiac muscle | 0.59 | 1.00 | nc | nc | nc | nc |
| Epididymis | 0.74 | 1.00 | nc | nc | nc | nc |
| Eye Choroid layer | 0.79 | 1.04 | 2.10 | 2.77 | nc | nc |
| Eye humour | nc | nc | nc | nc | nc | nc |
| Eye lens | nc | nc | nc | nc | nc | nc |
| Fur (non- | 0.61 | 1.83 | nc | nc | nc | nc |
| Fur (pigmented) | 0.75 | 1.98 | nc | nc | nc | nc |
| Harderian gland | 0.35 | 3.91 | 2.67 | 2.50 | nc | nc |
| Kidney cortex | 11.7 | 8.56 | 9.26 | 5.33 | nc | nc |
| Kidney medulla | 6.46 | 8.19 | 11.9 | 4.48 | nc | nc |
| Liver | 3.17 | 4.07 | 3.97 | nc | nc | nc |
| Lung | 1.17 | 1.28 | nc | nc | nc | nc |
| Nasal mucosa | nc | 0.89 | nc | nc | nc | nc |
| Pancreas | 0.85 | 2.21 | 2.13 | 3.85 | nc | nc |
| Pineal body | 0.62 | nc | nc | 2.56 | nc | nc |
| Pituitary gland | 0.71 | 1.97 | 3.36 | 2.79 | nc | nc |
| Preputial gland | 0.45 | 2.77 | 2.23 | nc | nc | nc |
| Prostate | nc | 1.83 | nc | nc | nc | nc |
| Seminal vesicles | 0.72 | 1.28 | 2.08 | 3.12 | nc | nc |
| Skeletal muscle | nc | 1.15 | nc | nc | nc | nc |
| Spinal cord | nc | nc | nc | nc | nc | nc |
| Spleen | 0.45 | 5.99 | 3.15 | 2.75 | nc | nc |
| Submaxillary | 1.09 | 4.31 | 2.33 | nc | nc | nc |
| Testes | nc | nc | nc | nc | nc | nc |
| Thymus | 0.65 | 3.79 | 3.34 | 2.98 | nc | nc |
| Thyroid gland | 0.91 | 3.13 | 2.64 | 2.58 | nc | nc |
| White fat | 0.33 | nc | nc | nc | nc | nc |

Tissue: blood ratios calculated using cardiac blood values
nc not calculable
* calculated using whole blood value

As likely following oral administration, high concentrations of radioactivity were found in the gastrointestinal tract (large intestine contents 473267 ng equiv/g at 8 h post-dose, caecum contents 437329 ng equiv/g at 3 h post-dose, stomach contents 317629 ng equiv/g at 1 h post-dose and small intestine contents 268536 ng equiv/g at 1 h post-dose). Urinary concentrations were also high with the highest level being 104341 ng equiv/g observed at 3 h post-dose. All values given above are for the male albino rats and are also generally representative of the pigmented animals.

In the male albino rat, maximum tissue concentrations of radioactivity were achieved, in which cellular uptake was measured included approximately 44% of the measured tissues, at 1 h post-dose with a further 30% at 3 h post-dose. At 1 h post-dose, it appeared that absorption was on-going in approximately half of the tissues. Highest levels of radioactivity were seen in the kidney medulla, prostate, liver, pituitary gland and lung (6760, 5361, 3034, 866 and 801 ng equiv/g, respectively), compared to a cardiac blood concentration of 694 ng equiv/g. The liver and kidney medulla had their maximum concentration at this time point. At 3 h post-dose, highest levels of radioactivity were associated with the prostate, kidney cortex, kidney medulla, liver and the submaxillary salivary gland (6632, 6617, 4444, 2231 and 1018 ng equiv/g, respectively), compared to a cardiac blood concentration of 290 ng equiv/g. Although levels of radioactivity in the prostate appeared to be high at this time point it was considered that this was a result of urinary contamination. At 8 h post-dose, with the exception of the Harderian gland (535 ng equiv/g), thymus (845 ng equiv/g) and thyroid gland (683 ng equiv/g), which had their maximum tissue concentration at this time, radioactivity concentrations in tissues were lower than their maximum values. Highest levels of radioactivity were associated with the kidney cortex, kidney medulla, liver, prostate, thymus and thyroid gland (3783, 3733, 1416, 920, 845 and 683 ng equiv/g, respectively), compared to a cardiac blood concentration of 215 ng equiv/g. At 24 h post-dose, elimination of radioactivity was on-going with approximately 80% of tissues at or below the limit of quantification. Highest levels of radioactivity were observed in the kidney medulla, kidney cortex and thymus (1054, 1036 and 438 ng equiv/g, respectively).

Tissue:blood ratios in the male albino rat, where calculable, ranged between 0.28 (testes) and 22.9 (prostate) at 1 and 3 h post-dose, respectively. The majority of tissues had a tissue:blood ratio of greater than 1 with highest ratios calculated in the prostate (22.9), kidney cortex (22.8), kidney medulla (17.4) and liver (7.69) at 3, 3, 8 and 3 h post-dose, respectively. As detailed above, it was deemed that prostate levels appeared high in relation to a urinary contamination. However, the majority of tissue:blood ratios at 1 h post-dose were less than 1, with ratios tending to increase with time. This may indicate a slower uptake and release by the tissues compared with blood.

Distribution of radioactivity in the male pigmented rat was similar to that observed in the male albino rats. In the male pigmented rat, maximum tissue concentrations of radioactivity were evenly distributed between the 1 h and 8 h time points. Maximum levels were achieved in approximately 40% of the measured tissues at 1 h post-dose, with a further 45% at 8 h post-dose. At 1 h post-dose, absorption was considered to be on-going in approximately half of the tissues. Highest levels of radioactivity were seen in the kidney cortex, kidney medulla and liver (4797, 2648 and 1299 ng equiv/g, respectively), compared to a cardiac blood concentration of 410 ng equiv/g. The liver and kidney had their maximum concentration at this time point. At 8 h post-dose, highest levels of radioactivity were associated with the kidney cortex, kidney medulla, spleen, submaxillary salivary gland, liver, Harderian gland and thymus (1293, 1236, 905, 651, 615, 590 and 572 ng equiv/g, respectively), compared to a cardiac blood concentration of 151 ng equiv/g. At 24 h post-dose elimination was on-going with approximately half of the measured tissues having levels of radioactivity below the limit of quantification. Highest levels were associated with the kidney medulla and kidney cortex (727 and 565 ng equiv/g, respectively). At 72 h post-dose, elimination of radioactivity was almost complete with approximately 65% of tissues below the limit of quantification. Highest levels of radioactivity were observed in the kidney cortex, kidney medulla and pancreas (277, 233 and 200 ng equiv/g, respectively).

At 168 h post-dose, elimination appeared to be complete with all levels of radioactivity in tissues below the limit of quantification.

Tissue:blood ratios in the male pigmented rat, where calculable, ranged between 0.33 (white fat) and 11.9 (kidney medulla) at 1 and 24 h post-dose, respectively. The majority of tissues had a tissue:blood ratio of greater than 1 with highest ratios calculated in the kidney medulla (11.9), kidney cortex (11.7), spleen (5.99) and liver (4.07) at 24, 1, 8 and 8 h post-dose, respectively. However, the majority of tissue:blood ratios at 1 h post-dose were less than 1, with ratios tending to increase with time. This may indicate a slower uptake and release by the tissues compared with blood.

Radioactivity levels in blood measured by QWBPI were compared to values obtained by sample combustion of blood samples taken immediately prior to sacrifice. Similar trends and order of magnitude were evident between values obtained by QWBPI measurement and values obtained by sample combustion. Blood levels at 1 h post-dose were 694 and 527 ng equiv/g by QWBPI quantification and by sample combustion, respectively, for the male albino rats and 410 and 332 ng equiv/g by QWBPI quantification and by sample combustion, respectively, for the male pigmented animals

In summarizing certain aspects of this study, it is noted that, following oral administration, high concentrations of radioactivity were observed in the contents of the gastrointestinal tract. In the male albino rat, absorption of radioactivity was rapid with measurable levels of radioactivity present in the majority of tissues at 1 h post-dose. Maximal levels of radioactivity were reached in approximately 44% of tissues at 1 h post-dose and a further 30% of tissues reached maximum levels at 3 h post-dose. The highest tissue concentrations of radioactivity were attained in the kidney medulla, prostate, kidney cortex and liver (6760, 6632, 6617 and 3034 ng equiv/g, respectively), at 1, 3, 3 and 1 h post-dose, respectively. Although levels of radioactivity in the prostate appeared to be high it was considered that this was a result of urinary contamination.

Concentrations and distribution of radioactivity in the male pigmented rat were similar to those seen in the male albino rat. Maximum levels of radioactivity were reached in approximately 40% of tissues at 1 h post-dose and a further 45% of tissues reached maximum levels at 8 h post-dose. Highest levels were associated with the kidney cortex, kidney medulla and liver (4797, 2648 and 1299 ng equiv/g, respectively), all at 1 h post-dose.

Conclusions: The distribution and concentration of total radioactivity in the male albino rats and the male pigmented rats were similar. Binding to the melanin of pigmented tissues was not evident.

PX811019 is rapidly absorbed and distributed with nearly half of tissues having maximum concentrations of radioactivity at 1 h post-dose, but with absorption on-going in approximately half of the tissues. Tissue:blood ratios in the majority of tissues reached values greater than 1 after the 1 h time point, which may indicate a slow uptake and release by the tissues compared with blood.

Tissues associated with biotransformation and elimination (e.g., liver and kidney) and secretory glands (e.g., pancreas, submaxillary salivary gland, thymus and thyroid gland) tended to have higher concentrations of radioactivity.

Elimination of radioactivity from tissues was generally rapid, with decreased tissue levels observed at 24 h post-dose and appeared to be complete in the majority of tissues by 72 h.

### Example 2—Virus, Culture Cells, Limiting Dilution, and Virus Isolation

Coronaviruses, for example, the COVID-19 virus (SARS-CoV-2) may be obtained from a trusted source, e.g., the CDC or NIH. Basic research resources including the distribution of viral isolates and reagents are available through the National Institute of Allergy and Infectious Diseases (NIAID)-funded BEI Resources Repository.

Alternatively, the virus may be isolated from clinical specimens, and Vero E6 or Vero CCL-81 cells used for isolation and initial passage. Vero E6 cells are the best choice for amplification and quantification, but both Vero cell types support amplification and replication of coronaviruses, including SARS-CoV-2. Nasopharyngeal (NP) and oropharyngeal (OP) swab specimens are used to obtain clinical specimens for virus isolation.

For isolation, limiting dilution, and passage 1 of the virus, 50 μL of serum-free DMEM (i.e., Dulbecco's Modified Eagle Medium without fetal bovine serum) is pipetted into columns 2-12 of a 96-well tissue culture plate, and 100 μL of clinical specimens are then pipetted into column 1 and serially diluted 2-fold across the plate. Vero cells in DMEM are then trypsinized and resuspended containing 10% fetal bovine serum, 2× penicillin/streptomycin, 2× antibiotics/antimycotics, and 2× amphotericin B at a concentration of 2.5×10⁵ cells/mL. 100 μL of cell suspension is added directly to the clinical specimen dilutions and mixed gently by pipetting. Inoculated cultures are grown in a humidified 37° C. incubator in an atmosphere of 5% CO₂ and observed for cytopathic effects (CPEs) daily. Standard plaque assays for SARS-CoV-2 are used, which are based on SARS-CoV and Middle East respiratory syndrome coronavirus (MERS-CoV) protocols (see Sims A C, et al. Release of severe acute respiratory syndrome coronavirus nuclear import block enhances host transcription in human lung cells. J Virol. 2013; 87:3885-902; Josset L, et al. Cell host response to infection with novel human coronavirus EMC predicts potential antivirals and important differences with SARS coronavirus. MBio. 2013; 4:e00165-13). When CPEs are observed, cell monolayers are scraped with the back of a pipette tip. 50 μL of viral lysate is used for total nucleic acid extraction for confirmatory testing and sequencing. 50 μL of virus lysate is also used to inoculate a well of a 90% confluent 24-well plate. The sequenced genome of stock COVID-19 virus is compared to those recorded in government SARS-CoV-2 sequence resources database, e.g., at the NIH.

### Example 3—Sars-Cov Production and Infection

Vero E6 cells (American Type Culture Collection, Manassas, Va.) are propagated in 75 cm² cell culture flasks in growth medium consisting of medium 199 (Sigma, St Louis, Mo.) supplemented with 10% fetal calf serum (FCS; Biological Industries, Kibbutz Beit Haemek, Israel). SARS-CoV 2003VA2774 (an isolate from a SARS patient in Singapore), which has been previously sequenced, may be used for propagation in Vero E6 cells. Briefly, 2 mL of stock virus is added to a confluent monolayer of Vero E6 cells and incubated at 37° C. in 5% CO₂ for 1 h; 13 mL of medium 199 supplemented with 5% FCS is then added. The cultures are incubated at 37° C. in 5% CO₂, and the supernatant is harvested after 48 h; in >75% of cultures, inhibition of CPE (3+) in each well is observed with an inverted microscope. The supernatant is clarified at 2,500 rpm and then divided into aliquots, placed in cryovials, and stored at −80° C. until use.

### Example 4—Virus Handling and Titration

All virus culture and assays are carried out in a biosafety level-3 laboratory, according to applicable safety conditions set out. Virus titer in the frozen culture supernatant is determined by using a plaque assay. Briefly, 100 μL of virus in 10-fold serial dilution is added, in duplicates, to a monolayer of Vero E6 cells in a 24-well plate. After 1 h of incubation at 37° C. in 5% CO₂, the viral inoculum is aspirated, and 1 mL of carboxymethylcellulose overlay with medium 199, supplemented with 5% FCS, is added to each well. After 4 days of incubation, the cells are fixed with 10% formalin and stained with 2% crystal violet. The plaques are counted visually, and the virus titer in plaque-forming units per mL (PFU/mL) is calculated.

### Example 5—Cytopathic Endpoint Assay

The protocol used is adapted from Al-Jabri A A, Wigg M D and Oxford J S Initial in vitro screening of drug candidates for their potential antiviral activities. In: Mahy, B W J, Kangro H O, editors. Virology methods manual. London: Academic Press Ltd; 1996. p. 293-356, and all drugs are tested in quadruplicate. Briefly, 100 μL of serial 10-fold dilutions of the drugs are incubated with 100 μL of Vero E6 cells, giving a final cell count of 20,000 cells per well in a 96-well plate. The incubation period is 1 h at 37° C. in 5% CO₂, except for the interferons, which are incubated overnight with the cells. Ten μL of virus at a concentration of 10,000 PFU/well are then added to each of the test wells. The plates are incubated at 37° C. in 5% CO₂ for 3 days and observed daily for CPE. The end point is the drug dilution that inhibited 100% of the CPE (CIA₁₀₀) in quadruplicate wells. To determine cytotoxicity, 100 μL of serial 10-fold dilutions of the drugs are incubated with 100 μL of Vero E6 cells, giving a final cell count of 20,000 cells per well in a 96-well plate, without viral challenge. The plates are then incubated at 37° C. in 5% CO₂ for 3 days and examined for toxicity effects by using an inverted microscope.

### Example 6—Plaque Reduction Assay

Trypsinized Vero E6 cells are resuspended in growth medium and preincubated with interferons (serial fivefold dilution) in quadruplicate wells in 24-well plates. The next day, the medium are aspirated, and 100 μL of virus are added to each well at a titer of 100 PFU/well. After incubation for 1 h, the virus inoculum are aspirated, and a carboxymethylcellulose overlay containing maintenance medium and the appropriate interferon concentration are added. After 4 days' incubation, the plates are fixed and stained as described previously. The number of plaques are then counted visually, and the concentration of drug that inhibits 50% of plaques in each well (IC₅₀) are determined. Results are plotted in Microsoft Excel, and a polynomial of order of three are used to approximate the data and extrapolate IC₅₀ and IC₉₅ values.

### Example 7—Viral RNA Quantification

Control Vero E6 cells and Vero E6 cells treated with either Cu (copper), T (triethylenetetramine) or TTM (ammonium tetrathiomolybdate) are infected at multiplicity of infection (MOI)=1 or higher, and at predetermined times are washed with phosphate buffered saline (PBS). Lysates are harvested in buffer RLT and RNAs extracted by RNeasy kit (Qiagen, Valencia, Calif.). Viral RNA is quantified by qRT-PCR, using SYBR green based detection. Reverse-transcription and PCR reactions are performed in one tube with the iTaq kit (BioRad, Hercules, Calif.), in a BioRad CFX96 thermocycler. Primers for the viral RNA are available to researchers on the CDC website and elsewhere. Statistical significance is assessed by paired two-tailed t-test, p<0.05.

### Example 8—Viral Minigenome Assay

The minigenome (MG) system, also known as minireplicon or MG technology, is considered as a complementary and powerful tool for exploring the life cycle of a virus during infection. In evaluating coronoavirus activity, viral polymerase activity may also be assessed using an experimentally optimized minigenome assay with viral polymerase expression vectors, a vRNA firefly luciferase reporter construct (minigenome), and Renilla luciferase expression plasmid as an internal transfection control. Cells are then transfected with VPOL, minigenome, and Renilla plasmids, using the FuGENE HD transfection reagent (Promega), following the manufacturer's recommendations. 24 hours after the second transfection, cells are harvested and assayed using the Dual Luciferase Reporter Assay (Promega) on a BioTek Synergy HT reader.

### Example 9—Viral Protein Quantification

In another embodiment, an assay is developed to build a targeted peptide quantification assay for the detection of two of the viral proteins from SARS-CoV-2. Briefly, proteins are extracted from the same samples harvested for viral RNA quantification, above. Extractions from buffer RLT are performed using the iced acetone method described by the manufacturer (Qiagen). Proteins are separated by denaturing SDS polyacrylamide gel electrophoresis, and transferred to PVDF (Pall Corp., Pensacola, Fla.). Immunoblotting is performed with a monoclonal antibody to the nucleocapsid of the COVID-19 virus (ViroStat, Westbrook, Me.), and peroxidase conjugated secondary antiserum. Blots are imaged with Supersignal substrate (ThermoFisher Scientific, Carlsbad, Calif.), on a Cell Biosciences FluorChem HD2. Consistent loading is monitored by Coomassie Brilliant Blue R-250 (Amresco, Solon, Ohio) staining of the post-transfer gel.

### Example 10—Immunofluorescence Microscopy

CuCl₂ concentrations are 10 μM for this experiment. Treated A549 cells are infected at MOI=1. At 12 hours post infection (h.p.i.), cells are washed with PBS, fixed in 4% paraformaldehyde, and permeabilized with 0.1% saponin. Samples are probed with primary antisera using sheep anti-TGN46 (Serotec), rabbit anti-ATP7A, or anti-NP monoclonal AA5H, in PBS with 0.05% Tween 20 and 3% bovine serum albumin. Secondary antisera conjugated to Alexa Fluor 488, 532, or 647 are used for visualization, and mounted in VectaShield with DAPI (Vector Laboratories, Burlingame, Calif.). Images are captured at room temperature with a Leica DM6000 B microscope with a 63× oil immersion objective, numerical aperture=1.4, and a Photometrics (Tucson, Ariz.) CoolSNAP MYO camera. Software for capture and deconvolution is Leica Application Suite X (LAS X) and image placement Adobe Illustrator.

### Example 11—Transmission Electron Microscopy

Treated A549 cells are infected at MOI=5. At 16 h.p.i., cells are washed with PBS and fixed in 2.5% glutaraldehyde (Electron Microscopy Sciences, Fort Washington, Pa.) and 0.1 M cacodylate, pH 7.4. Cells are then embedded in Eponate 12 resin, cut into 80-nm sections, and stained with 5% uranyl acetate and 2% lead citrate at the Emory Robert P. Apkarian Integrated Electron Microscopy Core. After sample preparation, grids are imaged at 75 kV using a Hitachi H-7500 transmission electron microscope.

### Example 12—Copper and Chelator Treatments

The effects of copper-depriving agents on coronaviruses are evaluated as described herein and optionally with the use of procedures set out in Examples 5 (Cytopathic Endpoint Assay), Example 6 (Plaque Reduction Assay), Example 7 (Viral RNA Quantification), Example 8 (Viral Minigenome Assay) and Example 9 (Viral Protein Quantification). Briefly, SARS-CoV-2 virus-infected Vero E6 cells are treated with (1) 50 μM CuCl₂ (Acros Organics, Morris Plains, N.J.), and with copper-depriving agents (2) 10 μM ammonium tetrathiomolybdate (TTM; Sigma-Aldrich, St. Louis, Mo.), or (3) 10 μM triethylenetetramine (T, ≥97.0%; Sigma-Aldrich, St. Louis, Mo.), preferably triethylenetetramine disuccinate, by supplementing normal growth medium and inoculums, beginning at 24 hours prior to subsequent treatments, i.e. infection. T is a copper²⁺-selective chelator. TTM is an efficient chelator of bioavailable copper. Ammonium tetrathiomolybdate acts to interfere with intestinal uptake of copper when administered with meals and binds plasma copper when taken between meals. It also removes copper from metallothioneins and can form insoluble copper complexes that are deposited in liver.

Intracellular copper concentrations in complete lysates of untreated (control) and (1) 50 μM CuCl₂, (2) 10 μM TTM and (3) 10 μM T, treatment of infected cells is assessed by inductively coupled plasma mass spectrometry (ICP-MS) elemental analysis. Cytotoxicity of CuCl₂, T, and TTM on cell viability is assayed by chemiluminescent ATP quantitation (CellTiter-Glo; Promega, Madison, Wis.). No decrease in luminescence is expected to be observed below concentrations of CuCl₂, T or TTM at least 5-fold higher than used for the study. Additionally, the effect of these treatments on virion viability is assayed. Samples may also be evaluated using immunofluorescence microscopy and/or transmission electron microscopy, as described in Examples 10 and 11.

Samples are quantitated for infectious activity, viral replication, viral RNA or protein, and/or transcription using copper-depriving compounds (and/or anti-viral agents, set forth below) and the coronavirus methods described above.

### Copper-Depriving Agent

### 1. D-penicillamine

### 2. N-acetylpenicillamine

### 3. Triethylenetetramine Dihydrochloride

4. Triethylenetetramine disuccinate

### Copper-Depriving Agent+Anti-Viral Agent

### 5. Triethylenetetramine Dihydrochloride+Interferon β-1b

6. Triethylenetetramine Dihydrochloride+Interferon α-n1 and/or α-n3

### 7. Triethylenetetramine Dihydrochloride+Human Leukocyte Interferon α

### 8. Triethylenetetramine Disuccinate+Interferon β-1b

9. Triethylenetetramine Disuccinate+Interferon α-n1 and/or α-n3

### 10. Triethylenetetramine Disuccinate+Human Leukocyte Interferon α

### Example 13

### A Single Center, Randomized, Double-Blind, Single-Dose, 2-Way Crossover, Dose Escalation Study of the Pharmacokinetics and Pharmacodynamics of Triethylenetetramine Disuccinate (PX811019) Compared with Triethylenetetramine Dihydrochloride in Normal Healthy Volunteers

This human clinical study provides population pharmacokinetic and pharmacodynamic modeling of triethylenetetramine, its two major metabolites, and copper excretion after oral 2-way crossover administration of triethylenetetramine disuccinate and triethylenetetramine dihydrochloride to healthy adult volunteers.

The population PK analysis encompasses samples from a study (TETA doses 166, 499, 832 mg of free base in each of three cohorts) where each subject received triethylenetetramine disuccinate (PX811019) and triethylenetetramine dihydrochloride (Syprine®) in a 2-way crossover design. triethylenetetramine dihydrochloride (Syprine®) is a potent copper chelator, which was approved by the FDA in 1985 for the second line treatment of Wilson's Disease. Triethylenetetramine disuccinate (PX811019) is an alternative, superior salt form of triethylenetetramine, but its target dosing is unknown, and unknowable from the prior art.

A population pharmacokinetic/pharmacodynamic (PK/PD) model was used to describe the concentrations of triethylenetetramine (TETA), its two major metabolites (monoacetylated (MAT) and diacetylated (DAT) forms), and copper excretion in urine. The model with first-order absorption and two-compartment kinetics for TETA, catenary formations of MAT and DAT, and copper excretion directly controlled by TETA in plasma was further used to identify differences between studied TETA formulations by estimating absorption-related parameters separately for PX811019 and Syprine®. The influences of subject-specific covariates and dose on PK/PD parameters were examined based on standard chi square statistics. Population PK/PD modeling was performed using the NONMEM software.

Objectives: The objectives of this study were to compare the pharmacokinetic (PK) profiles and to determine the dosing relationship of triethylenetetramine (TETA) disuccinate (PX811019) relative to TETA dihydrochloride (Syprine®) and to characterize the pharmacodynamic (PD) profile of urinary copper excretion in response to the study drug.

Abridged PK/PD, Half-Life, Absorption Kinetics and Bioavailability Summary: In carrying out this study, it was discovered that the relative bioavailability of PX811019 compared to Syprine® equaled 74.5%. Differences between Syprine and PX811019 in lag-times (0.083 and 0.239 h) and absorption rate constants (1.74 and 1.19 h⁻¹) were observed. A covariance analysis did not identify major PK/PD differences related to dose, sex, weight, or renal function. The compound exhibited highly stable and consistent PK/PD profiles. Some dose-dependence of TETA distribution volume was found producing a somewhat longer half-life at the higher doses. Differences in absorption kinetics between forms were modest with lesser bioavailability (74.5%) of PX811019 compared to Syprine® evident. It was discovered that administration of about 134% of the dose of PX811019 would produce essentially identical plasma concentrations of TETA, MAT, and DAT and copper excretion rates as Syprine®. See also, Example 3.

Methodology: This study was a Phase 1, prospective, randomized, double-blind, dose escalation, 2-way crossover design. It was planned that up to four cohorts, with six subjects per cohort, were to be enrolled. PX811019 or Syprine® doses were to be administered to subjects within each cohort at approximately molar equivalent doses of TETA free base (approximately 166 to 167 mg free base per capsule). Cohort doing is shown in Table 5:

TABLE 5
Study Dose of TETA Dihydrochloride and TETA Disuccinate

| | TETA Dihydro- | TETA Di- | | |
|---|---|---|---|---|
| | chloride | succinate | Number | Approximate |
| | (Syprine ®) | (PX811019) | of | TETA |
| Cohort | Dose (mg) | Dose (mg) | Capsules | mg free base |
| 1 | 250 | 435 | 1 | 166 |
| 2 | 750 | 1305 | 3 | 499 |
| 3 | 1250 | 2175 | 5 | 832 |

Following completion of each cohort, the Sponsor and the Investigator reviewed the plasma concentration-time profiles of TETA and its acetyl metabolites [monoacetyl TETA (MAT) and diacetyl TETA (DAT)] as well as safety and tolerability data prior to approving escalation to the next cohort. Based upon analysis of interim PK data, the decision was made to stop enrollment after completion of three cohorts.

There were three visits to the clinic: a Screening Visit that occurred within 28 days prior to the first dose, and two Treatment Visits. Subjects were screened and enrolled based on medical history, clinical laboratory results, physical examination findings, vital signs assessments and resting 12-lead ECG evaluations. Eligible subjects were admitted to the research facility by 2000 h on the evening prior to dosing for each Treatment Visit. Following an overnight fast, subjects were randomized to receive a single oral dose of PX811019 or a single oral dose of Syprine® on Day 1 and the alternate treatment on Day 8. Subjects remained confined to the research facility for 48 hours following each dose (until the morning of Day 3 or Day 10).

Safety evaluations included adverse event (AE) assessments, physical examinations, clinical laboratory tests and vital sign (blood pressure and pulse rate) assessments. Blood samples for determination of plasma TETA, MAT and DAT levels were collected on Day 1 and Day 8 at Time 0 (within 30 min prior to dosing), 5, 15, 30, 60, 90, 120 min and thereafter at 3, 4, 5, 6, 8, 10 and 12 h post-dose, and then at 16, 20, 24, 30, 36, 42 and 48 h post-dose on Days 2-3 and Days 9-10. Urinary copper excretion was measured in urine collected on Day 1 and Day 8 at the following intervals: from −2-0 h pre-dose and from 0-2, 2-4, 4-6, 6-8, 8-10, 10-12 and 12-16 h post-dose and then at 16-20, 20-24, 24-30, 30-36, 36-42 and 42-48 h post-dose on Days 2-3 and Days 9-10.

Protocol Amendments: There was one protocol amendment during the study period. Protocol Amendment 1 extended the screening window from 14 to 28 days from the Screening Visit to dosing on Day 1 in order to allow sufficient time for review of safety and PK data prior to escalation to the next cohort.

Number of Subjects: A total of 18 subjects (9 male, 9 female) were enrolled and randomized to receive single oral doses of PX811019 and Syprine®.

Criteria for Inclusion: To be eligible, subjects had to complete an appropriately administered IRB-approved informed consent prior to performance of any study-related procedures, and be healthy adult males or females between the ages 18 and 60 years, inclusive, with a body mass index (BMI) between 18 and 30 kg/m², inclusive, and have normal renal function as calculated by a creatinine clearance >90 mL/min. Females of child-bearing potential had to have a negative pregnancy test at the Screening Visit and upon each admission to the research facility, be willing to use an effective means of birth control for four weeks prior to study medication administration, and be non-lactating. Males must have been willing to use effective barrier contraception for four weeks after study medication administration. Subjects were excluded if they were smokers, had a history of drug or alcohol abuse, had participated in a clinical research study within 30 days prior to the first dose of study medication or had donated 1 pint or more of blood within 56 days, or plasma within 14 days, prior to the first dose of study medication; if they used iron, copper or other dietary supplements within two weeks prior to the first dose of study medication or during the study; required prescription or over-the-counter medication or herbal or nutritional supplements within one week prior to first dose of study medication or during the study; had a history of systemic lupus erythematosus, sideroblastic anemia, dystonia, muscular spasms or myasthenia gravis, or a history of therapeutic anti-coagulation; had a known allergy to TETA or formulation excipients; had pulmonary abnormalities evident from clinical examination; or clinical laboratory results at the Screening Visit that indicated any of the following: a clinical diagnosis of iron deficiency based on levels of plasma iron, iron-binding capacity and ferritin, copper deficiency based on low levels of plasma copper or ceruloplasmin, abnormal liver function test results or a platelet count <100×10⁶/L.

Test Product, Dose, and Mode of Administration: TETA disuccinate (PX811019) was supplied as 435 mg capsules and TETA dihydrochloride (Syprine® @) was supplied as 250 mg capsules, with each capsule representing approximately equimolar doses of TETA free base. Capsules for the two formulations were similar in size and shape but were not identical in appearance. In order to preserve the integrity of the blind, subjects were administered study medication while blindfolded by a designated pharmacist or sub-investigator not otherwise involved in the conduct of the study and subjects were not allowed to directly handle the capsules. Capsules were administered at approximately 0800 h on Day 1 and Day 8, following an overnight fast, with 240 mL water.

Duration of Treatment: This study included a Screening Visit within 28 days prior to the first dose of study medication administration, and two Treatment Visits separated by 7 days, each of which required 3 consecutive overnight stays.

Criteria for Evaluation: The PK profiles of PX811019 and Syprine® were evaluated by analysis of plasma concentrations of TETA and its metabolites, MAT and DAT, following single oral doses of both formulations. Pharmacodynamic parameters were evaluated by determination of urine copper excretion following single oral doses of both formulations. Safety was evaluated by assessing the frequency of treatment-emergent adverse events (AEs), discontinuations due to AEs, physical examination findings, changes in vital signs and clinical laboratory test results.

Statistical Methods: PK parameters for plasma TETA, MAT and DAT concentration data (including Cₘₐₓ, Tₘₐₓ, AUC₀₋₂₄, AUC₀₋ₜ, AUC_{0-inf}, elimination phase t½ and effective t½) were analyzed by noncompartmental methods. The dosing relationships of the PX811019 and Syprine® were evaluated by examination of the plasma concentration time curves and Cₘₐₓ for TETA, MAT and DAT for both formulations and by calculating the ratio of the AUC₀₋ₜ values for plasma TETA based on equivalent molar doses of TETA free base. Summary statistics for pharmacokinetic parameters and average urinary Cu excretion were computed for each formulation. Geometric means were also computed for AUC₀₋₂₄, AUC₀₋ₜ, AUC_{0-inf}, and Cₘₐₓ. Summary statistics (mean, median, standard error, minimum and maximum) for plasma concentrations and urinary Cu excretion were computed for each formulation at appropriate sampling times.

Safety data, including adverse events, vital signs assessments, clinical laboratory evaluations and physical examinations are summarized by formulation and dose cohort. Adverse events were coded using the MedDRA dictionary. A by-subject adverse event data listing, including verbatim term, preferred term and system organ classification, as well as severity, relationship to treatment and action taken, is provided. Concomitant medications are listed by subject and coded using the WHO drug dictionary. Descriptive statistics (arithmetic mean, standard error, median, minimum and maximum) were calculated using SAS.

Results: A total of 18 eligible subjects (9 males and 9 females) between 20 and 48 years of age, were enrolled and randomized to receive study medication. Seventeen (94.4%) subjects completed the study and one (5.6%) subject in Cohort 3 discontinued due to an adverse event following administration of PX811019 during the first Treatment Visit.

Demographics: Enrolled subjects were representative of a healthy adult population, ranging from 20 to 48 years of age. The overall mean (SD) age of enrolled subjects was 34.3 (8.14) years and the race distribution was 4 (22.2%) White, 6 (33.3%) Black, 7 (38.9%) Latino/Hispanic and 1 (5.6%) American Indian/Alaskan Native. Mean (SD) height and weight were 169.5 (8.46) cm and 73.9 (11.41) kg, respectively, and mean (SD) BMI was 25.7 (3.27) kg/m.

### Safety Results:

Treatment-Emergent AEs: Five subjects reported treatment-emergent adverse events; 3 of 17 (17.6%) subjects reported an AE after receiving Syprine® and 2 of 18 (11.1%) subjects reported an AE after receiving PX811019. AEs reported following administration of Syprine® included headache, diarrhea and nausea. AEs reported following administration of PX811019 included headache, diarrhea and elevated liver enzymes. All AEs were mild or moderate in intensity, and resolved prior to discharge from the study, and no serious AEs were reported. One subject in Cohort 3 discontinued the study due to mild, reversible elevated liver enzymes following administration of PX811019 (2175 mg) during the first Treatment Visit.

Other Safety Assessments: No clinically significant hemodynamic effects attributable to study medication were observed based on sitting blood pressure and pulse rate.

No clinically significant changes in laboratory test parameters were observed, except for one subject receiving PX811019 (2175 mg) who was reported to have a mild, reversible elevation in liver enzymes, considered possibly related to study medication.

### Pharmacokinetic Results:

The mean pharmacokinetic parameters for TETA, MAT and DAT following single equimolar oral doses in Cohorts 1, 2 and 3 are listed below in Table 6, and are further described in Example 3:

TABLE 6
Summary of PK Parameters for TETA, MAT and DAT as obtained
from non-compartmental analysis. The half-lives derived from
the final PK/PD model estimates are shown for comparison.
Summary of PK Parameters for TETA, MAT and DAT in PK Population

| | Cohort 1 (N−=6) | | Cohort 2 (N−=6) | | Cohort 3 (N−=6) | |
|---|---|---|---|---|---|---|
| | PX811014 | Syprine ® | PX811019 | Syprine ® | PX811019 | Syprine ® |
| Parameter (unit | 435 mg | 250 mg | 1305 mg | 750 mg | 2175 mg | 1250 mg |

TETA

| | | | | | | |
|---|---|---|---|---|---|---|
| Cₘₐₓ (mg/L) | 292.3412 | 496.6128 | 1121.0355 | 1873.0383 | 1814.5398 | 3430.2492 |
| AUC₀₋₂₄ (mg/L · h) | 1090.0472 | 1515.2096 | 4173.2842 | 6376.9208 | 7037.9122 | 13275.2109 |
| AUC₀₋ₜ (mg/L · h) | 1089.6795 | 1541.5092 | 4391.2987 | 6644.2427 | 7398.6177 | 13740.1848 |
| AUC_{0-inf} (mg/L · h) | 1157.8173 | 1691.0937 | 4738.4486 | 6905.3857 | 7728.7437 | 14131.1832 |
| t½ (h) | 8.3944 | 18.7837 | 26.8652 | 22.9857 | 21.7903 | 23.9489 |
| t½ (alpha) | 1.16 | 1.16 | 1.14 | 1.14 | 1.44 | 1.44 |
| Predicted, h | | | | | | |
| t½ (beta) Predicted, | 18.2 | 18.2 | 28.0 | 28.1 | 36.6 | 36.7 |
| h | | | | | | |

MAT

| | | | | | | |
|---|---|---|---|---|---|---|
| Cₘₐₓ (mg/L) | 460.0520 | 507.4967 | 1042.0688 | 1370.9102 | 1373.4790 | 1555.2304 |
| AUC₀₋₂₄ (mg/L · h) | 3606.0350 | 4548.8755 | 7771.6814 | 10240.2013 | 11764.3074 | 14279.3749 |
| AUC₀₋ₜ (mg/L · h) | 3933.3491 | 4981.8145 | 8644.9491 | 11301.4154 | 13371.4157 | 16339.2152 |
| AUC_{0-inf} (mg/L · h) | 4134.0485 | 5337.3440 | 9239.8333 | 11981.3464 | 14373.6664 | 17493.6323 |
| t½ (h) | 15.8239 | 22.3079 | 17.8289 | 18.3246 | 17.0265 | 17.7415 |
| t½ Predicted, h | 3.52 | 3.52 | 3.82 | 3.82 | 4.60 | 4.60 |

DAT

| | | | | | | |
|---|---|---|---|---|---|---|
| Cₘₐₓ (mg/L) | 95.5918 | 126.8557 | 267.0867 | 406.6258 | 397.1342 | 475.1264 |
| AUC₀₋₂₄ (mg/L · h) | 874.1022 | 1141.6082 | 2234.9524 | 3352.6220 | 3250.9811 | 4413.2536 |
| AUC₀₋ₜ (mg/L · h) | 918.6399 | 1216.1421 | 2666.4082 | 3787.3474 | 4113.1406 | 5142.3914 |
| AUC_{0-inf} (mg/L · h) | 986.8079 | 1292.5907 | 2778.4138 | 3940.5216 | 4354.1000 | 5423.7201 |
| t½ (h) | 7.1751 | 8.3329 | 10.5466 | 11.6251 | 11.9974 | 12.3562 |
| t½ Predicted, h | 0.687 | 0.687 | 0.390 | 0.390 | 0.426 | 0.426 |

### PK of TETA:

The Cₘₐₓ ratios of TETA after administration of PX811019 versus Syprine® to subjects in Cohorts 1, 2 and 3 were 0.58, 0.59 and 0.55, respectively, and the AUC₀₋ₜ, and AUC_{0-inf} ratios after administration of PX811019 versus Syprine® were 0.66-0.68, 0.64-0.65, and 0.55 for subjects in Cohorts 1, 2 and 3, respectively. The AUC₀₋₂₄ ratios of TETA were also lower after PX811019 versus Syprine® for subjects in all three dose cohorts.

The mean elimination t½ of TETA after administration of PX811019 and Syprine® to subjects in Cohort 1 was 8.4 and 18.8 h, respectively, and ranged from 21.8 to 26.9 h following administration of PX811019 and Syprine® to subjects in Cohort 2 and 3. The effective t½ values were approximately one-third to one-fourth the elimination t½ values in the three dose cohorts and were not dependent on the formulation, with the exception of PX811019 in Cohort 1, which was approximately half as much (4.5 h versus 8.4 h). The median Tₘₐₓ values ranged between 1.25 h and 2.0 h for all three dose cohorts.

### PK of MAT:

The Cₘₐₓ ratios of MAT after administration of PX811019 versus Syprine® to subjects in Cohorts 1, 2 and 3 were 0.87, 0.75 and 0.91, respectively. The AUC₀₋ₜ, and AUC_{0-inf} ratios after administration of PX811019 versus Syprine® were 0.74-0.76, 0.74-0.75 and 0.84, respectively. The mean t½ values for MAT were 16 and 22 h following administration of PX811019 and Syprine®, respectively, to subjects in Cohort 1, and were 17-18 h following administration of PX811019 and Syprine® to subjects in Cohorts 2 and 3. Exposure to MAT, as measured by AUC, was approximately 2-3 times higher compared to TETA at all three dose levels. Cₘₐₓ of MAT was higher than Cₘₐₓ of TETA following administration of PX811019 and Syprine® to subjects in Cohort 1, but lower for subjects Cohort 3 for both formulations. The median Tₘₐₓ for MAT was 5.0-5.5 h for both formulations for subjects in all three dose cohorts, occurring later than the Tₘₐₓ for the parent compound.

### PK of DAT:

Cₘₐₓ of DAT was generally 2- to 3-fold lower than for TETA and 3- to 4-fold lower compared to MAT. The AUCs of DAT were also lower than for both the parent drug and MAT for both formulations. The Cₘₐₓ ratio of DAT for the PX811019 formulation versus Syprine® was between 0.71 (Cohort 1) and 0.88 (Cohort 3) while the AUC ratios ranged between 0.72 (Cohort 1) and 0.84 (Cohort 3). The median Tₘₐₓ value for DAT was similar to the Tₘₐₓ for MAT (5.0 to 6.0 h).

### Pharmacodynamic Results

The majority of cupriuresis occurred during the first 6 hours following dosing for all dose groups. The level of cupriuresis increased as the dose of Syprine® increased from 250 to 1250 mg. It was approximately the same at 435 and 1305 mg PX811019 and increased at the highest dose of 2175 mg.

### CONCLUSIONS

Single oral doses of Syprine® (250, 750, 1250 mg) and PX811019 (435, 1305, 2175 mg) were both safe and well-tolerated by these healthy adult male and female subjects.

Adverse events were reported in 3 (17.6%) subjects following administration of Syprine® and in 2 (11.1%) subjects following administration of PX811019, and included headache, nausea, diarrhea and elevated liver enzymes. Adverse events were either mild or moderate in intensity, and no serious adverse events were reported.

One subject discontinued study participation due to a mild, reversible increase in liver enzymes following treatment with 2175 mg PX811019.

No clinically significant hemodynamic effects attributable to study medication were observed based on sitting blood pressure and pulse rate.

No clinically significant changes in laboratory test parameters were observed, except for one subject receiving 2175 mg PX811019 who was reported to have a mild, reversible elevation in liver enzymes, considered possibly related to study medication.

The majority of cupriuresis occurred during the first 6 h following dosing for all dose groups and cupriuresis increased as dose levels increased. No clear difference in urinary excretion of copper due to formulation was apparent.

Cₘₐₓ of TETA was 41-45% lower following a single oral dose of the PX811019 formulation at all three dose levels tested compared to administration of equimolar doses of Syprine®.

AUC₀₋ₜ and AUC_{0-inf} were 34-45% lower following a single oral dose of the PX811019 formulation at all three dose levels compared to administration of equimolar doses of Syprine®.

Values of Cₘₐₓ and AUC for the metabolites MAT and DAT were lower following a single oral dose of the PX811019 formulation at all three dose levels compared to administration of equimolar doses of Syprine®.

### Overall Conclusions

Single oral doses of PX811019 (435, 1305, 2175 mg) and Syprine® (250, 750, 1250 mg) were safe and well-tolerated by these healthy adult male and female subjects. Adverse events were either mild or moderate in intensity and no serious adverse events were reported. One subject discontinued study participation due to a mild, reversible increase in liver enzymes following treatment with 2175 mg PX811019. Cₘₐₓ of TETA was 41-45% lower and AUC₀₋ₜ and AUC_{0-inf} of TETA were 34-45% lower following a single oral dose of the PX811019 formulation at the three dose levels tested compared to administration of equimolar doses of Syprine®. There was no clear difference in the urinary excretion of copper due to the formulation.

Triethylenetetramine disuccinate 1200 mg/day, given as 600 mg twice daily, would be expected to produce a significant cupruresis effect throughout the dosing interval with minimal side effects and negligible adverse effects on serum copper levels or other laboratory test parameters.

### Example 14

### Population Pharmacokinetic and Pharmacodynamic Modeling of Triethylenetetramine

The data analyzed in this report were obtained in the Example 13 study, a double-blind, dose escalation, 2-way crossover design study comparing TETA disuccinate (PX811019) and TETA dihydrochloride (Syprine®). The Example 13 study demonstrated that administration of TETA as the disuccinate salt results in lower exposure indices (Cₘₐₓ and AUC) of TETA and its metabolites. Population-based modeling is used here to compare the absorption kinetics and provide a more global assessment of relative bioavailability of the two salt forms of TETA in the context of the enacted Example 13 study design.

The Example 14 analysis applies a model-based population analysis to the data in order to obtain an integrated assessment of the pharmacokinetics of TETA, MAT, and DAT, to further assess the pharmacodynamics of urinary excretion of copper, to consider potential covariates with the PK/PD parameters such as sex, age and dose, and in comparing the PK/PD of Syprine® and PX811019 from Example 13, particularly in regard to bioavailability.

Study Analysis: A population PK/PD model for TETA and its metabolites was developed based on data obtained from the Example 13 Study (A Single Center, Randomized, Double-Blind, Single-Dose, 2-Way Crossover, Dose Escalation Study of the Pharmacokinetics and Pharmacodynamics of Triethylenetetramine Disuccinate (PX811019) Compared with Triethylenetetramine Dihydrochloride in Normal Healthy Volunteers) in which subjects were randomized to receive either a single oral dose of PX811019 or a single oral dose of Syprine® on Day 1 and the alternate treatment on Day 8. The data from three cohorts, with six subjects per cohort, were available. For one subject in cohort 3 only Day 1 PD data were available. The PX811019 or Syprine doses were administered to subjects within each cohort at approximately molar equivalent doses of TETA free base (approximately 166 to 167 mg free base per capsule) at the doses set forth in Table 5 in Example 13.

Blood samples for determination of plasma TETA, MAT and DAT concentrations were collected on Days 1 and 8 at Time 0 (within 30 min prior to dosing), 5, 15, 30, 60, 90, 120 min and thereafter at 3, 4, 5, 6, 8, 10, 12, 16 and 20 h post-dose, and then at 24, 30, 36, 42 and 48 h post-dose on Days 2-3 and Days 9-10. Urinary copper excretion was measured via urine collections on Days 1 and 8 at the following intervals: from −2-0 h (pre-dose) and from 0-2, 2-4, 4-6, 6-8, 8-10, 10-12, 12-16, 16-20 and 20-24 h post-dose and then at 24-30, 30-36, 36-42 and 42-48 h post-dose on Days 2-3 and Days 9-10.

### Population Pharmacokinetic/Pharmacodynamic Analysis

Data Handling for PK/PD Analysis: The PK samples were analyzed using a validated bioanalytical LC/MS/MS method for the simultaneous determination of triethylenetetramine and its two main metabolites in human serum. Triethylenetetramine (TETA) and two major TETA-derived metabolites were measured: N1-acetyltriethylenetetramine (MAT) and N1,N10-diacetyltriethylenetetramine (DAT). The assay LLOQ was 0.005 mg/L for TETA, MAT and DAT. The urine samples were collected for copper analysis, which served as the pharmacodynamic endpoint. The concentrations falling below the limit of quantification (BLQ) were handled using the Beal M3 method with the F-FLAG option. Beal, SL, Ways to fit a PK model with some data below the quantification limit. J Pharmacokinet Pharmacodyn. 2001, 28:481-504.

Population PK/PD Methods: Population nonlinear mixed-effect modeling was done using NONMEM (Version 7.3.0, Icon Development Solutions, Ellicott City, Md., USA) and the gfortran compiler 9.0. NONMEM runs were executed using Wings for NONMEM (WFN730, http://wfn.sourceforge.net). The Laplacian estimation method was used. The differential equations for the model were solved using ADVAN6 PREDPP subroutines. The NONMEM data processing and plots were performed in Matlab® Software version 7.0 (The MathWorks, Inc., Natick, Mass., USA).

The minimum value of the NONMEM objective function, typical goodness-of-fit diagnostic plots, and the evaluation of the precision of pharmacokinetic parameter and variability estimates were used to discriminate between various models during the model-building process.

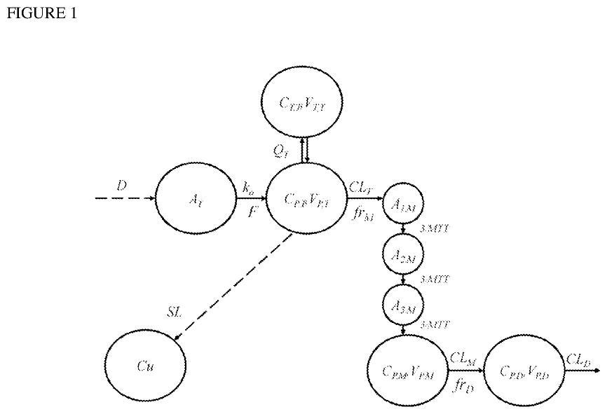

Population PK/PD Model: The PK/PD model (FIG. 1) used to describe TETA, MAT, and DAT concentrations and copper amounts excreted in urine is based in part on our findings in another human study (Study No. GC007-11: An open-label study to evaluate an effect of acetylation phenotype on triethylenetetramine dihydrochloride (GC811007) metabolism in healthy adult volunteers). It is a first-order absorption, two-compartment disposition model for TETA and catenary one-compartment disposition models for MAT and DAT. A series of transit compartments was used to describe the delay between the TETA and MAT concentrations. The following equations were used for the PK:

$$\begin{matrix}\frac{{dA}_{T}}{dt} = - {k}_{a} \cdot {A}_{T} & ( Equation 1 ) \\ {V}_{P , T} \cdot \frac{{dC}_{P , T}}{dt} = CONV \cdot F \cdot {F}_{P / S} \cdot {k}_{a} \cdot {A}_{T} - {Q}_{T} \cdot {C}_{P , T} + {Q}_{T} \cdot {C}_{T , T} - {CL}_{T} \cdot {C}_{P , T} & ( Equation 2 ) \\ {V}_{T , T} \cdot \frac{{dC}_{T , T}}{dt} = {Q}_{T} \cdot {C}_{P , T} - {Q}_{T} \cdot {C}_{T , T} & ( Equation 3 ) \\ \frac{{dA}_{1 , M}}{dt} = {fr}_{M} \cdot {CL}_{T} \cdot {C}_{P , T} - 3 / MTT \cdot {A}_{1 , M} & ( Equation 4 ) \\ \frac{{dA}_{2 , M}}{dt} = 3 / MTT \cdot ( {A}_{1 , M} - {A}_{2 , M} ) & ( Equation 5 ) \\ \frac{{dA}_{3 , M}}{dt} = 3 / MTT \cdot ( {A}_{2 , M} - {A}_{3 , M} ) & ( Equation 6 ) \\ {V}_{P , M} \cdot \frac{{dC}_{P , M}}{dt} = 3 / MTT \cdot {A}_{3 , M} \cdot \frac{188}{146} - {CL}_{M} \cdot {C}_{P , M} & ( Equation 7 ) \\ {V}_{P , D} \cdot \frac{{dC}_{P , D}}{dt} = {fr}_{D} \cdot {CL}_{M} \cdot {C}_{P , M} \frac{233}{188} - {CL}_{D} \cdot {C}_{P , D} & ( Equation 8 )\end{matrix}$$

The initial conditions of Equations 1-8 are: A_{T}(0)=D; C_{P,T}(0)=0; C_{T,T}(0)=0; C_{P,M}(O)=0; A_{1,M}(0)=0; A_{2,M}(0)=0; A_{3,M}(0)=0; and C_{P,D}(0)=0. The F denotes the presumed bioavailability of TETA (Syprine); F_{P/S} denotes relative bioavailability of PX811019 vs. Syprine; C_{P,T}, C_{P,M}, C_{P,D} are the concentrations of TETA, MAT and DAT in plasma; C_{T,T} is the concentration of TETA in the peripheral compartment; CL_{T}, CL_{M}, CL_{D} are the systemic clearances of TETA, MAT and DAT; Q_{T} is the distribution clearance of TETA; V_{P,T}, V_{P,M}, V_{P,D} are the volumes of distribution for TETA, MAT and DAT; V_{T,T} is the peripheral volume of distribution for TETA; MTT is the mean transit time accounting for the delay between TETA and MAT concentrations. The model was tested with 0 to 3 transit steps. The fr_{M}, and fr_{T} are fractions of TETA metabolized to MAT and MAT metabolized to DAT. The molecular masses for TETA, MAT and DAT (146, 188 and 233 g/mol) were used to convert mass changes between parent compound and metabolites (viz. TETA equivalents). CONV equaled 0.5721 (250/435) for PX811019 and 1 for Syprine and was used to convert mass of PX811019 to Syprine equivalents.

The model actual parameters generated were: CL_{T}/F, Q_{T}/F, V_{P,T}/F, V_{T,T}/F for TETA; CL_{M}/F/f_{rM} and V_{P,M}/F/f_{rM} for MAT; and CL_{D}/F/f_{rM}/fr_{D} and V_{P,D}/F/f_{rM}/f_{rD} for DAT owing to the administration of an oral dose with uncertain bioavailability (F) and the non-identifiability of the fractions (fᵣ) reflecting conversion of TETA to MAT and MAT to TETA. The additional lag-time (t_{lag}) was used to account for the delay in the up-rising phase of TETA concentration-time profiles observed after oral dosing. Values of half-life (t_{0.5}) were calculated from these parameters.

The pharmacodynamics was modeled assuming a linear relationship between TETA plasma concentrations and urinary excretion of copper (Cho H-Y, Blum R A, Sunderland T, Cooper G J S, and Jusko W J, Pharmacokinetic and pharmacodynamics modeling of a copper-selective chelator (TETA) in healthy adults, J Clin Pharmacol 2009, 49:916-928):

$$Cu ( t ) = {\int }_{{t}_{- 1}}^{t} {ER}_{0} \cdot ( 1 + SL \cdot {C}_{P , T} ( τ ) ) \cdot d τ = {\int }_{{t}_{- 1}}^{t} {ER}_{0} + {ER}_{0} SL \cdot {C}_{P , T} ( τ ) \cdot d τ \qquad ( Equation 9 )$$

where t denotes the urine collection time corresponding to each copper measurement, t−1 is the previous time of bladder voiding (the time range between t₋₁ and t corresponds to the urine collection interval), the d, indicates that the variable of integration is time, ER₀ is the baseline copper excretion rate, and SL is the linear slope value relating copper excretion rate to the plasma TETA concentration.

From Equation (9) the mass of copper excreted (Cu(t)) was integrated over the rate of copper excretion for each urine collection interval. For graphical display ER(t) was approximated as an amount of copper excreted (experimental or model predicted) over the urine collection interval:

$$ER ( t ) = \frac{Cu ( t )}{t - {t}_{- 1}} = \frac{{\int }_{{t}_{- 1}}^{t} {ER}_{0} \cdot ( 1 + SL \cdot {C}_{P , T} ( τ ) ) \cdot d τ}{t - {t}_{- 1}} \qquad ( Equation 10 )$$

Inter-individual variability (IIV) and inter-occasion (IOV) variability for the PK parameters were modeled assuming log normal distribution:

Pᵢₖ=θ_{P}·exp(η_{P,i}+κ_{P,k}) (Equation 12)

where Pᵢₖ is a set of PK/PD parameters for the i^{th} individual and k^{th} occasion, θ_{P} is the population estimate of PK/PD parameters, ηᵢ (ETA) is a random effect with mean 0 and variance ω², κₖ (KAPPA) is an random effect with mean 0 and variance π². A separate model for population variability for F and kₐ was assumed by estimating the inter-occasion variability for those parameters. Two levels of inter-occasion variability were assumed which corresponded to each administration of TETA. For other parameters only inter-individual variability was modeled.

In the analysis, any j^{th} observation of TETA, MAT, and DAT concentration and copper amount in urine for the i^{th} individual on the k^{th} occasion, Cᵢⱼₖ, measured at time tⱼ, was defined by:

$$\begin{matrix}{C}_{P , T , ijk} = {C}_{P , T} ( {P}_{ik} , {t}_{j} ) \cdot ( 1 + {ɛ}_{ijk , T , prop} ) + {ɛ}_{ijk , T , add} & ( Equation 13 ) \\ {C}_{P , M , ijk} = {C}_{P , M} ( {P}_{ik} , {t}_{j} ) \cdot ( 1 + {ɛ}_{ijk , M , prop} ) + {ɛ}_{ijk , M , add} & ( Equation 14 ) \\ {C}_{P , D , ijk} = {C}_{P , D} ( {P}_{ik} , {t}_{j} ) \cdot ( 1 + {ɛ}_{ijk , D , prop} ) + {ɛ}_{ijk , D , add} & ( Equation 15 ) \\ {Cu}_{ijk} = Cu ( {P}_{ik} , {t}_{j} ) \cdot ( 1 + {ɛ}_{ijk , Cu , prop} ) + {ɛ}_{ijk , Cu , add} & ( Equation 16 )\end{matrix}$$

where C_{P,T}, C_{P,M}, C_{P,D}, and Cu reflect the basic structural population model (Eq. 2, 7, 8, 9), Pᵢₖ are pharmacokinetic parameters for the i^{th} individual and k^{th} occasion (i.e. CL_{T}/F, Q/F, V_{P,T}/F, V_{T,T}/F, etc.), and ε_{ikj,add} represents the additive residual intra-individual random errors. It was assumed that εᵢⱼₖ is symmetrically distributed around means of 0, with variance denoted by σ²add and σ²prop for all PK and PD measurements. The NONMEM control stream is below:
$PROBLEM TETA
$INPUT ID TIM AMT DV CMT MDV EVID IDPERIOD IND BLQ AGEy BWkg
Creat DOSE
Form GFR M1F2 Period RACE SEQ TIME
$DATA..\.. \Data\NonmemData20161118.csv IGNORE=#
$SUBROUTINE ADVAN6 TOL=6
$MODEL
COMP=(DEPOT, DEFDOSE);1
COMP=(PER1);2 TAT
COMP=(PER2);3 TAT
COMP=(MET3);4 MAP
COMP=(MET4);5 DAD
COMP=(CU);6 DAD
COMP=(D1);7 D1
COMP=(D2);8 D2
COMP=(D3);9 D3
$PK
; CONVERT PX811019 DOSE TO SYPRINE EQUIVALENTS
CONV=1
IF (FORM.EQ.0) CONV=250/437
DOSECONV=DOSE*CONV; Syprine equivalents
FORX=1;
IF (FORM.EQ.0) FORX=THETA(1); relative bioavailability PX811019/SYPRINE
ALAG1X=THETA(2); ALAG for SYPRINE
IF (FORM.EQ.0) ALAG1X=THETA(3); ALAG for PX811019
KAX=THETA(4); KA for SYPRINE
IF (FORM.EQ.0) KAX=THETA(5); KA for PX811019
; IOV
FVAR1=DEXP(ETA(14)); 1
FVAR2=DEXP(ETA(15)); 2
KAVAR1=DEXP(ETA(16)); 1
KAVAR2=DEXP(ETA(17)); 2
IF (PERIOD.EQ.1) FVAR=FVAR1
IF (PERIOD.EQ.2) FVAR=FVAR2
IF (PERIOD.EQ.1) KAVAR=KAVAR1
IF (PERIOD.EQ.2) KAVAR=KAVAR2
;TETA
ALAG1=ALAGIX*DEXP(ETA(1))
KA=KAX*KAVAR*DEXP(ETA(2))
VT=THETA(6)*DEXP(ETA(3))
CLTM=THETA(7)*DEXP(ETA(4))
VTT=THETA(8)*(1+THETA(21)*(DOSECONV-750))*DEXP(ETA(5))
QT=THETA(9)*DEXP(ETA(6)); MAT
MTT=THETA(10)*DEXP(ETA(7))
VM=THETA(11)*DEXP(ETA(8))
CLMD=THETA(12)*DEXP(ETA(9))
;DAT
VD=THETA(13)*DEXP(ETA(10))
CLD=THETA(14)*DEXP(ETA(11))
;CU
BES=THETA(15)*DEXP(ETA(12))
ALP=THETA(16)*DEXP(ETA(13))
K23=QT/VT
K32=QT/VTT
K50=CLD/VD
K24=CLTM/VT
K45=CLMD/VM
K74=3/MTT
$DES
CTETA=A(2)/VT
DADT(1)=−KA*A(1)
DADT(2)=CONV*FORX*FVAR*KA*A(1)−K23*A(2)+K32*A(3)−K24*A(2)
DADT(3)=K23*A(2)−K32*A(3)
DADT(4)=K74*A(9)*188/146−K45*A(4)
DADT(5)=K45*A(4)*230/188−K50*A(5)
DADT(6)=BES+ALP*CTETA
DADT(7)=K24*A(2)−K74*A(7)
DADT(8)=K74*A(7)−K74*A(8)
DADT(9)=K74*A(8)−K74*A(9)
$ERROR
TETACONC=A(2)/VT
LLOQ_TETA=5/1000
LLOQ_MAT=5/1000 LLOQ_DAT=5/1000
IF (BLQ.EQ.0.AND.CMT.EQ.2) THEN
IPRE=A(2)/VT
IRES=DV-IPRE
W=SQRT(0.00001**2+(THETA(17)*IPRE)**2)
IWRE=(DV-IPRE)/W
Y=IPRE+W*ERR(1)
ENDIF
IF (BLQ.EQ.1.AND.CMT.EQ.2) THEN
IPRE=A(2)/VT
IRES=DV-IPRE
W=SQRT(0.00001**2+(THETA(17)*IPRE)**2)
DUM=(LLOQ_TETA-IPRE)/W
CUMD=PHI(DUM) F_FLAG=1
Y=CUMD
ENDIF
IF (BLQ.EQ.0.AND.CMT.EQ.4) THEN
IPRE=A(4)/VM
IRES=DV-IPRE
W=SQRT(0.00001**2+(THETA(18)*IPRE)**2)
IWRE=(DV-IPRE)/W
Y=IPRE+W*ERR(2)
ENDIF
IF (BLQ.EQ.1.AND.CMT.EQ.4) THEN
IPRE=A(4)/VM
IRES=DV-IPRE
W=SQRT(0.00001**2+(THETA(18)*IPRE)**2) DUM=(LLOQ_MAT-IPRE)/W
CUMD=PHI(DUM)
F_FLAG=1
Y=CUMD
ENDIF
IF (BLQ.EQ.O.AND.CMT.EQ.5) THEN
IPRE=A(5)/VD
IRES=DV-IPRE
W=SQRT(0.00001**2+(THETA(19)*IPRE)**2)
IWRE=(DV-IPRE)/W
Y=IPRE+W*ERR(3)
ENDIF
IF (BLQ.EQ.1.AND.CMT.EQ.5) THEN
IPRE=A(5)/VD
IRES=DV-IPRE
W=SQRT(0.00001**2+(THETA(19)*IPRE)**2)
DUM=(LLOQ_DAT-IPRE)/W
CUMD=PHI(DUM) F_FLAG=1
Y=CUMD
ENDIF
IF (CMT.EQ.6) THEN
IPRE=A(6)
IRES=DV-IPRE W=SQRT(0.00001**2+(THETA(20)*IPRE)**2)
IWRE=(DV-IPRE)/W
Y=IPRE+W*ERR(4)
ENDIF
;TETA
$THETA (0,0.746); F_RELATIVE
$THETA (0,0.083); ALAG1_SYPRINE
$THETA (0,0.100); ALAG1_PX811019
$THETA (0,1.72); KA_SYPRINE
$THETA (0,1.20); KA_PX811019
$THETA (0,376.); VT
$THETA (0,147.); CLTM
$THETA (0,1160.); VTT
$THETA (0,42.1); QT
; MAT
$THETA (0,0.549); MTT
$THETA (0,395.); VM
$THETA (0,75.8); CLMD
;DAT
$THETA (0,179.); VD
$THETA (0,306.); CLD; CU
$THETA (0,0.592); ER0
$THETA (0,22.9); ER0*SL
$THETA (0.001); VT-DOSE
; ERROR MODELS
$THETA (0,0.456); PROPT
$THETA (0,0.227); PROPM
$THETA (0,0.196); PROPD
$THETA (0,0.542); PROPCU
$OMEGA 0 FIX; ALAG1
$OMEGA 0 FIX; KA $OMEGA 0.0141; VT
$OMEGA 0.0213; CLTM
$OMEGA 0.0626; VTT $OMEGA 0 FIX; QT
$OMEGA 0 FIX; MTT
$OMEGA BLOCK(2); VM-CLMD
0.25
0.1 0.157
$OMEGA BLOCK(2); VD-CLD
0.694
0.1 0.127
$OMEGA 1.26; ER0
$OMEGA 0.282; ER0*SL
$OMEGA BLOCK(1) 0.20;FVAR
$OMEGA BLOCK(1) SAME
$OMEGA BLOCK(1) 0.60;KAOCC
$OMEGA BLOCK(1) SAME
$SIGMA 1. FIX;FIX
$SIGMA 1. FIX;FIX
$SIGMA 1. FIX;FIX
$SIGMA 1. FIX;FIX
$ESTIMATION METHOD=COND INTER NOABORT MAXEVAL=9999 NSIG=2
SIGL=7 PRINT=2 LAPLACIAN NUMERICAL SLOW
MSFO=
$COV UNCONDITIONAL SLOW
$TABLE ID TIME EVID IPRE IWRE IRES AMT BLQ CWRES CMT TIM MDV
NOPRINT ONEHEADER FILE=SDTAB.BLE
$TABLE ID ALAG1 KA VT VTT QT VM VD CLD BES MTT FVAR KAVAR ALP
CLTM CLMD ETA1 ETA2 ETA3 ETA4 ETA5 ETA6 ETA7 ETA8 ETA9 ETA10
ETA11 ETA12
ETA13
NOAPPEND NOPRINT ONEHEADER FILE=PATAB.BLE
$TABLE ID AGEy BWkg Creat DOSE DOSECONV GFR
NOAPPEND NOPRINT ONEHEADER FILE=COTAB.BLE
$TABLE ID M1F2 Period RACE SEQ Form
NOAPPEND NOPRINT ONEHEADER FILE=CATAB.BLE

Visual Predictive Checks: The model performance was assessed by means of Visual Predictive Checks (VPC). The VPC was calculated based on 1000 datasets simulated with the final parameter estimates [7-9]. The VPC enables the comparison of predicted versus observed data over time. In this study the 10th, 50th and 90th percentiles were used to summarize the data and VPC prediction. The VPC enables the comparison of the confidence intervals obtained from prediction with the observed data over time. When the corresponding percentile from the observed data falls outside the 90% confidence interval derived from predictions, it is an indication of a model misspecification.

Covariance Analysis: One purpose of this work was to characterize possible PK/PD differences for TETA given as PX811019 versus Syprine®. Thus, all absorption-related parameters (lag-time and ka) were estimated separately for each formulation. Other parameters were assumed to be identical between drug formulations, unless some contrary evidence was found during the model building process.

Other possible relationships were sought using a standard covariance analysis where individual (post-hoc) estimates of the PK/PD parameters (Eta (η) or Kappa (κ)) were plotted against available covariates (weight, age, eGFR, sex, dose, sequence, period, formulation) to identify their potential effects. If the relationship was found, all the recorded values were described by means of the following regression model:

Pᵢₖ=θ_{P1}(1+θ_{P2}(COVᵢₖ−COV_{median}))exp(η_{P,i}+κ_{P,k}) (Equation 17)

where the θ_{P1} and θ_{P2} are the regression coefficients. Continuous variables were centered around their median values, COVmedian, thus allowing θ_{P1} to represent the parameter estimate for the typical patient with median covariates. Categorical covariates (such as sex) were included in the model based on indicator variables:

$${P}_{ik} = ( \begin{matrix}{θ}_{P 1} if {IND}_{ik} = 0 \\ {θ}_{P 2} if {IND}_{ik} = 1\end{matrix} ) \cdot exp ( {η}_{P , i} + {κ}_{P , k} ) \qquad ( Equation 18 )$$

where IND is an indicator variable that has a value of 1 when the covariate is present and 0 otherwise. The difference in the minimum of the NONMEM objective function (OFV) obtained for the two hierarchical models (likelihood ratio) is approximately χ2-distributed (Mould DR, Upton RN. Basic concepts in population modeling, simulation, and model-based drug development-Part 2: Introduction to pharmacokinetic modeling methods, CPT: Pharmacometrics & Systems Pharmacology 2013, 2, e38). During the covariate search the effect of each covariate was examined by adding an appropriate equation to the base model. When the difference in OFV between the models amounted to 3.84 for one degree of freedom, it was considered to be statistically significant (at p<0.05) for the covariate to be included into the base model. This process was repeated until all significant covariates were added. Then backward elimination was performed by removing one covariate at a time. The least important covariate was dropped out from the model according to the OFV unless that difference in OFV was larger than 6.63 (corresponding to p<0.01). The final model was established when no more covariates could be excluded from the model.

### Results and Discussion

The data analyzed from the 18 subjects contained 714 plasma concentration measurements for each of TETA, MAT and DAT, and 455 copper measurements in urine. There were 124 (17.4%) TETA, 113 (15.8%) MAT, and 187 (26.2%) DAT measurements that fell below the quantification limit (BQL).

Table 7 presents a summary of the subject characteristics and the available covariates.

TABLE 7
Demographic characteristics of subjects.

| | All Subjects, | |
|---|---|---|
| Parameter, | Median [Range] | |
| units | or Number | |
| Age, yr | 34 | [20-48] |
| Weight, kg | 75 | [57.3-93.6] |
| Glomerular Filtration Rate (eGFR), ml/min | 121.5 | [91.3-158.8] |

| | |
|---|---|
| Male/Female | 9/9 |

The median age of the group of 9 males and 9 females was 34 years with a range of 20 and 48 years. The body weights ranged from 57.3 to 93.6 kg. All subjects had normal kidney function with the estimated glomerular filtration rate (eGFR) within a range of 91.3 to 158.8 ml/min.

Table 6 in Example 13 provides a summary of the major exposure indices of TETA, MAT, and DAT using traditional noncompartmental (NCA) analysis. It is evident from the Cₘₐₓ and AUC values that these equimolar doses of TETA produce lower concentrations of all three compounds when administered as PX811019. However, as the NCA does not account appropriately for later time BLQ values, any parameters dependent on such (e.g. t0.5) may be skewed. As this study included a range of doses and joint measurements of the parent drug, two metabolites, and copper excretion, this population-based analysis was enacted to compare the two salt forms in this global, more generalized fashion.

Initially, a PK/PD model was used to describe data from a multiple-dosing study in healthy volunteers. It is a two-compartment disposition model with first-order absorption for TETA PK. The metabolites of TETA were modeled assuming catenary metabolism (TETA→MAT→DAT). Additionally, three transit steps were used to model the delay between TETA and MAT concentrations. A one-compartment disposition model was assumed for both MAT and DAT plasma concentrations. The copper in urine was modeled as a direct linear connection to TETA plasma concentrations as found earlier. Cho, H-Y, et al., Pharmacokinetic and pharmacodynamics modeling of a copper-selective chelator (TETA) in healthy adults, J Clin Pharmacol 2009, 49:916-928.

The following modifications to the Cho, H-Y, et al., model were applied: (i) an additive part of the residual error model was not needed; (ii) estimation of inter-individual variability for ER₀ (baseline copper excretion rate) was included owing to greater variability in this study; (iii) re-parametrization of Equation (9) to ER₀ and SL·ER₀ as independent parameters; (iv) inclusion of correlations between apparent volume of distribution and clearance for MAT and DAT; (v) modelling the IOV for the absorption rate constant as part of the key purpose of this study; and (vi) dose-dependence of TETA peripheral volume of distribution was found. Each of those steps improved the model fitting considerably as judged by the NONMEM objective function and visual predictive checks.

The experimental data and model fittings for plasma concentrations of TETA, MAT, and DAT and copper excretion over the entire study were graphed for each of the subjects (Subject Graphs).

Modeling inter-occasion variability in presumed general bioavailability (F) and absorption rate constant was used as a surrogate to account for overall intra-subject variation in TETA pharmacokinetics. Such inclusion of inter-occasion variability avoids bias in the population parameter estimates. Bergstrand, M, et al., Prediction-corrected visual predictive checks for diagnosing nonlinear mixed-effects models AAPS J. 2001, 13: 143-151.

The model fittings in the Subject Graphs showed that the final PK model described the measured concentrations and PD responses accurately. The typical goodness-of-fit diagnostic plots for the final model were prepared. The individual and population predictions versus observed concentrations are relatively symmetrically distributed around the line of identity, the individual weighted residuals versus individual predicted concentrations and versus time do not show any trend and are relatively uniformly distributed around zero indicating good model performance in quantifying the PK data.

The VPC plots for the TETA, MAT, and DAT concentrations and copper amounts excreted in urine were used to assess the properties of the model and fitted parameters. The VPC plots indicated that both the central tendency of the data and the variability at a particular sampling time were recaptured well as most of the data points fall within the 90% Confidence Intervals. There were no major misspecifications in the model fittings with respect to the measurements and fractions of concentrations falling below the LLOQ.

The model-fitted population PK/PD parameters for TETA and metabolites are listed in Tables 8A-8D, below:

TABLE 8
Summary of the final population PK/PD parameters (A) along with inter-subject (B), inter-
occasion (C), and residual error variance estimates (D) based on the final model.

| | | |
|---|---|---|
| Parameter, | | Estimates (% CV) |
| Units | Description | [Shrinkage] |

A. Population means

| | | |
|---|---|---|
| θ - FP/S | Relative bioavailability (PX811019/Syprine) | 0.745 (6.0) |
| θ - t_{lag}, h (Syprine) | Absorption lag time for Syprine | 0.083 (0.3) |
| θ - t_{lag}, h (PX811019) | Absorption lag time for PX811019 | 0.239 (1.4) |
| θ - kₐ, h⁻¹ (Syprine) | Absorption rate constant for Syprine | 1.74 (55.1) |
| θ - kₐ, h⁻¹ (PX811019) | Absorption rate constant for PX811019 | 1.19 (31.1) |
| θ - V_{P, T}/F, L | Apparent central volume for TETA | 326 (15.0) |
| θ - CL_{T}/F, L/h | Apparent systemic clearance for TETA | 141 (11.1) |
| θ - Q_{T}/F, L/h | Apparent distribution clearance for TETA | 39.2 (12.1) |
| θ - V_{T, T}/F, L | Apparent peripheral volume for TETA for 750 | 1210 (11.8) |
| | mg dose (Syprine equivalents) | |
| θ - (V_{T, T}/F-Dose), L/100 mg | Regression parameters in relationship between | 0.0637 (17.3) |
| | the TETA dose (Syprine equivalents) and V_{T, T}/F | |
| θ - MMT, h | Mean transit time for MAT formation | 0.381 (34.2) |
| θ - V_{P, M}/F/frM, L | Apparent volume for MAT | 426 (9.8) |
| θ - CL_{M}/F/fr_{M}, L/h | Apparent systemic clearance for MAT | 76.2 (6.5) |
| θ - V_{P, D}/F/frM/frD, L | Apparent volume for DAT | 197 (17.7) |
| θ - CL_{D}/F/fr_{M}/fr_{D}, L/h | Apparent systemic clearance for DAT | 306 (9.0) |
| θ - ER₀, μg/h | Baseline copper excretion rate | 0.581 (28.7) |
| θ - SL · ER₀, (mg/L)⁻¹ · (μg/h) | Slope between TETA and Cu excretion rate | 24.3 (17.4) |

A. Inter-individual Variability

| | | |
|---|---|---|
| ω² - V_{P, T}/F, % | Inter-individual variability of V_{P, T}/F | 16.3 (27.9) [22.3] |
| ω²- CL_{T}/F, % | Inter-individual variability of CL_{T}/F | 10.3 (41.4) [22.0] |
| ω² - V_{T, T}/F, % | Inter-individual variability of V_{T, T}/F | 13.5 (33.6) [30.9] |
| ω² - V_{P, M}/F/frM, % | Inter-individual variability of V_{P, M}/F/f_{M} | 55.1 (17.4) [2.7] |
| ω²- CL_{M}/F/fr_{M}, % | Inter-individual variability of CL_{M}/F/f_{M} | 41.5 (15.1) [2.9] |
| ω² - CL_{D}/F/fr_{M}/fr_{D}, % | Inter-individual variability of CL_{D}/F/f_{M}/f_{D} | 55.9 (18.1) [1.5] |
| ω² - V_{P, D}/F/frM/frD, % | Inter-individual variability of V_{P, D}/F/f_{M}/f_{D} | 86.8 (29.4) [2.7] |
| ω²- ER₀, % | Inter-individual variability of ER₀ | 112 (24.6) [5.9] |
| ω²- SL · ER₀, % | Inter-individual variability of SL · ER₀ | 58.1 (25.1) [7.8] |
| Cor₁ | Correlation between V_{P, M}/F/fr_{M} and CL_{M}/F/frₘ) | 0.927 |
| Cor₂ | Correlation between V_{P, D}/F/fr_{M}/fr_{D} and | 0.789 |
| | CLD/F/frM/frD) | |

B. Inter-Occasion Variability

| | | |
|---|---|---|
| π²-kₐ, % | Inter-occasion variability of kₐ | 69.4 (57.6) [16.3] |
| π²-F, % | Inter-occasion variability of F | 43.7 (17.3) [5.5] |

C. Residual variability

| | | |
|---|---|---|
| σ²prop, T, % | Proportional residual error variability for TETA | 38.1 (7.5) [2.6] |
| σ²prop, D, % | Proportional residual error variability for MAT | 23.8 (7.3) [4.9] |
| σ²prop, M, % | Proportional residual error variability for DAT | 19.0 (4.0) [4.7] |
| σ²prop, Cu, % | Proportional residual error variability for Cu | 56.6 (5.4) [1.8] |

All of the PK/PD parameters, inter-subject, inter-occasion and residual error variances were estimated well with CV values smaller than 57.6%. The apparent mean central volume of distribution was 326 L for TETA, 426 L for MAT, and 197 L for DAT. The corresponding apparent clearances were 141, 76.2, and 306 L/h. For TETA the apparent peripheral volume was 1210 L for the 750 mg dose and the apparent distribution clearance was 39.2 L/h. The relative bioavailability of PX811019 versus Syprine® was 74.5%. The absorption rate constant was 1.19 h⁻¹ for PX811019 and 1.74 h⁻¹ for Syprine® and time-lags were 0.239 and 0.083 h. The mean transit time (MTT) relating TETA conversion to MAT was 0.381 h reflecting a brief delay in appearance of MAT. The baseline copper excretion rate was 0.581 μg/h. Copper excretion increased linearly in relation to TETA concentrations with a slope (SL) of 41.8 (mg/L)⁻¹.

The inter-individual variability (IIV) was generally modest to moderate and could be identified for all of the central volumes of distribution (16.3%, 55.1%, and 86.8% for TETA, MAT and DAT), all apparent systemic clearances (10.3%, 41.5% and 55.9% for TETA, MAT and DAT), for the volume of peripheral compartment for TETA (13.5%), for ER₀ (112%) and for the SL·ER₀ (58.1%). For other parameters the IIV was fixed to zero as it either tended to zero during the model-building process or was estimated with a large (>50%) shrinkage.

The repeated administration of drug leads to the occurrence of IOV. This process, when included during the model building process, substantially improved model fittings. The IOV for the presumed F and absorption rate constant was moderate and equal to 44% and 70%.

A significant relationship (p=9.0224e-05, Δ OFV=15.331) was found between V_{T,T}/F and TETA dose expressed in Syprine equivalents. The model predicted V_{T,T}/F increased by 6.37% for every 100 mg difference from the 750 mg dose of TETA. This factor may be responsible for the modest increase in half-life with dose (Table 6 in Example 13). In the final model individual values of parameters with IIV were estimated precisely as indicated by very low shrinkage of less than 20% (except V_{T,T}/F for which shrinkage equaled 31%). Savic, R M and Karlsson, M O. The importance of shrinkage in empirical Bayes estimates for diagnostics: problems and solutions. AAPS J. 2009; 11: 558-69. This suggests that the data are informative about the individual-predicted parameters making possible the search for other covariate relationships. Relationships between the factors of weight, age, eGFR, sex, and sequence were sought based on the ETA plots (deviation of the individual estimate from the population mean) using the individual estimates for ETA of TETA PK/PD parameters in relation to the sex of the subjects. Similarly, relationships between the factors of formulation and occasion were sought based on KAPPA plots (deviation of the estimate at a particular occasion (visit) from the individual mean PK parameter) of TETA PK/PD parameters in relation to formulation and occasion. The lack of any trend in these data indicates that these individual covariates do not account for the remaining unexplained inter-subject variability in the PK/PD parameters.

Individual TETA, MAT, and DAT concentrations and copper excretion rates versus time were also graphed jointly for 6 typical subjects. The drug and metabolite concentrations over the full study period as well as copper excretion rates appear similar and consistent for all subjects.

The model-fitted half-life values were added to Table 6 in Example 13 for comparison with the NCA values. The beta t_{0.5} for TETA increased with dose from 18 to 28 to 37 hours due to the increase in V_{T} with dose. The LLOQ of the assay was improved for this study allowing for more extended and reliable measurements during the washout phase. An increase in VT such as this is usually explained by either nonlinear plasma protein binding or increased tissue binding with drug concentration. The listed t_{0.5} values for DAT and MAT in Table 6 (Example 13) reflect theoretical disposition rates that would be expected if these compounds were administered directly. Their actual terminal slopes are governed by “formation rate-limited disposition” from TETA and are determined from such in the process of joint fitting of the entirety of the data.

The PK/PD model applied to copper excretion showed a highly consistent relationship between TETA concentrations and copper excretion that superimpose for Syprine® and PX811019 for each subject across all doses. One subject had unusually high baseline and TETA-affected copper excretion rates.

Overall, the administration of TETA as the succinate salt produces generally linear properties and PK/PD profiles that are indistinguishable from TETA given as the dihydrochloride except for lower general exposures reflected as 74.5% relative bioavailability. The absorption kinetics of the two forms differ, but only slightly. The lower Cₘₐₓ and AUC values observed in preliminary analysis of these data (Example 13, Table 6) with PX811019 can be compensated for by administration of amounts of 134% of the present succinate formulation (1/0.745). The concentrations versus time 8798 of TETA that can expected after such triethylenetetramine disuccinate dose adjustments should be the same as triethylenetetramine dihydrochloride profiles.

### Example 15

### Evaluation of Pgp Involvement in Compound Permeability Through the Use of Caco-2 In Vitro Model for Oral Bioavailability

The presence of p-glycoprotein (Pgp) efflux pumps in mammalian intestine tissue have been previously demonstrated and play a key role in the active transport mechanisms of drugs.

The aim of this study was to evaluate the implication of Pgp in the permeability and metabolism of a test compound, the triethylenetetramine disuccinate (PX811019), using the Caco-2 in vitro model for the human intestinal barrier.

As a highly soluble and low toxicity compound in vitro, and based upon the in vitro Papp value described below, it is predicted that triethylenetetramine disuccinate will have good absorption in humans (estimated at approximately 70%).

The most commonly used models in intestinal transport studies are human intestinal cell lines, specifically the HT29 and Caco-2 cell lines, derived from colon carcinoma (Wils P., et al. Differentiated intestinal epithelial cell lines as in vitro models for predicting the intestinal absorption of drugs. Cell Biol. Toxicol. 10:393, 1994; Boulenc X. Intestinal Cell Models: Their use in evaluating the metabolism and absorption of xenobiotics. STP. Pharma. Sciences 7:259, 1997), and the most widely used in pharmaceutical research to evaluate intestinal absorption are the Caco-2 cells. Meunier V, et al. The human intestinal epithelial cell line Caco-2; Pharmacological and pharmacokinetic applications. Cell Biol. Toxicol. 11:187, 1995. Cultured on a solid permeable membrane for 21 days under conditions that enhance their polarization, this colon carcinoma-derived cell line achieves a confluent monolayer of fully differentiated cells, with apical microvilli mimicking the intestinal lumen and a completely differentiated basolateral surface, equivalent to the cell surface normally in contact with the blood system. Furthermore, they present phenotypic and physiological characteristics that closely resemble enterocytes from the human small intestine epithelium. Zweibaum A., et al. Use of cultured cell lines in studies of intestinal cell differentiation and function. In: M. Field and R. A. Frizzel (eds), Handbook of physiology. The gastrointestinal system. Vol IV: Intestinal absorption and secretion. American Physiological Society, Washington D.C. 223, 1991. See Artursson P., and Karlsson J. Correlation between oral drug absorption in humans and apparent drug permeability coefficients in human intestinal epithelial (Caco-2) cells. Biochem. Biophy. Res. Com. 175:880, 1991.

The principal objective of this project was to address the Pgp involvement in metabolism and permeability of a [¹⁴C]-radiolabeled test substance, triethylenetetramine disuccinate ([2-¹⁴C]PX811019), and to further determine if unlabeled triethylenetetramine disuccinate test substance (PX811019) represents a Pgp inhibitor or substrate. To this aim, polarized cultures of Caco-2 cells which have been assessed for monolayer integrity and functionality were used as an in vitro model for the GI barrier.

Evaluation of unlabeled triethylenetetramine disuccinate (PX811019) cytotoxicity on Caco-2 cultures: To this aim, a WST-1 assay was performed. This standard assay for measuring cell proliferation, cell viability and cytotoxicity in mammalian cells utilizes measurement of mitochondrial succinate dehydrogenase activity as an index of mitochondrial damage, and is accepted as one of the most sensitive to detect early cytotoxicity events. Caco-2 cells were seeded in 96-well plates, at a density of 5×10⁵cells/cm², such as in permeability assays. After 48 hours of culture, unlabeled PX811019 was applied at 8 different concentrations (1, 0.5, 0.25, 0.125, 0.0625, 0.0312, 0.0156, 0.0078 mM) in HBSS(×1)-Ca²⁺Mg²⁺-pH=7.4 buffer, and was incubated for 2 hours at 37° C. Cells were then checked for viability through WST-1 application and measurement of absorbance at 450 nm in an ELISA plate reader. Each concentration was tested in triplicate.

Evaluation of [2-¹⁴C] triethylenetetramine disuccinate (PX811019) permeability in Caco-2 barrier model: Once cytotoxicity to Caco-2 cultures had been assessed, [2-¹⁴C]PX811019 was incubated on 21-day Caco-2 polarized cultures in transwell filters (6.5 mm diameter; 0.4 μm pore). TEER measurement and permeability of Lucifer yellow (low permeability marker) and Antipyrin (high permeability marker) were first performed to check for barrier integrity and quality. Digoxin was used as marker to check for Pgp activity in the cultures. In parallel, the effect of test compound on barrier integrity was determined by applying unlabelled PX811019, at the concentration used in the permeability assay, together with Lucifer yellow in apical compartments of control transwell filters. For permeability assessment, [2-¹⁴C]PX811019 was then applied in donor compartments at one nontoxic concentration (1 μCi/ml; 0.02 mM) in HBSS(×1)-Ca²⁺Mg²⁺-pH=7.4 buffer, and incubated for 1 hour at 37° C., alone or in the presence of verapamil or sodium azide. Samples were recovered from receptor compartments after 0, 15, 30, 45, 60, and 120 minutes, and further analyzed by liquid scintillation counting. Samples were also recovered a time 0 and 120 minutes from the donor compartments for mass balance evaluation. Each condition was performed in 3 replicated transwell filters in the presence of the Caco-2 barrier. Based on dpm primary data obtained from sample analysis by scintillation counting, permeability coefficient (Papp in cm/s) was calculated in both A-B (apical-basal) and B-A (basal-apical) directions, and [2-¹⁴C]PX811019 permeability was evaluated under each experimental condition.

Evaluation of unlabeled triethylenetetramine disuccinate (PX811019) effect on Pgp activity in Caco-2 barrier model: Once evaluated for its permeability on Caco-2 cultures, [³H]-digoxin was incubated alone or together with unlabeled triethylenetetramine disuccinate (PX811019) on 21-day Caco-2 polarized cultures in transwell filters (6.5 mm diameter; 0.4 μm pore). TEER measurement and permeability of Lucifer yellow (low permeability marker) and Antipyrin (high permeability marker) were first performed to check for barrier integrity and quality. In parallel, the effect of test compound on barrier integrity was determined by applying unlabeled PX811019, at the concentration used in the permeability assay, together with Lucifer yellow into apical compartments of control transwell filters. To assess the effect of test compound on Pgp activity, [³H] digoxin (4 μCi/ml; 0.2 mM) was then applied alone or in the presence of a similar and nontoxic concentration of PX811019 test compound (0.2 mM) into donor compartments in HBSS(×1)-Ca²⁺Mg²⁺-pH=7.4 buffer, and incubated for 1 hour at 37° C. Samples were recovered from receptor compartments after 0, 15, 30, 45, 60, and 120 minutes, and further analyzed by liquid scintillation counting. Samples were also recovered at time 0 and 120 minutes from the donor compartments for mass balance evaluation. Each condition was performed in 3 replicated transwell filters with in the presence of the Caco-2 barrier. Based on dpm primary data obtained from sample analysis by scintillation counting, the permeability coefficient of [³H]digoxin (Papp in cm/s) was calculated in both A-B (apical-basal) and B-A (basal-apical) directions, and the effect of PX811019 on Pgp-dependent digoxin permeability was evaluated under each experimental condition.

### Main Results

Unlabeled triethylenetetramine__disuccinate (PX811019) did not present any cytotoxicity on Caco-2 cells at any of the concentrations tested.
Caco-2 polarized monolayers used in this study fulfilled the quality criteria for barrier status required for predictive in vitro permeability assay: TEER values were higher than 1000 ohm·cm²; Papp values for Lucifer yellow (low permeability marker) were lower than 1×10⁻⁶ cm/s and Papp values for Antipyrin (high permeability marker) were higher than 1×10⁻⁶ cm/s in both experiments.
Digoxin presented a low Papp value in the A-B direction (0.75±×10⁻⁶ cm/s) and medium Papp value in the B-A direction (6.08×10⁻⁶ cm/s), with an Asymmetry Index of 8.06, indicative of Pgp activity in the system. In contrast, [2-¹⁴C] triethylenetetramine_disuccinate (PX811019) applied on these Caco-2 monolayers presented medium-high A-B permeability values at the nontoxic concentration tested, with a mean value of 9.87×10⁻⁶ cm/s, and no permeability was observed either in the B-A direction, or in presence of verapamil or sodium azide. Mass balance was between 70 and 120% for all the conditions tested.
In the presence of the test compound, triethylenetetramine disuccinate (PX811019), Papp values of Digoxin in either A-B or B-A directions were similar to those obtained with Digoxin alone when test compound was applied apically (0.66±0.89×10⁻⁶ cm/s and 12.7±6.76×10⁻⁶ cm/s, respectively), and both were slightly reduced when applied basolaterally (0.2±0.35×10⁻⁶ cm/s and 5.68±3.03×10⁻⁶ cm/s, respectively). However, in both situations, the Asymmetry Index was maintained and even increased. This indicates that the Digoxin transport pathway, and thus Pgp activity, were not affected by the application of the unlabeled test substance triethylenetetramine disuccinate (PX811019), independently of the compartment where it was applied.

In conclusion, triethylenetetramine disuccinate (PX811019) did not exhibit any cytotoxicity on Caco-2 cells at any of the concentrations tested (1, 0.5, 0.25, 0.125, 0.0625, 0.0312, 0.0156, 0.0078 mM). Caco-2 polarized monolayers used in this study fulfilled the quality criteria for barrier status required for predictive in vitro permeability assay: TEER values were higher than 1000 ohm·cm²; Papp Lucifer yellow was lower than 1×10⁻⁶ cm/s and Papp Antipyrin higher than 10×10⁻⁶ cm/s. Furthermore, Papp values and Asymmetry Index obtained for Digoxin indicated that levels of Pgp activity were within an acceptable range for this cell model.

Additionally, triethylenetetramine disuccinate (PX811019) did not affect the integrity of the monolayer at the concentrations used in both assays. Trientine disuccinate (PX811019) applied on these Caco-2 monolayers presented medium-high permeability values at the concentration tested, with a mean value of 9.87×10⁻⁶ cm/s, in the absorptive (A-B) direction. As a highly soluble and low toxicity compound in vitro, and based upon the in vitro Papp value, one can predict that this compound will have good absorption in humans (estimated at approximately 70%). No permeability was observed in the secretory (B-A) direction.

Comparing with permeability data from Digoxin Pgp substrate, Papp data obtained in both A-B and B-A directions suggested that triethylenetetramine disuccinate (PX811019) crosses the Caco-2 barrier using a Pgp-independent polarized transport pathway in the absorptive direction. Triethylenetetramine disuccinate (PX811019) A-B transport through Caco-2 monolayer was fully inhibited in the presence of sodium azide and verapamil. As sodium azide is an ATP synthesis inhibitor, this suggested that test substance (PX811019) transport was ATP-dependent. Verapamil is usually used in the permeability assay as a Pgp inhibitor through inhibition of the ATP binding cassette. However, it has been extensively described as a Ca²⁺ channel blocker, more specifically L-type channels. As the A-B polarization of triethylenetetramine disuccinate transport and Asymmetry Index indicated that Pgp was not implicated, the data in the presence of verapamil would then suggest that this agent acts as a blocker of a specific transporter for triethylenetetramine disuccinate. On the basis of these data, a possible mechanism of transport through intestinal barrier of test substance could be through an active ATP-dependent and/or Ca²⁺-dependent transporter pathway.

The data obtained from the permeability assay with Digoxin alone and in presence of test substance showed that triethylenetetramine disuccinate did not affect Pgp activity. Furthermore, as it did not compete with Digoxin, it would not be a Pgp substrate, which further supports the data showing that its permeability is not Pgp-dependent in the cell model and experimental conditions used in the study.

The inventions described and claimed herein have many attributes and embodiments including, but not limited to, those set forth or described or referenced in this Detailed Disclosure. It is not intended to be all-inclusive and the inventions described and claimed herein are not limited to or by the features or embodiments identified in this Detailed Disclosure, which is included for purposes of illustration only and not restriction. A person having ordinary skill in the art will readily recognize that many of the components and parameters may be varied or modified to a certain extent or substituted for known equivalents without departing from the scope of the invention. It should be appreciated that such modifications and equivalents are herein incorporated as if individually set forth. The invention also includes all of the steps, features, compositions and compounds referred to or indicated in this specification, individually or collectively, and any and all combinations of any two or more of said steps or features.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents. Reference to any applications, patents and publications in this specification is not, and should not be taken as, an acknowledgment or any form of suggestion that they constitute valid prior art or form part of the common general knowledge in any country in the world.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, and in embodiments or examples of the present invention, any of the terms “comprising”, “consisting essentially of”, and “consisting of” may be replaced with either of the other two terms in the specification. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants. Furthermore, titles, headings, or the like are provided to enhance the reader's comprehension of this document, and should not be read as limiting the scope of the present invention. Any examples of aspects, embodiments or components of the invention referred to herein are to be considered non-limiting.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

We claim:

1. A method of treating coronavirus disease in a subject caused by exposure to a copper-requiring coronavirus, the method comprising administering to the subject a composition comprising an effective amount of a copper-depriving agent, wherein one or more symptoms of the disease are reduced.

2. The method of claim 1, wherein the coronavirus infection is caused by a SARS-CoV-2 coronavirus.

3. The method of claim 1, wherein the coronavirus disease is COVID-19 disease.

4. The method of claim 1, wherein the copper depriving agent lowers or reduced copper values content, intracellular copper and/or total copper in the subject.

5. The method of claim 1, wherein the copper depriving agent inhibits copper transport into a copper-requiring coronavirus host cell.

6. The method of claim 1, wherein the copper depriving agent is a copper chelating compound that preferentially binds Cu¹⁺ or that preferentially binds Cu²⁺ or that binds both.

7. The method of claim 6, wherein the chelator is selected from the group consisting of triethylenetetramine, ammonium tetrathiomolybdate, D-penicillamine and N-acetylpenicillamine.

8. The method of claim 7, wherein the triethylenetetramine is triethylenetetramine dihydrochloride, triethylenetetramine tetrahydrochloride, triethylenetetramine disuccinate, triethylenetetramine disuccinate anhydrate and/or a triethylenetetramine disuccinate polymorph.

9. The method of claim 8, wherein the triethylenetetramine is formulated in a capsule for oral administration.

10. The method of claim 1, wherein the composition is formulated for nasal, intrasinal, intrapulmonary and/or endosinusial administration.

11. The method of claim 1, wherein the copper-depriving agent is a copper chelator and is administered in an amount ranging from about 600 to about 2400 milligrams per day.

12. A method of preventing or treating coronavirus infection in a subject caused by exposure to a copper-requiring coronavirus, the method comprising administering to the subject, either before or after the exposure, a composition comprising an effective amount of a copper-depriving agent that lowers copper available to a coronavirus values by removing copper from or reducing intracellular copper in the subject, wherein the method results in reducing infectious coronavirus organisms and/or coronavirus particles and preventing infection or reducing the infection in the subject.

13. A method of preventing or reducing coronavirus infection in a subject caused by exposure to a COVID-19 coronavirus, the method comprising administering to the subject, either before or after the exposure, a composition comprising an effective amount of a copper-depriving compound selected from the group consisting of triethylenetetramine, ammonium tetrathiomolybdate, D-penicillamine and N-acetylpenicillamine, wherein the method results in reducing infectious coronavirus organisms and/or coronavirus particles and preventing infection or reducing the infection in the subject.

14. The method of claim 1, wherein: (a) the coronavirus comprises human coronavirus 229E, human coronavirus OC43, SARS-CoV, a HCoV-NL63, HKU1, MERS-CoV, or SARS-CoV-2; and/or (b) the risk of infection to be prevented or reduced is by coronavirus disease 2019 (COVID-19); and/or (c) the coronavirus comprises (i) a polynucleotide comprising SARS-CoV-2 (GenBank accession number NC_0455122), or (ii) a copper-requiring strain or mutation thereof, or (iii) an infectious fragment thereof coding for or included within a viable or infectious viral particle susceptible to copper deprivation, or (iv) a copper-requiring infectious polynucleotide having at least 80% sequence identity to the polynucleotide comprising SARS-CoV-2.

15. The method of claim 1, wherein the administering comprises administration of a nasal spray, medicated nasal swab, medicated wipe or aerosol comprising the composition to the subject's nasal vestibule or nasal passages.

16. The method of claim 1, wherein the subject is a healthcare worker, elderly person, frequent traveler, military personnel, caregiver, within the BAME group, or a subject with a preexisting condition that results in increased risk of mortality with infection, and optionally wherein the preexisting condition comprises cancer, chronic kidney disease, chronic obstructive pulmonary disease, organ transplant, sickle cell disease, diabetes, type 2 diabetes, type 1 diabetes, hypertension, obesity, pulmonary fibrosis, heart disease or an immunocompromised state.

17. The method of claim 1, wherein the administering further comprises administration of one or more antiviral drugs selected from the group consisting of chloroquine, hydroxychloroquine, darunavir, galidesivir, an interferon, lopinavir, ritonavir, remdesivir, and triazavirin.

18. The method of claim 18, wherein the interferon is selected from the group consisting of interferon β-1b, pegylated interferon β-1b, interferon α-n1, pegylated interferon α-n1, interferon α-n3, pegylated interferon α-n3 and human leukocyte interferon α.

19. The method of claim 1, wherein the composition further comprises a therapeutic agent, wherein the therapeutic agent is: (a) an antimicrobial agent; an antiviral agent; an antifungal agent; vitamin; homeopathic agent; anti-inflammatory agent; keratolytic agent; antipruritic agent; pain medicine; steroid; naloxone; and a combination thereof; and/or (b) selected from the group consisting of a penicillin, a cephalosporin, cycloserine, vancomycin, bacitracin, miconazole, ketoconazole, clotrimazole, polymyxin, colistimethate, nystatin, amphotericin B, chloramphenicol, a tetracycline, erythromycin, clindamycin, an aminoglycoside, a rifamycin, a quinolone, trimethoprim, a sulfonamide, zidovudine, gangcyclovir, vidarabine, acyclovir, poly(hexamethylene biguanide), terbinafine, and a combination thereof; (c) an anti-inflammatory agent; and/or (n) an anti-inflammatory agent which is a steroid or a non-steroidal anti-inflammatory drug; and/or (o) an anti-inflammatory agent which is a steroid selected from the group consisting of clobetasol, halobetasol, halcinonide, amcinonide, betamethasone, desoximetasone, diflucortolone, fluocinolone, fluocinonide, mometasone, clobetasone, desonide, hydrocortisone, prednicarbate, triamcinolone, and a pharmaceutically acceptable derivative thereof; and/or (p) an anti-inflammatory agent which is a non-steroidal anti-inflammatory drug selected from the group consisting of aceclofenac, aspirin, celecoxib, clonixin, dexibup6fen, dexketoprofen, diclofenac, diflunisal, droxicam, etodolac, etoricoxib, fenoprofen, flufenamic acid, flurbiprofen, ibuprofen, indomethacin, isoxicam, ketoprofen, ketorolac, licofelone, lornoxicam, loxoprofen, lumiracoxib, meclofenamic acid, mefenamic acid, meloxicam, nabumetone, naproxen, nimesulide, oxaprozin, parecoxib, phenylbutazone, piroxicam, rofecoxib, salsalate, sulindac, tenoxicam, tolfenamic acid, tolmetin, or valdecoxib.

20. An article of manufacture for use in treating coronavirus disease comprising a single dose capsule or tablet containing a single fixed dose of triethylenetetramine disuccinate, wherein the fixed dose is selected from the group consisting of about 350 mg, about 584 mg and about 701 mg of triethylenetetramine disuccinate.

21. The article of manufacture of claim 20, wherein the triethylenetetramine disuccinate is a crystalline form of triethylenetetramine disuccinate.

22. The article of manufacture of claim 20, wherein the triethylenetetramine disuccinate is a triethylenetetramine disuccinate anhydrate.

23. The article of manufacture of claim 20, wherein the capsule or tablet is formulated to provide for a delayed release.

24. The article of manufacture of claim 20, wherein the capsule or tablet is formulated to provide for a sustained release.

25. The article of manufacture of claim 20, wherein the capsule or tablet is formulated in combination with a pharmacokinetic enhancer (PKE) that provides for improved absorption of the triethylenetetramine disuccinate.

26. The article of manufacture of claim 20, further comprising an inhibitor of N-acetylaminotransferase, wherein the inhibitor of N-acetylaminotransferase is an inhibitor of spermine/spermidine N-acetyltransferase (SSAT1) or an inhibitor of spermine/spermidine N-acetyltransferase (SSAT2).

27. The article of manufacture of claim 20, further comprising a promoter of polyamine membrane transport including bergamottin, maringenin, quercetin, other psoralens, piperine, or tetrahydro-piperine that act as enhancers of membrane permeability for increased absorption.

28. An article of manufacture according to claim 20, wherein the fixed dose triethylenetetramine disuccinate capsule or tablet has a shelf-life term of at least about 12 months at room temperature.

29. The article of manufacture according to claim 20, wherein minimum purity of the triethylenetetramine disuccinate over said shelf-life term is least about 98.5% with no degradation product above about 0.5% and no new, unidentified impurities above about 0.1%.

30. The article of manufacture according to claim 29, wherein the shelf-life term is about 12 months.
